(19) United States
(12) Patent Application Publication
(10) Pub. No.: US 2020/0048358 A1
(43) Pub. Date: Feb. 13, 2020

(54) BISPECIFIC ANTIBODY AGAINST OX40 AND CTLA-4

(71) Applicant: ALLIGATOR BIOSCIENCE AB, Lund (SE)

(72) Inventors: Peter Ellmark, Lund (SE); Christina Furebring, Lund (SE); Per Norlen, Lund (SE); Niina Veitonmaki, Lund (SE)

(21) Appl. No.: 16/607,774

(22) PCT Filed: May 1, 2018
(86) PCT No.: PCT/EP2018/061084

§ 371 Date: Oct. 24, 2019

(30) Foreign Application Priority Data
May 2, 2017 (GB) 1706915.4
Oct. 31, 2017 (GB) 1717958.1
Apr. 9, 2018 (GB) 1805873.5

(51) Int. Cl. C07K 16/28; A61K 47/68

(52) U.S. Cl. CPC C07K 16/2878; C07K 16/2818; A61K 47/6803; A61K 47/6849; A61K 2039/505; C07K 2317/92; C07K 2317/33; C07K 2317/75; C07K 2317/732; C07K 2317/31

(57) ABSTRACT

The present invention provides bispecific polypeptides, such as bispecific antibodies, comprising a first binding domain capable of specifically binding to OX40, and a second binding domain capable of specifically binding to CTLA-4. The invention further provides compositions of said bispecific polypeptides, as well as methods and uses of the same.

### FIELD OF INVENTION

The present invention relates to bispecific polypeptides which specifically bind to OX40 and CTLA-4, and in particular to human OX40 and human CTLA-4.

### BACKGROUND

Cancer is a leading cause of premature deaths in the developed world. Immunotherapy of cancer aims to mount an effective immune response against tumour cells. This may be achieved by, for example, breaking tolerance against tumour antigen, augmenting anti-tumour immune responses, and stimulating local cytokine responses at the tumour site. The key effector cell of a long lasting anti-tumour immune response is the activated tumour specific effector T cell. Potent expansion of activated effector T cells can redirect the immune response towards the tumour. In this context, regulatory T cells (Treg) play a role in inhibiting the anti-tumour immunity. Depleting, inhibiting, reverting or inactivating Tregs may therefore provide anti-tumour effects and revert the immune suppression in the tumour microenvironment. Further, incomplete activation of effector T cells by, for example, dendritic cells can cause T cell anergy, which results in an inefficient anti-tumour response, whereas adequate induction by dendritic cells can generate a potent expansion of activated effector T cells, redirecting the immune response towards the tumour. In addition, Natural killer (NK) cells play an important role in tumour immunology by attacking tumour cells with down-regulated human leukocyte antigen (HLA) expression and by inducing antibody dependent cellular cytotoxicity (ADCC). Stimulation of NK cells may thus also reduce tumour growth.

OX40 (otherwise known as CD134 or TNFRSF4) is a member of the TNFR family that is expressed mainly on activated T cells (mostly CD4+ effector T cells, but also CD8+ effector T-cells and regulatory T cells (Tregs)). In mice the expression is constitutive on Tregs, but not in humans. OX40 expression typically occurs within 24 hours of activation (T cell receptor engagement) and peaks after 48-72 hours. OX40 stimulation is important for the survival and proliferation of activated T cells. The only known ligand for OX40 is OX40L, which is mainly expressed on antigen presenting cells, such as dendritic cells and B cells, typically following their activation. The net result of OX40-mediated T cell activation is the induction of a TH1 effector T cell activation profile and a reduction in the activity and/or numbers of Treg cells e.g. via ADCC or ADCP. Overall these effects may contribute to anti-tumour immunity. OX40 is overexpressed on regulatory T cells in many solid tumours, such as melanoma, lung cancer and renal cancer.

OX40 agonist treatment of tumour models in mice has been shown to result in anti-tumour effects and cure of several different cancer forms, including melanoma, glioma, sarcoma, prostate, colon and renal cancers. The data is consistent with a tumour specific T-cell response, involving both CD4+ and CD8+ T cells, similar to the effect seen with CD40 agonist treatments. Addition of IL-12 and other cytokines, and combination with other immunomodulators and chemo/radiotherapy, has been shown to improve the therapeutic effect of OX40 agonist treatment. Evidence from pre-clinical models suggests that the effect of anti-OX40 antibodies is dependent upon activating FcγR. A clinical phase I study testing the mouse anti-human OX40 Clone 9612 in late stage patients that had failed all other therapy has been conducted at the Providence Cancer Centre. The antibody was well-tolerated. Tumour shrinkage and an increase in CD4+ and CD8+ T cell proliferation were observed. The low toxicity may be caused by low half-life and anti-drug antibodies (the antibody was a mouse antibody), but also by the relatively low expression levels of OX40 on non-activated T cells. The anti-tumour effect with this antibody was modest.

Existing antibodies targeting OX40 are in general dependent on cross linking via e.g. Fcgamma Receptors on other cells to induce strong signalling into cells expressing the respective receptor. Thus, they do not signal efficiently when no such cross linking is provided. In addition, prolonged and continuous activation through TNF receptor family members may lead to immune exhaustion.

The T cell receptor CTLA-4, serves as a negative regulator of T cell activation, and is upregulated on the T-cell surface following initial activation. The ligands of the CTLA-4 receptor, which are expressed by antigen presenting cells are the B7 proteins. The corresponding ligand receptor pair that is responsible for the upregulation of T cell activation is CD28-B7. Signalling via CD28 constitutes a costimulatory pathway, and follows upon the activation of T cells, through the T cell receptor recognizing antigenic peptide presented by the MHC complex. By blocking the CTLA-4 interaction to the B7-1 and, or B7-2 ligands, one of the normal check points of the immune response may be removed. The net result is enhanced activity of effector T cells which may contribute to anti-tumour immunity. As with OX40, this may be due to direct activation of the effector T cells but may also be due to a reduction in the activity and/or numbers of Treg cells, e.g. via ADCC or ADCP. Clinical studies have demonstrated that CTLA-4 blockade generates anti-tumour effects, but administration of anti-CTLA-4 antibodies has been associated with toxic side-effects. CTLA-4 is overexpressed on regulatory T cells in many solid tumours, such as melanoma lung cancer and renal cancer.

There is a need for improved therapeutic agents capable of activating the host immune cells in the vicinity of tumour cells, for example as an alternative to existing monospecific drugs that target only one T cell-associated protein (such as OX40 or CTLA-4).

### SUMMARY OF INVENTION

A first aspect of the invention provides a bispecific polypeptide comprising a first binding domain, designated B1, which is capable of specifically binding to OX40, and a second binding domain, designated B2, which is capable of specifically binding to CTLA-4.

Bispecific polypeptides, e.g. antibodies, targeting the two T cell targets CTLA-4 and OX40, have the potential to induce specific activation of the immune system in locations where both targets are over expressed. Notably, CTLA-4 and OX40 are both overexpressed on regulatory T cells (Treg) in the tumour microenvironment, whereas their co-expression on effector T cells is lower. Thus, the bispecific polypeptides of the invention have the potential to selectively target regulatory T cells in the tumour microenvironment.

Targeting Treg cells in the tumour microenvironment with a bispecific polypeptide of the invention also has the potential to deplete or reverse the immune suppressive function of the Tregs. This effect could be mediated by ADCC or ADCP induction via the Fc part of the bispecific antibody of the invention (for example, see Furness et al., 2014 Trends Immunol 35(7):290-8; the disclosures of which are incorporated herein by reference) or by signalling induced via OX40 and/or CTLA-4 and/or by blocking the CTLA-4 signalling pathway (for example, see Walker, 2014, Nature Reviews 11(12):852-63; the disclosures of which are incorporated herein by reference). On effector T cells, on the other hands, the bispecific polypeptides of the invention have the potential to induce activation and increased function both via OX40 stimulation and through CTLA-4 checkpoint blockade.

The net effects of the bispecific polypeptides targeting OX40 and CTLA-4 are thus:
1. A higher degree of immune activation compared to monospecific polypeptides. The immune activation is higher than that induced by the combination of the monospecific polypeptides, i.e. a synergistic activation is achieved.
2. A higher degree of induction of ADCC compared to the monospecific polypeptides in combination.
3. A more directed/localized immune activation. The immune activation only occurs in environments (e.g. tissues) having both high CTLA-4 expression and OX40 expression. The tumour microenvironment is such an environment. This has the potential to increase the immune activation at a tumour site without the toxic side effects associated with activation in other tissues/regions of the body. Thus, the therapeutic window will be increased.

A “polypeptide” is used herein in its broadest sense to refer to a compound of two or more subunit amino acids, amino acid analogues, or other peptidomimetics. The term “polypeptide” thus includes short peptide sequences and also longer polypeptides and proteins. As used herein, the term “amino acid” refers to either natural and/or unnatural or synthetic amino acids, including both D or L optical isomers, and amino acid analogues and peptidomimetics.

The term “bispecific” as used herein means the polypeptide is capable of specifically binding at least two target entities.

In one embodiment, the first and/or second binding domains may be selected from the group consisting of: antibodies or antigen-binding fragments thereof.

For example, the bispecific polypeptide of the invention may comprise:
(i) a first binding domain which comprises or consists of an antibody variable domain or part thereof and a second binding domain which comprises or consists of an antibody variable domain or part thereof; or
(ii) a first binding domain which comprises or consists of an antibody variable domain or part thereof and a second binding domain which is not an antibody variable domain or part thereof.

Thus, in one embodiment the polypeptide is a bispecific antibody.

As used herein, the terms “antibody” or “antibodies” refer to molecules that contain an antigen binding site, e.g. immunoglobulin molecules and immunologically active fragments of immunoglobulin molecules that contain an antigen binding site. Immunoglobulin molecules can be of any type (e.g. IgG, IgE, IgM, IgD, IgA and IgY), class (e.g. IgG1, IgG2, IgG3, IgG4, IgA1 and IgA2) or a subclass of immunoglobulin molecule. Antibodies include, but are not limited to, synthetic antibodies, monoclonal antibodies, single domain antibodies, single chain antibodies, recombinantly produced antibodies, multi-specific antibodies (including bi-specific antibodies), human antibodies, humanized antibodies, chimeric antibodies, intrabodies, scFvs (e.g. including mono-specific and bi-specific, etc.), Fab fragments, F(ab′) fragments, disulfide-linked Fvs (sdFv), anti-idiotypic (anti-Id) antibodies, and epitope-binding fragments of any of the above.

The terms antibody “directed to” or “directed against” are used interchangeably herein and refer to an antibody that is constructed to direct its binding specificity(ies) at a certain target/marker/epitope/antigen, i.e. an antibody that immunospecifically binds to a target/marker/epitope/antigen. Also, the expression antibodies “selective for” a certain target/marker/epitope may be used, having the same definition as “directed to” or “directed against”. A bi-specific antibody directed to (selective for) two different targets/markers/epitopes/antigens binds immunospecifically to both targets/markers/epitopes/antigens. If an antibody is directed to a certain target antigen, such as OX40, it is thus assumed that said antibody could be directed to any suitable epitope present on said target antigen structure.

As used herein, the term “antibody fragment” is a portion of an antibody such as F(ab′) 2, F(ab)₂, Fab′, Fab, Fv, scFv and the like. Regardless of structure, an antibody fragment binds with the same antigen that is recognized by the intact antibody. For example, an anti-OX40 antibody fragment binds to OX40. The term “antibody fragment” also includes isolated fragments consisting of the variable regions, such as the “Fv” fragments consisting of the variable regions of the heavy and light chains and recombinant single chain polypeptide molecules in which light and heavy variable regions are connected by a peptide linker (“scFv proteins”). As used herein, the term “antibody fragment” does not include portions of antibodies without antigen binding activity, such as Fc fragments or single amino acid residues.

ScFv are particularly preferred for inclusion in the bispecific polypeptides of the invention.

Thus, in exemplary embodiments of the bispecific polypeptides of the invention:
(a) binding domain B1 and/or binding domain B2 is an intact IgG antibody (or, together, form an intact IgG antibody);
(b) binding domain B1 and/or binding domain B2 is an Fv fragment (e.g. an scFv);
(c) binding domain B1 and/or binding domain B2 is a Fab fragment; and/or
(d) binding domain B1 and/or binding domain B2 is a single domain antibody (e.g. domain antibodies and nanobodies).

It will be appreciated by persons skilled in the art that the bispecific polypeptides of the invention may be of several different structural formats (for example, see Chan & Carter, 2016, Nature Reviews Immunology 10, 301-316, the disclosures of which are incorporated herein by reference).

In exemplary embodiments, the polypeptide is a bispecific antibody selected from the groups consisting of:
(a) bivalent bispecific antibodies, such as IgG-scFv bispecific antibodies (for example, wherein B1 is an intact IgG and B2 is an scFv attached to B1 at the N-terminus of a light chain and/or at the C-terminus of a light chain and/or at the N-terminus of a heavy chain and/or at the C-terminus of a heavy chain of the IgG, or vice versa);
(b) monovalent bispecific antibodies, such as a DuoBody® (Genmab AS, Copenhagen, Denmark) or ‘knob-in-hole’ bispecific antibody (for example, an scFv-KIH, scFv-KIH^{r}, a BiTE-KIH or a BiTE-KIH^{r} (see Xu et al., 2015, mAbs 7(1):231-242);
(c) scFv₂-Fc bispecific antibodies (such as ADAPTIR™ bispecific antibodies from Aptevo Therapeutics Inc, US);
(d) BiTE/scFv₂ bispecific antibodies;
(e) DVD-Ig bispecific antibodies;
(f) DART-based bispecific antibodies (for example, DART-Fc, DART₂-Fc or DART);
(g) DNL-Fab₃ bispecific antibodies; and
(h) scFv-HSA-scFv bispecific antibodies.

It will be appreciated by persons skilled in the art that the invention also encompasses equivalent formats to those listed above in which one of the binding domains is a non-immunoglobulin binding domain (such as a CD86 polypeptide capable of binding CTLA-4).

For example, the bispecific polypeptide may be an IgG-scFv antibody. The IgG-scFv antibody may be in either VH-VL or VL-VH orientation. In one embodiment, the scFv may be stabilised by a S—S bridge between VH and VL.

Alternatively, the bispecifics polypeptide may be an anti-OX40 IgG (or antigen-binding fragment thereof, such as an scFv) coupled to a CD86 polypeptide.

In one embodiment, binding domain B1 and binding domain B2 are fused directly to each other.

In an alternative embodiment, binding domain B1 and binding domain B2 are joined via a polypeptide linker. For example, a polypeptide linker may be a short linker peptide between about 10 to about 25 amino acids. The linker is usually rich in glycine for flexibility, as well as serine or threonine for solubility, and can either connect the N-terminus of the VH with the C-terminus of the VL, or vice versa. Exemplary linkers include a peptide of amino acid sequence as shown in any one of SEQ ID NOs. 47 to 50, or 144.

The bispecific polypeptides of the invention may be manufactured by any known suitable method used in the art. Methods of preparing bi-specific antibodies of the present invention include BITE (Micromet), DART (MacroGenics), Fcab and Mab² (F-star), Fc-engineered IgGI (Xencor) or DuoBody (based on Fab arm exchange, Genmab). Examples of other platforms useful for preparing bi-specific antibodies include but are not limited to those described in WO 2008/119353 (Genmab), WO 2011/131746 (Genmab) and reported by van der Neut-Kolfschoten et al. (2007, Science 317(5844):1554-7). Traditional methods such as the hybrid hybridoma and chemical conjugation methods (Marvin and Zhu (2005) Acta Pharmacol Sin 26: 649) can also be used. Co-expression in a host cell of two antibodies, consisting of different heavy and light chains, leads to a mixture of possible antibody products in addition to the desired bi-specific antibody, which can then be isolated by, e.g. affinity chromatography or similar methods.

It will be appreciated by persons skilled in the art that the bispecific polypeptide may comprise a human Fc region, or a variant of a said region, where the region is an IgG1, IgG2, IgG3 or IgG4 region, preferably an IgG1 or IgG4 region.

The constant (Fc) regions of the antibodies may mediate the binding of the immunoglobulin to host tissues or factors, including various cells of the immune system (e.g., effector cells) and the first component (Clq) of the classical complement system. The Fc region is preferably a human Fc region, or a variant of a said region. The Fc region may be an IgG1, IgG2, IgG3 or IgG4 region, preferably an IgG1 or IgG4 region. A variant of an Fc region typically binds to Fc receptors, such as FcgammaR and/or neonatal Fc receptor (FcRn) with altered affinity providing for improved function and/or half-life of the polypeptide. The biological function and/or the half-life may be either increased or a decreased relative to the half-life of a polypeptide comprising a native Fc region. Examples of such biological functions which may be modulated by the presence of a variant Fc region include antibody dependent cell cytotoxicity (ADCC), antibody-dependent cellular phagocytosis (ADCP), complement-dependent cytotoxicity (CDC), and/or apoptosis.

An exemplary heavy chain constant region amino acid sequence which may be combined with any VH region sequence disclosed herein (to form a complete heavy chain) is the IgG1 heavy chain constant region sequence reproduced here:

(SEQ ID NO: 135)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFF
LYSKLTVDKSRWQQGNVFSCSVMHEALHNHYTQKSLSLSPGK

Other heavy chain constant region sequences are known in the art and could also be combined with any VH region disclosed herein. For example, a preferred constant region is a modified IgG4 constant region such as that reproduced here:

(SEQ ID NO: 137)
ASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDK
RVESKYGPPCPPCPAPEFLGGPSVFLFPPKPKDTLMISRTPEVTCVVV
DVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYS
RLTVDKSRWQEGNVFSCSVMHEALHNRYTQKSLSLSLGK

This modified IgG4 sequence exhibits reduced FcRn binding and hence results in a reduced serum half-life relative to wild type IgG4. In addition, it exhibits stabilization of the core hinge of IgG4 making the IgG4 more stable, preventing Fab arm exchange.

Another preferred constant region is a modified IgG4 constant region such as that reproduced here:

(SEQ ID NO: 139)
ASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDK
RVESKYGPPCPPCPAPEFLGGPSVFLFPPKPKDTLMISRTPEVTCVVV
DVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYS
RLTVDKSRWQEGNVFSCSVMHEALHNHYTQKSLSLSLGK

This modified IgG4 sequence results in stabilization of the core hinge of IgG4 making the IgG4 more stable, preventing Fab arm exchange

Also preferred is a wild type IgG4 constant region such as that reproduced here:

(SEQ ID NO: 138)
ASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDK
RVESKYGPPCPSCPAPEFLGGPSVFLFPPKPKDTLMISRTPEVTCVVV
DVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYS
RLTVDKSRWQEGNVFSCSVMHEALHNHYTQKSLSLSLGK

An exemplary light chain constant region amino acid sequence which may be combined with any VL region sequence disclosed herein (to form a complete light chain) is the kappa chain constant region sequence reproduced here:

(SEQ ID NO: 136)
RTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSS
PVTKSFNRGEC

Other light chain constant region sequences are known in the art and could also be combined with any VL region disclosed herein.

The antibody, or antigen binding fragment thereof, has certain preferred binding characteristics and functional effects, which are explained in more detail below. Said antibody, or antigen binding fragment thereof, preferably retains these binding characteristics and functional effects when incorporated as part of a bispecific polypeptide of the invention.

In one embodiment, the antigen-binding fragment may be selected from the group consisting of: an Fv fragment (such as a single chain Fv fragment, or a disulphide-bonded Fv fragment), a Fab-like fragment (such as a Fab fragment; a Fab′ fragment or a F(ab)₂ fragment) and domain antibodies.

In one embodiment, the bispecific polypeptide may be an IgG1 antibody with a non-immunoglobulin polypeptide (such as a CTLA-4 binding domain, e.g. CD86 or a mutated form thereof such as SEQ ID NO: 17; see below) fused to the C-terminal part of the kappa chain.

In one embodiment, the bispecific polypeptide may be an IgG1 antibody with a scFv fragment fused to the C-terminal end of the heavy gamma 1 chain.

In one embodiment, the bispecific polypeptide may contain 2-4 scFv binding to two different targets.

It will be appreciated by persons skilled in the art that the T cell targets, CTLA-4 and OX40, may be localised on the surface of a cell. By “localised on the surface of a cell” it is meant that the T cell target is associated with the cell such that one or more region of the T cell target is present on the outer face of the cell surface. For example, the T cell target may be inserted into the cell plasma membrane (i.e. orientated as a transmembrane protein) with one or more regions presented on the extracellular surface. This may occur in the course of expression of the T cell target by the cell. Thus, in one embodiment, “localised on the surface of a cell” may mean “expressed on the surface of a cell.” Alternatively, the T cell target may be outside the cell with covalent and/or ionic interactions localising it to a specific region or regions of the cell surface.

It will be appreciated by persons skilled in the art that the bispecific antibodies of the invention may be capable of inducing antibody dependent cell cytotoxicity (ADCC), antibody-dependent cellular phagocytosis (ADCP), complement-dependent cytotoxicity (CDC), and/or apoptosis.

In a further embodiment, the polypeptide is capable of inducing tumour immunity. This can be tested in vitro in T cell activation assays, e.g. by measuring. IL-2 and IFNγ production. Activation of effector T cells would imply that a tumour specific T cell response can be achieved in vivo. Further, an anti-tumour response in an in vivo model, such as a mouse model would imply that a successful immune response towards the tumour has been achieved.

The antibody may modulate the activity of a cell expressing the T cell target (CTLA-4 or OX40), wherein said modulation is an increase or decrease in the activity of said cell. The cell is typically a T cell. The antibody may increase the activity of a CD4+ or CD8+ effector cell, or may decrease the activity of a regulatory T cell (Treg). In either case, the net effect of the antibody will be an increase in the activity of effector T cells, particularly CD4+ effector T cells. Methods for determining a change in the activity of effector T cells are well known and include, for example, measuring for an increase in the level of T cell IL-2 production or an increase in T cell proliferation in the presence of the antibody relative to the level of T cell IL-2 production and/or T cell proliferation in the presence of a control. Assays for cell proliferation and/or IL-2 production are well known and are exemplified in the Examples.

Standard assays to evaluate the binding ability of ligands towards targets are well known in the art, including for example, ELISAs, Western blots, RIAs, and flow cytometry analysis. The binding kinetics (e.g., binding affinity) of the polypeptide also can be assessed by standard assays known in the art, such as by Surface Plasmon Resonance analysis (SPR).

The terms “binding activity” and “binding affinity” are intended to refer to the tendency of a polypeptide molecule to bind or not to bind to a target. Binding affinity may be quantified by determining the dissociation constant (Kd) for a polypeptide and its target. A lower Kd is indicative of a higher affinity for a target. Similarly, the specificity of binding of a polypeptide to its target may be defined in terms of the comparative dissociation constants (Kd) of the polypeptide for its target as compared to the dissociation constant with respect to the polypeptide and another, non-target molecule.

The value of this dissociation constant can be determined directly by well-known methods, and can be computed even for complex mixtures by methods such as those, for example, set forth in Caceci et al. (Byte 9:340-362, 1984; the disclosures of which are incorporated herein by reference). For example, the Kd may be established using a double-filter nitrocellulose filter binding assay such as that disclosed by Wong & Lohman (Proc. Natl. Acad. Sci. USA 90, 5428-5432, 1993). Other standard assays to evaluate the binding ability of ligands such as antibodies towards targets are known in the art, including for example, ELISAs, Western blots, RIAs, and flow cytometry analysis. The binding kinetics (e.g., binding affinity) of the antibody also can be assessed by standard assays known in the art, such as by Biacore™ system analysis.

A competitive binding assay can be conducted in which the binding of the antibody to the target is compared to the binding of the target by another, known ligand of that target, such as another antibody. The concentration at which 50% inhibition occurs is known as the Ki. Under ideal conditions, the Ki is equivalent to Kd. The Ki value will never be less than the Kd, so measurement of Ki can conveniently be substituted to provide an upper limit for Kd.

Alternative measures of binding affinity include EC50 or IC50. In this context EC50 indicates the concentration at which a polypeptide achieves 50% of its maximum binding to a fixed quantity of target. IC50 indicates the concentration at which a polypeptide inhibits 50% of the maximum binding of a fixed quantity of competitor to a fixed quantity of target. In both cases, a lower level of EC50 or IC50 indicates a higher affinity for a target. The EC50 and IC50 values of a ligand for its target can both be determined by well-known methods, for example ELISA. Suitable assays to assess the EC50 and IC50 of polypeptides are set out in the Examples.

A polypeptide of the invention is preferably capable of binding to its target with an affinity that is at least two-fold, 10-fold, 50-fold, 100-fold or greater than its affinity for binding to another non-target molecule.

The polypeptide of the invention may be produced by any suitable means. For example, all or part of the polypeptide may be expressed as a fusion protein by a cell comprising a nucleotide which encodes said polypeptide.

Alternatively, parts B1 and B2 may be produced separately and then subsequently joined together. Joining may be achieved by any suitable means, for example using the chemical conjugation methods and linkers outlined above. Separate production of parts B1 and B2 may be achieved by any suitable means. For example, by expression from separate nucleotides optionally in separate cells, as is explained in more detail below.

Variants

The bispecific polypeptides or constituent binding domains thereof (such as the OX40 or CTLA-4 binding domains) described herein may comprise a variant or a fragment of any of the specific amino acid sequences recited herein, provided that the polypeptide or binding domain retains binding to its target. In one embodiment, the variant of an antibody or antigen binding fragment may retain the CDR sequences of the sequences recited herein. For example, the anti-OX40 antibody may comprise a variant or a fragment of any of the specific amino acid sequences recited in Tables B, provided that the antibody retains binding to its target. Such a variant or fragment may typically retain the CDR sequences of the said sequence of Table B. The CTLA-4 binding domain may comprise a variant of any of the sequences of Table C, providing that that the binding domain retains binding to its target.

A fragment of any one of the heavy or light chain amino acid sequences recited herein may comprise at least 7, at least 8, at least 9, at least 10, at least 12, at least 15, at least 18, at least 20, at least 25, at least 50, at least 60, at least 70, at least 80, at least 90 or at least 100 consecutive amino acids from the said amino acid sequence.

A variant of any one of the heavy or light chain amino acid sequences recited herein may be a substitution, deletion or addition variant of said sequence. A variant may comprise 1, 2, 3, 4, 5, up to 10, up to 20, up to 30 or more amino acid substitutions and/or deletions from the said sequence. “Deletion” variants may comprise the deletion of individual amino acids, deletion of small groups of amino acids such as 2, 3, 4 or 5 amino acids, or deletion of larger amino acid regions, such as the deletion of specific amino acid domains or other features. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows:

| | | | |
|---|---|---|---|
| Ala, A | aliphatic, hydrophobic, neutral | Met, M | hydrophobic, neutral |
| Cys, C | polar, hydrophobic, neutral | Asn, N | polar, hydrophilic, neutral |
| Asp, D | polar, hydrophilic, charged (−) | Pro, P | hydrophobic, neutral |
| Glu, E | polar, hydrophilic, charged (−) | Gln, Q | polar, hydrophilic, neutral |
| Phe, F | aromatic, hydrophobic, neutral | Arg, R | polar, hydrophilic, charged (+) |
| Gly, G | aliphatic, neutral | Ser, S | polar, hydrophilic, neutral |
| His, H | aromatic, polar, hydrophilic, | Thr, T | polar, hydrophilic, neutral |
| | charged (+) | | |
| Ile, I | aliphatic, hydrophobic, neutral | Val, V | aliphatic, hydrophobic, neutral |
| Lys, K | polar, hydrophilic, charged(+) | Trp, W | aromatic, hydrophobic, neutral |
| Leu, L | aliphatic, hydrophobic, neutral | Tyr, Y | aromatic, polar, hydrophobic |

Amino acids herein may be referred to by full name, three letter code or single letter code.

Preferred “derivatives” or “variants” include those in which instead of the naturally occurring amino acid the amino acid which appears in the sequence is a structural analogue thereof. Amino acids used in the sequences may also be derivatised or modified, e.g. labelled, providing the function of the antibody is not significantly adversely affected.

Derivatives and variants as described above may be prepared during synthesis of the antibody or by post-production modification, or when the antibody is in recombinant form using the known techniques of site-directed mutagenesis, random mutagenesis, or enzymatic cleavage and/or ligation of nucleic acids.

Preferably variants have an amino acid sequence which has more than 60%, or more than 70%, e.g. 75 or 80%, preferably more than 85%, e.g. more than 90 or 95% amino acid identity to a sequence as shown in the sequences disclosed herein. This level of amino acid identity may be seen across the full length of the relevant SEQ ID NO sequence or over a part of the sequence, such as across 20, 30, 50, 75, 100, 150, 200 or more amino acids, depending on the size of the full-length polypeptide.

In connection with amino acid sequences, “sequence identity” refers to sequences which have the stated value when assessed using ClustalW (Thompson et al., 1994, Nucleic Acids Res. 22(22):4673-80; the disclosures of which are incorporated herein by reference) with the following parameters:

Pairwise alignment parameters—Method: accurate, Matrix: PAM, Gap open penalty: 10.00, Gap extension penalty: 0.10;

Multiple alignment parameters—Matrix: PAM, Gap open penalty: 10.00, % identity for delay: 30, Penalize end gaps: on, Gap separation distance: 0, Negative matrix: no, Gap extension penalty: 0.20, Residue-specific gap penalties: on, Hydrophilic gap penalties: on, Hydrophilic residues: GPSNDQEKR. Sequence identity at a particular residue is intended to include identical residues which have simply been derivatised.

Polynucleotides, Vectors and Cells

The invention also relates to polynucleotides that encode all or part of a polypeptide of the invention. Thus, a polynucleotide of the invention may encode any polypeptide as described herein, or all or part of B1 or all or part of B2. The terms “nucleic acid molecule” and “polynucleotide” are used interchangeably herein and refer to a polymeric form of nucleotides of any length, either deoxyribonucleotides or ribonucleotides, or analogues thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or substantially isolated form. By substantially isolated, it is meant that there may be substantial, but not total, isolation of the polypeptide from any surrounding medium. The polynucleotides may be mixed with carriers or diluents which will not interfere with their intended use and still be regarded as substantially isolated.

A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Representative polynucleotides which encode examples of a heavy chain or light chain amino acid sequence of an antibody may comprise or consist of any one of the nucleotide sequences disclosed herein, for example the sequences set out in Table B. Representative polynucleotides which encode the polypeptides shown in Tables D may comprise or consist of the corresponding nucleotide sequences which are also shown in Tables D (intron sequences are shown in lower case). Representative polynucleotides which encode examples of CTLA-4 binding domains may comprise or consist of any one of SEQ ID NOS: 25 to 43 as shown in Table E.

A suitable polynucleotide sequence may alternatively be a variant of one of these specific polynucleotide sequences. For example, a variant may be a substitution, deletion or addition variant of any of the above nucleic acid sequences. A variant polynucleotide may comprise 1, 2, 3, 4, 5, up to 10, up to 20, up to 30, up to 40, up to 50, up to 75 or more nucleic acid substitutions and/or deletions from the sequences given in the sequence listing.

Suitable variants may be at least 70% homologous to a polynucleotide of any one of nucleic acid sequences disclosed herein, preferably at least 80 or 90% and more preferably at least 95%, 97% or 99% homologous thereto. Preferably homology and identity at these levels is present at least with respect to the coding regions of the polynucleotides. Methods of measuring homology are well known in the art and it will be understood by those of skill in the art that in the present context, homology is calculated on the basis of nucleic acid identity. Such homology may exist over a region of at least 15, preferably at least 30, for instance at least 40, 60, 100, 200 or more contiguous nucleotides. Such homology may a exist over the entire length of the unmodified polynucleotide sequence.

Methods of measuring polynucleotide homology or identity are known in the art. For example, the UWGCG Package provides the BESTFIT program which can be used to calculate homology (e.g. used on its default settings) (Devereux et al, 1984, Nucleic Acids Research 12:387-395; the disclosures of which are incorporated herein by reference).

The PILEUP and BLAST algorithms can also be used to calculate homology or line up sequences (typically on their default settings), for example as described in Altschul, 1993, J Mol Evol 36:290-300; Altschul et al, 1990, J Mol Biol 215:403-10, the disclosures of which are incorporated herein by reference).

Software for performing BLAST analysis is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff & Henikoff, 1992, Proc. Natl. Acad. Sci. USA 89:10915-10919; the disclosures of which are incorporated herein by reference) alignments (B) of 50, expectation (E) of 10, M=5, N=4, and a comparison of both strands.

The BLAST algorithm performs a statistical analysis of the similarity between two sequences; see e.g. Karlin & Altschul, 1993, Proc. Natl. Acad. Sci. USA 90:5873-5787; the disclosures of which are incorporated herein by reference. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologue may differ from a sequence in the relevant polynucleotide by less than 3, 5, 10, 15, 20 or more mutations (each of which may be a substitution, deletion or insertion). These mutations may be measured over a region of at least 30, for instance at least 40, 60 or 100 or more contiguous nucleotides of the homologue.

In one embodiment, a variant sequence may vary from the specific sequences given in the sequence listing by virtue of the redundancy in the genetic code. The DNA code has 4 primary nucleic acid residues (A, T, C and G) and uses these to “spell” three letter codons which represent the amino acids the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons. A variant polynucleotide of the invention may therefore encode the same polypeptide sequence as another polynucleotide of the invention, but may have a different nucleic acid sequence due to the use of different codons to encode the same amino acids.

A polypeptide of the invention may thus be produced from or delivered in the form of a polynucleotide which encodes and is capable of expressing it.

Polynucleotides of the invention can be synthesised according to methods well known in the art, as described by way of example in Green & Sambrook (2012, Molecular Cloning—a laboratory manual, 4^{th} edition; Cold Spring Harbor Press; the disclosures of which are incorporated herein by reference).

The nucleic acid molecules of the present invention may be provided in the form of an expression cassette which includes control sequences operably linked to the inserted sequence, thus allowing for expression of the polypeptide of the invention in vivo. These expression cassettes, in turn, are typically provided within vectors (e.g., plasmids or recombinant viral vectors). Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art (see Green & Sambrook, supra).

The invention also includes cells that have been modified to express a polypeptide of the invention. Such cells include transient, or preferably stable higher eukaryotic cell lines, such as mammalian cells or insect cells, lower eukaryotic cells, such as yeast or prokaryotic cells such as bacterial cells. Particular examples of cells which may be modified by insertion of vectors or expression cassettes encoding for a polypeptide of the invention include mammalian HEK293T, CHO, HeLa, NS0 and COS cells. Preferably the cell line selected will be one which is not only stable, but also allows for mature glycosylation and cell surface expression of a polypeptide.

Such cell lines of the invention may be cultured using routine methods to produce a polypeptide of the invention, or may be used therapeutically or prophylactically to deliver antibodies of the invention to a subject. Alternatively, polynucleotides, expression cassettes or vectors of the invention may be administered to a cell from a subject ex vivo and the cell then returned to the body of the subject.

Pharmaceutical Formulations, Therapeutic Uses and Patient Groups

In another aspect, the present invention provides compositions comprising molecules of the invention, such as the antibodies, bispecific polypeptides, polynucleotides, vectors and cells described herein. For example, the invention provides a composition comprising one or more molecules of the invention, such as one or more antibodies and/or bispecific polypeptides of the invention, and at least one pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for parenteral, e.g. intravenous, intramuscular or subcutaneous administration (e.g., by injection or infusion). Depending on the route of administration, the polypeptide may be coated in a material to protect the polypeptide from the action of acids and other natural conditions that may inactivate or denature the polypeptide.

Preferred pharmaceutically acceptable carriers comprise aqueous carriers or diluents. Examples of suitable aqueous carriers that may be employed in the compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. In many cases, it will be preferable to include isotonic agents, for example, sugars, polyalcohols such as mannitol, sorbitol, or sodium chloride in the composition.

A composition of the invention also may include a pharmaceutically acceptable anti-oxidant. These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminium monostearate and gelatin.

Therapeutic compositions typically must be sterile and stable under the conditions of manufacture and storage. The composition can be formulated as a solution, microemulsion, liposome, or other ordered structure suitable to high drug concentration.

Sterile injectable solutions can be prepared by incorporating the active agent (e.g. polypeptide) in the required amount in an appropriate solvent with one or a combination of ingredients enumerated above, as required, followed by sterilization microfiltration. Generally, dispersions are prepared by incorporating the active agent into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active agent plus any additional desired ingredient from a previously sterile-filtered solution thereof.

Particularly preferred compositions are formulated for systemic administration or for local administration. Local administration may be at the site of a tumour or into a tumour draining lymph node. The composition may preferably be formulated for sustained release over a period of time. Thus, the composition may be provided in or as part of a matrix facilitating sustained release. Preferred sustained release matrices may comprise a montanide or γ-polyglutamic acid (PGA) nanoparticles. Localised release of a polypeptide of the invention, optionally over a sustained period of time, may reduce potential autoimmune side-effects associated with administration of a CTLA-4 antagonist.

Compositions of the invention may comprise additional active ingredients as well as a polypeptide of the invention. As mentioned above, compositions of the invention may comprise one or more polypeptides of the invention. They may also comprise additional therapeutic or prophylactic agents.

Also within the scope of the present invention are kits comprising polypeptides or other compositions of the invention and instructions for use. The kit may further contain one or more additional reagents, such as an additional therapeutic or prophylactic agent as discussed above.

The polypeptides in accordance with the present invention maybe used in therapy or prophylaxis. In therapeutic applications, polypeptides or compositions are administered to a subject already suffering from a disorder or condition, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as “therapeutically effective amount”. In prophylactic applications, polypeptides or compositions are administered to a subject not yet exhibiting symptoms of a disorder or condition, in an amount sufficient to prevent or delay the development of symptoms. Such an amount is defined as a “prophylactically effective amount”. The subject may have been identified as being at risk of developing the disease or condition by any suitable means.

In particular, antibodies and bispecific polypeptides of the invention may be useful in the treatment or prevention of cancer. Accordingly, the invention provides an antibody or bispecific polypeptide of the invention for use in the treatment or prevention of cancer. The invention also provides a method of treating or preventing cancer comprising administering to an individual a polypeptide of the invention. The invention also provides an antibody or bispecific polypeptide of the invention for use in the manufacture of a medicament for the treatment or prevention of cancer.

The cancer may be prostate cancer, breast cancer, colorectal cancer, pancreatic cancer, ovarian cancer, lung cancer, cervical cancer, rhabdomyosarcoma, neuroblastoma, multiple myeloma, leukemia, acute lymphoblastic leukemia, melanoma, bladder cancer, gastric cancer, head and neck cancer, liver cancer, skin cancer, lymphoma or glioblastoma.

An antibody or bispecific polypeptide of the present invention, or a composition comprising said antibody or said polypeptide, may be administered via one or more routes of administration using one or more of a variety of methods known in the art. As will be appreciated by the skilled artisan, the route and/or mode of administration will vary depending upon the desired results. Systemic administration or local administration are preferred. Local administration may be at the site of a tumour or into a tumour draining lymph node. Preferred modes of administration for polypeptides or compositions of the invention include intravenous, intramuscular, intradermal, intraperitoneal, subcutaneous, spinal or other parenteral modes of administration, for example by injection or infusion. The phrase “parenteral administration” as used herein means modes of administration other than enteral and topical administration, usually by injection. Alternatively, a polypeptide or composition of the invention can be administered via a non-parenteral mode, such as a topical, epidermal or mucosal mode of administration.

A suitable dosage of an antibody or polypeptide of the invention may be determined by a skilled medical practitioner. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular polypeptide employed, the route of administration, the time of administration, the rate of excretion of the polypeptide, the duration of the treatment, other drugs, compounds and/or materials used in combination with the particular compositions employed, the age, sex, weight, condition, general health and prior medical history of the patient being treated, and like factors well known in the medical arts.

A suitable dose of an antibody or polypeptide of the invention may be, for example, in the range of from about 0.1 μg/kg to about 100 mg/kg body weight of the patient to be treated. For example, a suitable dosage may be from about 1 μg/kg to about 10 mg/kg body weight per day or from about 10 g/kg to about 5 mg/kg body weight per day.

Dosage regimens may be adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Antibodies or polypeptides may be administered in a single dose or in multiple doses. The multiple doses may be administered via the same or different routes and to the same or different locations. Alternatively, antibodies or polypeptides can be administered as a sustained release formulation as described above, in which case less frequent administration is required. Dosage and frequency may vary depending on the half-life of the polypeptide in the patient and the duration of treatment that is desired. The dosage and frequency of administration can also vary depending on whether the treatment is prophylactic or therapeutic. In prophylactic applications, a relatively low dosage may be administered at relatively infrequent intervals over a long period of time. In therapeutic applications, a relatively high dosage may be administered, for example until the patient shows partial or complete amelioration of symptoms of disease.

Combined administration of two or more agents may be achieved in a number of different ways. In one embodiment, the antibody or polypeptide and the other agent may be administered together in a single composition. In another embodiment, the antibody or polypeptide and the other agent may be administered in separate compositions as part of a combined therapy. For example, the modulator may be administered before, after or concurrently with the other agent.

An antibody, polypeptide or composition of the invention may also be used in a method of increasing the activation of a population of cells expressing the first and second T cell target, the method comprising administering to said population of cells a polypeptide or composition of the invention under conditions suitable to permit interaction between said cell and a polypeptide of the invention. The population of cells typically comprises at least some cells which express the first T cell target, typically T cells, and at least some cells which express the second T cell target. The method is typically carried out ex vivo.

For example, an antibody, polypeptide or composition of the invention may also be used in a method of increasing the activation of a population of cells expressing human OX40 and human CTLA-4, the method as described above.

Binding Domains for CTLA-4

The bispecific polypeptides of the invention comprise a binding domain specific for CTLA-4.

CD86 and CD80 may be referred to herein as B7 proteins (B7-2 and B7-1 respectively). These proteins are expressed on the surface of antigen presenting cells and interact with the T cell receptors CD28 and CTLA-4. The binding of the B7 molecules to CD28 promotes T cell activation while binding of B7 molecules to CTLA-4 switches off the activation of the T cell. The interaction between the B7 proteins with CD28 and/or CTLA-4 constitutes a costimulatory signalling pathway which plays an important role in immune activation and regulation. Thus, the B7 molecules are part of a pathway, amenable to manipulation in order to uncouple immune inhibition, thereby enhancing immunity in patients.

The CD86 protein is a monomer and consists of two extracellular immunoglobulin superfamily domains. The receptor binding domain of CD86 has a typical IgV-set structure, whereas the membrane proximal domain has a C1-set like structure. The structures of CD80 and CD86 have been determined on their own or in complex with CTLA-4. The contact residues on the CD80 and CD86 molecules are in the soluble extracellular domain, and mostly located in the beta-sheets and not in the (CDR-like) loops.

SEQ ID NO: 3 is the amino acid sequence of the monomeric soluble extracellular domain of human wild-type CD86. This wild type sequence may optionally lack Alanine and Proline at the N terminus, i.e. positions 24 and 25. These amino acids may be referred to herein as A24 and P25 respectively.

A bispecific polypeptide of the invention has as a polypeptide binding domain a domain which is specific for CTLA-4, a “CTLA-4 binding domain”. Suitable examples of such binding domains are disclosed in WO 2014/207063, the contents of which are incorporated by reference. The binding domain specific for CTLA-4 may also bind to CD28. The term CTLA-4 as used herein typically refers to human CTLA-4 and the term CD28 as used herein typically refers to human CD28. The sequences of human CTLA-4 and human CD28 are set out in SEQ ID NOs: 1 and 2 respectively. The CTLA-4 binding domain of the polypeptide of the present invention may have some binding affinity for CTLA-4 or CD28 from other mammals, for example primate or murine CTLA-4 or CD28.

The CTLA-4 binding domain has the ability to bind to CTLA-4 in its native state and in particular to CTLA-4 localised on the surface of a cell.

“Localised on the surface of a cell” is as defined above.

The CTLA-4 binding domain part of the polypeptide of the invention may comprise or consist of:
(i) the amino acid sequence of SEQ ID NO: 3; or
(ii) an amino acid sequence in which at least one amino acid is changed when compared to the amino acid sequence of SEQ ID NO: 3 provided that said binding domain binds to human CTLA-4 with higher affinity than wild-type human CD86.

In other words, the CTLA-4 binding domain is a polypeptide binding domain specific for human CTLA-4 which comprises or consists of (i) the monomeric soluble extracellular domain of human wild-type CD86, or (ii) a polypeptide variant of said soluble extracellular domain, provided that said polypeptide variant binds to human CTLA-4 with higher affinity than wild-type human CD86.

Accordingly, the CTLA-4 binding domain of the polypeptide of the invention may have the same target binding properties as human wild-type CD86, or may have different target binding properties compared to the target binding properties of human wild-type CD86. For the purposes of comparing such properties, “human wild-type CD86” typically refers to the monomeric soluble extracellular domain of human wild-type CD86 as described in the preceding section.

Human wild-type CD86 specifically binds to two targets, CTLA-4 and CD28. Accordingly, the binding properties of the CTLA-4 binding domain of the polypeptide of the invention may be expressed as an individual measure of the ability of the polypeptide to bind to each of these targets. For example, a polypeptide variant of the monomeric extracellular domain of human wild-type CD86 preferably binds to CTLA-4 with a higher binding affinity than that of wild-type human CD86 for CTLA-4. Such a polypeptide may optionally also bind to CD28 with a lower binding affinity than that of wild-type human CD86 for CD28.

The CTLA-4 binding domain of the polypeptide of the invention is a polypeptide binding domain specific for CTLA-4. This means that it binds to CTLA-4 preferably with a greater binding affinity than that at which it binds to another molecule. The CTLA-4 binding domain preferably binds to CTLA-4 with the same or with a higher affinity than that of wild-type human CD86 for CTLA-4.

Preferably, the Kd of the CTLA-4 binding domain of the polypeptide of the invention for human CTLA-4 will be at least 2-fold, at least 2.5-fold, at least 3-fold, at least 3.5-fold, at least 4-fold, at least 4.5-fold, at least 5-fold, at least 5.5-fold, at least 8-fold or at least 10-fold less than the Kd of wild-type human CD86 for human CTLA-4. Most preferably, the Kd of the CTLA-4 binding domain for human CTLA-4 will be at least 5-fold or at least 10-fold less than the Kd of wild-type human CD86 for human CTLA-4. A preferred method for determining the Kd of a polypeptide for CTLA-4 is SPR analysis, e.g. with a Biacore™ system. Suitable protocols for the SPR analysis of polypeptides are set out in the Examples.

Preferably, the EC50 of the CTLA-4 binding domain of the polypeptide of the invention for human CTLA-4 will be at least 1.5-fold, at least 2-fold, at least 3-fold, at least 5-fold, at least 10-fold, at least 12-fold, at least 14-fold, at least 15-fold, at least 17-fold, at least 20-fold, at least 25-fold or at least 50-fold less than the EC50 of wild-type human CD86 for human CTLA-4 under the same conditions. Most preferably, the EC50 of the CTLA-4 binding domain for human CTLA-4 will be at least 10-fold or at least 25-fold less than the EC50 of wild-type human CD86 for human CTLA-4 under the same conditions. A preferred method for determining the EC50 of a polypeptide for CTLA-4 is via ELISA. Suitable ELISA assays for use in the assessment of the EC50 of polypeptides are set out in the Examples.

Preferably, the IC50 of the CTLA-4 binding domain of the polypeptide of the invention when competing with wild-type human CD86 for binding to human CTLA-4 will be at least 2-fold, at least 3-fold, at least 4-fold, at least 5-fold, at least 10-fold, at least 13-fold, at least 15-fold, at least 50-fold, at least 100-fold, or at least 300-fold less than the IC50 of wild-type human CD86 under the same conditions. Most preferably, the IC50 of the CTLA-4 binding domain will be at least 10-fold or at least 300-fold less than the IC50 of wild-type human CD86 under the same conditions. A preferred method for determining the IC50 of a polypeptide of the invention is via ELISA. Suitable ELISA assays for use in the assessment of the IC50 of polypeptides of the invention are set out in the Examples.

The CTLA-4 binding domain of the polypeptide of the invention may also bind specifically to CD28. That is, the CTLA-4 binding domain may bind to CD28 with greater binding affinity than that at which it binds to another molecule, with the exception of CTLA-4. The CTLA-4 binding domain may bind to human CD28 with a lower affinity than that of wild-type human CD86 for human CD28. Preferably, the Kd of the CTLA-4 binding domain for human CD28 will be at least 2-fold, preferably at least 5-fold, more preferably at least 10-fold higher than the Kd of wild-type human CD86 for human CD28.

The binding properties of the CTLA-4 binding domain of the polypeptide of the invention may also be expressed as a relative measure of the ability of a polypeptide to bind to the two targets, CTLA-4 and CD28. That is, the binding properties of the CTLA-4 binding domain may be expressed as a relative measure of the ability of the polypeptide to bind to CTLA-4 versus its ability to bind to CD28. Preferably the CTLA-4 binding domain has an increased relative ability to bind to CTLA-4 versus CD28, when compared to the corresponding relative ability of human wild-type CD86 to bind to CTLA-4 versus CD28.

When the binding affinity of a polypeptide for both CTLA-4 and CD28 is assessed using the same parameter (e.g. Kd, EC50), then the relative binding ability of the polypeptide for each target may be expressed as a simple ratio of the values of the parameter for each target. This ratio may be referred to as the binding ratio or binding strength ratio of a polypeptide. For many parameters used to assess binding affinity (e.g. Kd, EC50), a lower value indicates a higher affinity. When this is the case, the ratio of binding affinities for CTLA-4 versus CD28 is preferably expressed as a single numerical value calculated according to the following formula:

Binding ratio=[binding affinity for CD28]÷[binding affinity for CTLA-4]

Alternatively, if binding affinity is assessed using a parameter for which a higher value indicates a higher affinity, the inverse of the above formula is preferred. In either context, the CTLA-4 binding domain of the polypeptide of the invention preferably has a higher binding ratio than human wild-type CD86. It will be appreciated that direct comparison of the binding ratio for a given polypeptide to the binding ratio for another polypeptide typically requires that the same parameters be used to assess the binding affinities and calculate the binding ratios for both polypeptides.

Preferably, the binding ratio for a polypeptide is calculated by determining the Kd of the polypeptide for each target and then calculating the ratio in accordance with the formula [Kd for CD28]÷[Kd for CTLA-4]. This ratio may be referred to as the Kd binding ratio of a polypeptide. A preferred method for determining the Kd of a polypeptide for a target is SPR analysis, e.g. with a Biacore™ system. Suitable protocols for the SPR analysis of polypeptides of the invention are set out in the Examples. The binding ratio of the CTLA-4 binding domain of the polypeptide of the invention calculated according to this method is preferably at least 2-fold or at least 4-fold higher than the binding ratio of wild-type human CD86 calculated according to the same method.

Alternatively, the binding ratio for a polypeptide may be calculated by determining the EC50 of the polypeptide for each target and then calculating the ratio in accordance with the formula [EC50 for OX40]÷[EC50 for CTLA-4]. This ratio may be referred to as the EC50 binding ratio of a polypeptide. A preferred method for determining the EC50 of a polypeptide for a target is via ELISA. Suitable ELISA assays for use in the assessment of the EC50 of polypeptides of the invention are set out in the Examples. The binding ratio of the CTLA-4 binding domain of the polypeptide of the invention calculated according to this method is at least 2-fold, at least 3-fold, at least 4-fold, at least 5-fold, at least 6-fold, at least 7-fold, at least 8-fold, at least 9-fold or at least 10-fold higher than the binding ratio of wild-type human CD86 calculated according to the same method.

The CTLA-4 binding domain of the polypeptide of the invention may have the ability to cross-compete with another polypeptide for binding to CTLA-4. For example, the CTLA-4 binding domain may cross-compete with a polypeptide having the amino acid sequence of any one of SEQ ID NOs: 6 to 24 for binding to CTLA-4. Such cross-competing polypeptides may be identified in standard binding assays. For example, SPR analysis (e.g. with a Biacore™ system), ELISA assays or flow cytometry may be used to demonstrate cross-competition.

In addition to the above functional characteristics, the CTLA-4 binding domain of the polypeptide of the invention has certain preferred structural characteristics. The CTLA-4 binding domain either comprises or consists of (i) the monomeric soluble extracellular domain of human wild-type CD86, or (ii) a polypeptide variant of said soluble extracellular domain, provided that said polypeptide variant binds to human CTLA-4 with higher affinity than wild-type human CD86.

A polypeptide variant of the monomeric soluble extracellular domain of human wild-type CD86 comprises or consists of an amino acid sequence which is derived from that of human wild-type CD86, specifically the amino acid sequence of the soluble extracellular domain of human wild-type CD86 (SEQ ID NO: 3), optionally lacking A24 and P25. In particular, a variant comprises an amino acid sequence in which at least one amino acid is changed when compared to the amino acid sequence of SEQ ID NO: 3 (or said sequence lacking A24 and P25). By “changed” it is meant that at least one amino acids is deleted, inserted, or substituted compared to the amino acid sequence of SEQ ID NO: 3 (or said sequence lacking A24 and P25). By “deleted” it is meant that the at least one amino acid present in the amino acid sequence of SEQ ID NO: 3 (or said sequence lacking A24 and P25) is removed, such that the amino acid sequence is shortened by one amino acid. By “inserted” it is meant that the at least one additional amino acid is introduced into the amino acid sequence of SEQ ID NO: 3 (or said sequence lacking A24 and P25), such that the amino acid sequence is lengthened by one amino acid. By “substituted” it is meant that the at least one amino acid in the amino acid sequence of SEQ ID NO: 3 (or said sequence lacking A24 and P25) is replaced with an alternative amino acid.

Typically, at least 1, 2, 3, 4, 5, 6, 7, 8 or 9 amino acids are changed when compared to the amino acid sequence of SEQ ID NO: 3 (or said sequence lacking A24 and P25). Typically, no more than 10, 9, 8, 7, 6, 5, 4, 2 or 1 amino acids are changed when compared to the amino acid sequence of SEQ ID NO: 3 (or said sequence lacking A24 and P25). It will be appreciated that any of these lower limits may be combined with any of these upper limits to define a range for the permitted number of changes compared to the amino acid sequence of SEQ ID NO: 3 (or said sequence lacking A24 and P25). Thus, for example, a polypeptide of the invention may comprise an amino acid sequence in which the permitted number of amino acid changes compared to the amino acid sequence of SEQ ID NO: 3 (or said sequence lacking A24 and P25) is in the range 2 to 3, 2 to 4, 2 to 5, 2 to 6, 2 to 7, 2 to 8, 2 to 9, 2 to 10, 3 to 4, 3 to 5, 3 to 6, and so on.

It is particularly preferred that at least 2 amino acids are changed when compared to the amino acid sequence of SEQ ID NO: 3 (or said sequence lacking A24 and P25). Preferably, the permitted number of amino acid changes compared to the amino acid sequence of SEQ ID NO: 3 (or said sequence lacking A24 and P25) is in the range 2 to 9, 2 to 8 or 2 to 7.

The numbers and ranges set out above may be achieved with any combination of deletions, insertions or substitutions compared to the amino acid sequence of SEQ ID NO: 3 (or said sequence lacking A24 and P25). For example, there may be only deletions, only insertions, or only substitutions compared to the amino acid sequence of SEQ ID NO: 3 (or said sequence lacking A24 and P25), or any mixture of deletions, insertions or substitutions. Preferably the variant comprises an amino acid sequence in which all of the changes compared to the amino acid sequence of SEQ ID NO: 3 (or said sequence lacking A24 and P25) are substitutions. That is, a sequence in which no amino acids are deleted or inserted compared to the sequence of SEQ ID NO: 3 (or said sequence lacking A24 and P25). In the amino acid sequence of a preferred variant, 1, 2, 3, 4, 5, 6, 7, or 8 amino acids are substituted when compared to the amino acid sequence of SEQ ID NO: 3 (or said sequence lacking A24 and P25) and no amino acids are deleted or inserted compared to the sequence of SEQ ID NO: 3 (or said sequence lacking A24 and P25).

Preferably the changes compared to the sequence of SEQ ID NO: 3 (or said sequence lacking A24 and P25) are in the FG loop region (positions 114 to 121) and/or the beta sheet region of SEQ ID NO: 3. The strands of the beta sheet region have the following positions in SEQ ID NO: 3: A:27-31, B:36-37, C:54-58, C′:64-69, C″:72-74, D:86-88, E:95-97, F:107-113, G:122-133.

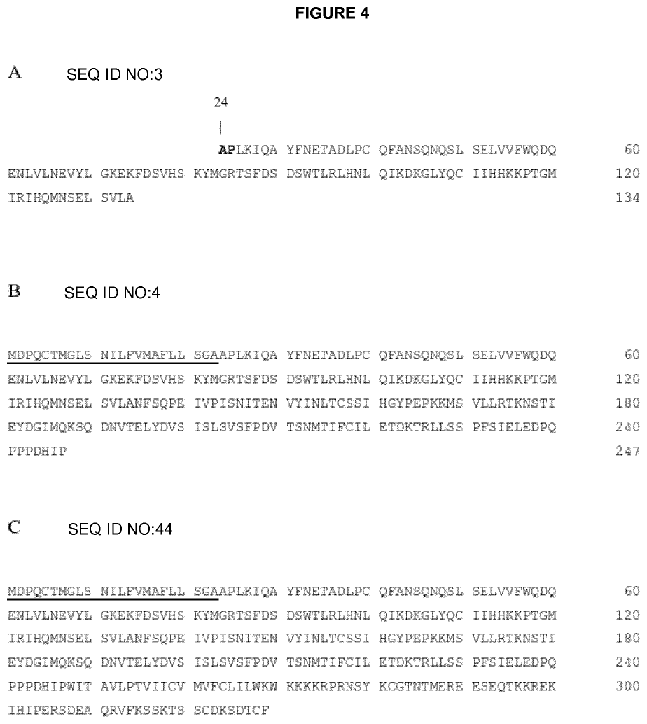

Most preferably, the changes compared to the sequence of SEQ ID NO: 3 (or said sequence lacking A24 and P25) are in one or more of the positions selected from 32, 48, 49, 54, 74, 77, 79, 103, 107, 111, 118, 120, 121, 122, 125, 127 or 134. All numbering of amino acid positions herein is based on counting the amino acids in SEQ ID NO: 4 starting from the N terminus. Thus, the first position at the N terminus of SEQ ID NO: 3 is numbered 24 (see schematic diagram in FIG. 4).

Particularly preferred insertions include a single additional amino acid inserted between positions 116 and 117 and/or a single additional amino acid inserted between positions 118 and 119. The inserted amino acid is preferably Tyrosine (Y), Serine (S), Glycine (G), Leucine (L) or Aspartic Acid (D).

A particularly preferred substitution is at position 122, which is Arginine (R). The polypeptide of the invention preferably includes an amino acid sequence in which at least position 122 is substituted compared to the amino acid sequence of SEQ ID NO: 3 (or said sequence lacking A24 and P25). The most preferred substitution at position 122 is to replace Arginine (R) with Lysine (K) or Asparagine (N), ranked in order of preference. This substitution may be referred to as R122K/N.

Other preferred substitutions are at positions 107, 121, and 125, which are Leucine (L), Isoleucine (I) and Glutamic acid (Q), respectively. In addition to the substitution at position 122, the polypeptide of the invention preferably includes an amino acid sequence in which at least one of the amino acids at positions 107, 121 and 125 is also substituted compared to the amino acid sequence of SEQ ID NO: 3 (or said sequence lacking A24 and P25). The amino acid sequence of the polypeptide of the invention may also be substituted at one or more of positions 32, 48, 49, 54, 64, 74, 77, 79, 103, 111, 118, 120, 127 and 134.

The most preferred substitution at position 107 is to replace Leucine (L) with Isoleucine (I), Phenylalanine (F) or Arginine (R), ranked in order of preference. This substitution may be referred to as L107I/F/R. Similar notation is used for other substitutions described herein. The most preferred substitution at position 121 is to replace Isoleucine (I) with Valine (V). This substitution may be referred to as I121V.

The most preferred substitution at position 125 is to replace Glutamine (Q) with Glutamic acid (E). This substitution may be referred to as Q125E.

Other substitutions which may be preferred in the amino acid sequence of the polypeptide of the invention include: F32I, Q48L, S49T, V54I, V64I, K74I/R, S77A, H79D/S/A, K103E, I111V, T118S, M120L, N127S/D and A134T.

Particularly preferred variants of said soluble extracellular domain of human wild-type CD86 comprise or consist of any one of the amino acid sequences of SEQ ID NOs: 6 to 24, as shown in Table C.

The amino acid sequences shown in SEQ ID NOs: 6 to 14 may optionally include the additional residues AP at the N-terminus. The amino acid sequences shown in SEQ ID NOs: 15 to 24 may optionally lack the residues AP at the N-terminus. In either case, these residues correspond to A24 and P25 of SEQ ID NO: 3.

The CTLA-4 binding domain of the polypeptide of the invention may comprise or consist of any of the above-described variants of said soluble extracellular domain of human wild-type CD86. That is, the CTLA-4 binding domain of the polypeptide of the invention may comprise or consist of the amino acid sequence of any one of SEQ ID NOs: 6 to 24, as shown in Table C.

The binding domain may modulate signalling from CTLA-4, for example when administered to a cell expressing CTLA-4, such as a T cell. Preferably the binding domain reduces, i.e. inhibits or blocks, said signalling and thereby increases the activation of said cell. Changes in CTLA-4 signalling and cell activation as a result of administration of a test agent (such as the binding domain) may be determined by any suitable method. Suitable methods include assaying for the ability of membrane-bound CD86 (e.g. on Raji cells) to bind and signal through CTLA-4 expressed on the surface of T cells, when in the presence of a test agent or in the presence of a suitable control. An increased level of T cell IL-2 production or an increase in T cell proliferation in the presence of the test agent relative to the level of T cell IL-2 production and/or T cell proliferation in the presence of the control is indicative of reduced signalling through CTLA-4 and increased cell activation. A typical assay of this type is disclosed in Example 9 of US20080233122.

Binding Domains for OX40

The bispecific binding molecules of the invention may incorporate as a binding domain (for example, as B1) any OX40 binding domain, for example an anti-OX40 antibody.

The antibody, or antigen binding fragment thereof, that binds specifically to OX40 has certain preferred binding characteristics and functional effects, which are explained in more detail below. Said antibody, or antigen binding fragment thereof, preferably retains these binding characteristics and functional effects when incorporated as part of a bispecific antibody of the invention. This binding domain may also be provided independently of the bispecific molecules of the invention.

The antibody preferably specifically binds to OX40, i.e. it binds to OX40 but does not bind, or binds at a lower affinity, to other molecules. The term OX40 as used herein typically refers to human OX40. The sequence of human OX40 is set out in SEQ ID NO:51 (corresponding to GenBank: NP_003318.1). The antibody may have some binding affinity for OX40 from other mammals, such as OX40 from a non-human primate (for example Macaca fascicularis (cynomolgus monkey), Macaca mulatta). The antibody preferably does not bind to murine OX40 and/or does not bind to other human TNFR superfamily members, for example human CD137 or CD40.

The antibody has the ability to bind to OX40 in its native state and in particular to OX40 localised on the surface of a cell. Preferably, the antibody will bind specifically to OX40. That is, an antibody of the invention will preferably bind to OX40 with greater binding affinity than that at which it binds to another molecule.

“Localised on the surface of a cell” is as defined above.

The antibody may modulate the activity of a cell expressing OX40, wherein said modulation is an increase or decrease in the activity of said cell, as defined above. The cell is typically a T cell. The antibody may increase the activity of a CD4+ or CD8+ effector cell, or may decrease the activity of a regulatory T cell (T reg), as described above.

In either case, the net effect of the antibody will be an increase in the activity of effector T cells, particularly CD4+ effector T cells. Methods for determining a change in the activity of effector T cells are well known and are described above.

The antibody preferably binds to human OX40 with a Kd value which is less than 50×10⁻¹⁰M or less than 25×10⁻¹⁰M, more preferably less than 10, 9, 8, 7, or 6×10⁻¹⁰M, most preferably less than 5×10⁻¹⁰M.

For example, the antibody preferably does not bind to murine OX40 or any other TNFR superfamily member, such as CD137 or CD40. Therefore, typically, the Kd for the antibody with respect to human OX40 will be 2-fold, preferably 5-fold, more preferably 10-fold less than Kd with respect to the other, non-target molecule, such as murine OX40, other TNFR superfamily members, or any other unrelated material or accompanying material in the environment. More preferably, the Kd will be 50-fold less, even more preferably 100-fold less, and yet more preferably 200-fold less.

The value of this dissociation constant can be determined directly by well-known methods, as described above.

The polypeptides of the invention is preferably capable of binding to its target with an affinity that is at least two-fold, 10-fold, 50-fold, 100-fold or greater than its affinity for binding to another non-target molecule.

In summary therefore, the antibody preferably exhibits at least one of the following functional characteristics:
I. binding to human OX40 with a K_{D} value which is less than 10×10⁻¹⁰M;
II. does not bind to murine OX40;
III. does not bind to other human TNFR superfamily members, for example human CD137 or CD40.

The antibody is specific for OX40, typically human OX40 and may comprise any one, two, three, four, five or all six of the following:
(a) a heavy chain CDR1 sequence which is 8 amino acids in length and comprises the consensus sequence: “G, F, T, F, G/Y/S, G/Y/S, Y/S, Y/S/A”;
(b) a heavy chain CDR2 sequence which is 8 amino acids in length and comprises the consensus sequence: “I, G/Y/S/T, G/S/Y, S/Y, G/S/Y, G/S/Y, G/S/Y, T”;
(c) a heavy chain CDR3 sequence which is 9 to 17 amino acids in length and which comprises the consensus sequence of: “A, R, G/Y/S/H, G/Y/F/V/D, G/Y/P/F, −/H/S, −/N/D/H, −/Y/G, −/Y, −/Y, −/W/AN, −/A/Y, −/D/A/Y/G/H/N, Y/S/W/A/T, L/M/I/F, D, Y”. Preferred heavy chain CDR3 sequences within this definition include a CDR3 sequence of 10 amino acids in length which comprises the consensus sequence “A, R, Y/H, D, Y, A/Y/G, S/W/A, M/L, D, Y” or a CDR3 sequence of 11 amino acids in length which comprises the consensus sequence “A, R, G/Y, V/F/Y, P, H, G/Y/H, Y, F/I, D, Y”;
(d) a light chain CDR1 sequence which consists of the sequence: “Q, S, I, S, S, Y”;
(e) a light chain CDR2 sequence which consists of the sequence: “A, A, S”;
(f) a light chain CDR3 sequence which is 8 to 10 amino acids in length and comprises the consensus sequence: “Q,Q, S/Y/G, −/Y/H/G, −/S/Y/G/D/W, S/Y/G/D, S/Y/G/T, P/L, Y/S/H/L/F, T”. A preferred example a light chain CDR3 sequence within this definition consists of the sequence “Q, Q, S, Y, S, T, P, Y, T”

The antibody may comprise at least a heavy chain CDR3 as defined in (c) and/or a light chain CDR3 as defined in (f). The antibody may comprise all three heavy chain CDR sequences of (a), (b) and (c) and/or all three light chain CDR sequences of (d), (e) and (f).

Exemplary CDR sequences are recited in tables A(1) and A(2), SEQ ID NOs: 52 to 88.

Preferred anti-OX40 antibodies may comprise at least a heavy chain CDR3 as defined in any individual row of Table A(1) and/or a light chain CDR3 as defined in in any individual row of Table A(2). The antibody may comprise all three heavy chain CDR sequences shown in an individual row of Table A(1) (that is, all three heavy chain CDRs of a given “VH number”) and/or all three light chain CDR sequences shown in an individual row of Table A(2) (that is, all three light chain CDRs of a given “VL number”).

Examples of complete heavy and light chain variable region amino acid sequences are shown in Table B. Exemplary nucleic acid sequences encoding each amino acid sequence are also shown. The numbering of said VH and VL regions in Table B corresponds to the numbering system used as in Table A(1) and (2). Thus, for example, the amino acid sequence for “1167, light chain VL” is an example of a complete VL region sequence comprising all three CDRs of VL number 1167 shown in Table A(2) and the amino acid sequence for “1166, heavy chain VH” is an example of a complete VH region sequence comprising all three CDRs of VH number 1166 shown in Table A(1).

Preferred anti-OX40 antibodies of the invention include a VH region which comprises all three CDRs of a particular VH number and a VL region which comprises all three CDRs of a particular VL number. For example:
an antibody may comprise all three CDRs of VH number 1166 and all three CDRs of VL number 1167. Such an antibody may be referred to as 1166/1167. Such an antibody may preferably comprise the corresponding complete VH and VL sequences of 1166 and 1167 as shown in Table B (SEQ ID NOs: 91 and 89).
an antibody may comprise all three CDRs of VH number 1170 and all three CDRs of VL number 1171. Such an antibody may be referred to as 1170/1171. Such an antibody may preferably comprise the corresponding complete VH and VL sequences of 1170 and 1171 as shown in Table B (SEQ ID NOs: 95 and 93).
an antibody may comprise all three CDRs of VH number 1164 and all three CDRs of VL number 1135. Such an antibody may be referred to as 1164/1135. Such an antibody may preferably comprise the corresponding complete VH and VL sequences of 1164 and 1135 as shown in Table B (SEQ ID NOs: 99 and 97)
an antibody may comprise all three CDRs of VH number 1168 and all three CDRs of VL number 1135. Such an antibody may be referred to as 1168/1135. Such an antibody may preferably comprise the corresponding complete VH and VL sequences of 1168 and 1135 as shown in Table B (SEQ ID NOs: 101 and 97)
an antibody may comprise all three CDRs of VH number 1482 and all three CDRs of VL number 1483. Such an antibody may be referred to as 1482/1483. Such an antibody may preferably comprise the corresponding complete VH and VL sequences of 1482 and 1483 as shown in Table B (SEQ ID NOs: 105 and 103).
an antibody may comprise all three CDRs of VH number 1490 and all three CDRs of VL number 1135. Such an antibody may be referred to as 1490/1135. Such an antibody may preferably comprise the corresponding complete VH and VL sequences of 1490 and 1135 as shown in Table B (SEQ ID NOs: 107 and 97).
an antibody may comprise all three CDRs of VH number 1514 and all three CDRs of VL number 1515. Such an antibody may be referred to as 1514/1515. Such an antibody may preferably comprise the corresponding complete VH and VL sequences of 1514 and 1515 as shown in Table B (SEQ ID NOs: 111 and 109).
an antibody may comprise all three CDRs of VH number 1520 and all three CDRs of VL number 1135. Such an antibody may be referred to as 1520/1135. Such an antibody may preferably comprise the corresponding complete VH and VL sequences of 1520 and 1135 as shown in Table B (SEQ ID NOs: 113 and 97).
an antibody may comprise all three CDRs of VH number 1524 and all three CDRs of VL number 1525. Such an antibody may be referred to as 1524/1525. Such an antibody may preferably comprise the corresponding complete VH and VL sequences of 1524 and 1525 as shown in Table B (SEQ ID NOs: 117 and 115).
an antibody may comprise all three CDRs of VH number 1526 and all three CDRs of VL number 1527. Such an antibody may be referred to as 1526/1527. Such an antibody may preferably comprise the corresponding complete VH and VL sequences of 1526 and 1527 as shown in Table B (SEQ ID NOs: 121 and 119).
an antibody may comprise all three CDRs of VH number 1542 and all three CDRs of VL number 1135. Such an antibody may be referred to as 1542/1135. Such an antibody may preferably comprise the corresponding complete VH and VL sequences of 1542 and 1135 as shown in Table B (SEQ ID NOs: 123 and 97).

The antibody may comprise a variant or a fragment of any of the specific amino acid sequences recited in Table B, provided that the antibody binds to human OX40 and exhibits at least one of functional characteristics I to III. Such a variant or fragment may typically retain the CDR sequences of the said sequence of Table B.

A fragment of any one of the heavy or light chain amino acid sequences shown in Table B may comprise at least 7, at least 8, at least 9, at least 10, at least 12, at least 15, at least 18, at least 20, least 25, at least 50, at least 60, at least 70, at least 80, at least 90 or at least 100 consecutive amino acids from the said amino acid sequence.

A variant of any one of the heavy or light chain amino acid sequences shown in Table B may be a substitution, deletion or addition variant of said sequence, as defined above.

The antibody may bind to the same epitope as any of the specific antibodies described herein. Preferably it binds to the same epitope as any one of the antibodies designated 1166/1167, 1170/1171, 1164/1135, 1168/1135, 1482/1483, 1490/1135, 1514/1515, 1520/1135, 1524/1525, 1526/1527 and 1542/1135.

Exemplary heavy chain constant region amino acid sequences which may be combined with any VH region sequence disclosed herein (to form a complete heavy chain), such as the IgG1 heavy chain constant region sequence, are described above.

Exemplary light chain constant region amino acid sequences which may be combined with any VL region sequence disclosed herein (to form a complete light chain), such as the kappa chain constant region sequence, are described above.

The Bispecific Polypeptides of the Invention

The polypeptides of the invention comprise binding domains which are specific for OX40 and CTLA-4, i.e. B1 is specific for OX40 and B2 is specific for CTLA-4.

For example, the bispecific polypeptide may be Antibody “1166/1261” comprising an IgG light chain fused to a CD86 domain, of SEQ ID No:125, and an IgG heavy chain comprising a VH region of of SEQ ID No:91. Alternatively, the bispecific polypeptide may comprise variants of said sequences, for example having at least 70% sequence identity with the SEQ ID No:125 and/or 91, e.g. at least 80%, at least 85%, at least 90%, at least 95%, at least 97%, at least 98%, or at least 99% sequence identity with the SEQ ID No:125 and/or 91 sequence identity with the SEQ ID No:125 and/or 9.

The bispecific polypeptide of the embodiment is capable of modulating the activity of cells of the immune system to a greater extent than an individual agonist of OX40 or CTLA-4 alone, or than a combination of such individual agonists. In particular, administration of the bispecific polypeptide produces a higher level of effector T cell activity, particular CD4+ effector T cell activity. The increase in effector T cell activity is also more localised than that which results from administration of an individual OX40 or CTLA-4 agonist alone (or a combination thereof), because the bispecific polypeptide exerts the greatest effect only in a microenvironment in which CTLA-4 and OX40 are both highly expressed. Tumours are such a microenvironment. Tumour infiltrating regulatory T cells (Tregs) express high levels of CTLA-4 and OX40, and higher than effector T cells (both CD4 and CD8).

The increase in effector T cell activity may result directly from stimulation of the effector T cells via activation of the OX40 pathway or via blockade of the CTLA-4 inhibition pathway, or may result indirectly from depletion or down-regulation of Tregs, thereby reducing their immunosuppressive effect. Depletion/down-regulation of Tregs may be mediated by antibody dependent cellular phagocytosis (ADCP) or antibody dependent cellular cytotoxicity (ADCC) mechanisms. The high expression of both CTLA-4 and OX40 on Tregs, compared to effector T cells, may induce a significantly higher killing of Tregs compared to the monospecific antibodies. Effector T cells, having lower expression of CTLA-4 and OX40 will not be depleted by this mechanism. Overall, the result will be a very powerful, localised immune activation for the immediate generation of tumouricidal activity.

Measurement of the effect of a bispecific polypeptide of the invention on cells of the immune system may be achieved with any suitable assay. For example, increased activity of effector T cells may be measured by assays as described above in respect of individual components B1 and B2 of the bispecific polypeptide, and include measurement of proliferation or IL-2 production by CD4+ and/or CD8+ T cells in the presence of the bispecific polypeptide relative to a control. An increase of proliferation or IL-2 production relative to control is indicative of increased cell activation. A typical assay of this type is disclosed in Example 9 of US20080233122. Assays for cell proliferation and/or IL-2 production are well known and are also exemplified in the Examples. When assessed in the same assay, the bispecific molecule will typically induce an increase in the activity of an effector T cell which is at least 1.5 fold higher or at least 2 fold higher, more preferably 3 fold higher, most preferably 5 fold higher than the increase in activity of an effector T cell induced by a combination of monospecific agents binding to the same targets.

The bispecific polypeptide of the invention is capable of specifically binding to both human CTLA-4 and human OX40, and comprises B1 and B2 as defined above.

By “capable of specifically binding to both CTLA-4 and OX40”, it is meant that part B1 specifically binds to OX40 and part B2 specifically binds to CTLA-4, in accordance with the definitions provided for each part above. Preferably the binding characteristics of parts B1 and B2 for their respective targets are unchanged or substantially unchanged when they are present as part of a polypeptide of the invention, when compared to said characteristics for parts B1 and B2 when present as separate entities.

Typically, this means that the bispecific molecule will have a Kd for OX40 which is preferably substantially the same as the Kd value for OX40 of B1 when present alone. Alternatively, if the bispecific molecule has a Kd for OX40 which is increased relative to the Kd for OX40 of B1 when present alone, then the increase is by no more than 10 fold, preferably no more than 9 fold, 8 fold, 7 fold, 6 fold, 5 fold, 4 fold, 3 fold or 2 fold. The bispecific molecule preferably binds to human OX40 with a Kd value which is less than 50×10⁻¹⁰M, more preferably less than 25×10⁻¹⁰M, most preferably less than 20×10⁻¹⁰M. In addition, the bispecific molecule will independently have a Kd for CTLA-4 which is preferably substantially the same as the Kd value for CTLA4 of B2 when present alone. Alternatively, if the bispecific molecule has a Kd for CTLA-4 which is increased relative to the Kd for CTLA-4 of B2 when present alone, then the increase is by no more than 3 fold, preferably no more than 2 fold. The bispecific molecule preferably binds to human CTLA-4 with a Kd value which is less than 60×10⁻⁹M, more preferably less than 25×10⁻⁹M, most preferably less than 10×10⁻⁹M.

In other words, the bispecific molecule may have a Kd for OX40 which is less than 50×10⁻¹⁰M, 25×10⁻¹⁰M, or 20×10⁻¹⁰M and independent have a Kd for CTLA-4 which is less than 60×10⁻⁹M, 25×10⁻⁹M, or 10×10⁻⁹M. It will be appreciated that any of the Kd values recited for OX40 may be independently combined with any of the Kd values recited for CTLA-4 to describe the binding characteristics of a given bispecific molecule. Similarly, any of the recited fold changes in OX40 binding may be independently combined with any of the recited fold changes in CTLA-4 binding to describe the binding characteristics of a given bispecific molecule.

The binding characteristics of parts B1 and B2 when present as part a polypeptide of the invention may be assessed by any suitable assay. In particular, the assays set out above for each separate part may also be applied to B1 and B2 when they are present as part of a polypeptide of the invention. Suitable assays for assessing the binding characteristics of bispecific polypeptides of the invention are also set out in the Examples.

The bispecific molecule potently activates the immune system when in a microenvironment in which both OX40 and CTLA-4 are highly expressed. Typically, the bispecific molecule will increase the activity of a CD4+ or CD8+ effector cell, or may decrease the activity of a regulatory T cell (T reg). In either case, the net effect of the antibody will be an increase in the activity of effector T cells, particularly CD4+ effector T cells. When assessed in the same assay, the bispecific molecule will typically induce an increase in the activity of an effector T cell which is at least 1.5 fold higher or at least 1.7 fold higher, more preferably 4.5 fold higher, most preferably 7 fold higher than the increase in activity of an effector T cell induced by a combination of monospecific agents binding to the same targets.

Methods for determining a change in the activity of effector T cells are well known and are as described earlier. Assays for cell proliferation and/or IL-2 production are well known and are exemplified in the Examples.

For example, the polypeptide may be capable of specifically binding to both CTLA-4 and OX40, and B1 may be an antibody, or antigen binding fragment thereof, specific for OX40; and B2 may be a polypeptide binding domain specific for CTLA-4, which comprises or consists of:
i) the amino acid sequence of SEQ ID NO: 3; or
ii) an amino acid sequence in which at least one amino acid is changed when compared to the amino acid sequence of SEQ ID NO: 3 provided that said binding domain binds to human CTLA-4 with higher affinity than wild-type human CD86.

The CTLA-4 specifically bound by the polypeptide may be primate or murine, preferably human, CTLA-4, and/or the OX40 specifically bound by the polypeptide may be primate, preferably human, OX40.

The bispecific polypeptide of the invention may comprise the OX40 binding domain and the CTLA-4 binding domain arranged together in any suitable format. It will be appreciated that in any given bispecific format, the OX40 binding domain and the CTLA-4 binding domain may each independently be a whole antibody or an antigen binding portion thereof. Irrespective of the particular bispecific format used, bispecific polypeptides and antibodies described herein may typically be referred to by a numbering scheme based on the composition of the OX40 binding domain (which may be referred to as binding domain 1) and the composition of the CTLA-4 binding domain (which may be referred to as binding domain 2). The numbering scheme is therefore typically in the form VH1/VL1 for the OX40 binding domain (binding domain 1) and VH2/VL2 for the CTLA-4 binding domain (binding domain 2), written together as VH1/VL1-VH2/VL2. It will be appreciated that this numbering scheme does not reflect the total number of binding domains present in the bispecific polypeptide or antibody nor the presence or absence of any constant regions in the bispecific polypeptide or antibody, both of which are determined by the particular format of bispecific antibody that is used. The total number of binding domains and the presence or absence of constant regions may be in accordance with any suitable bispecific antibody format known in the art.

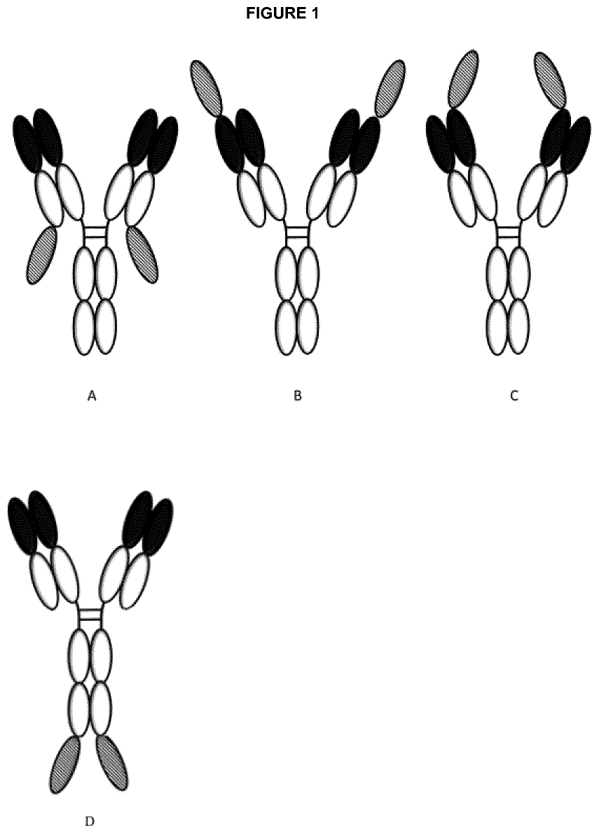

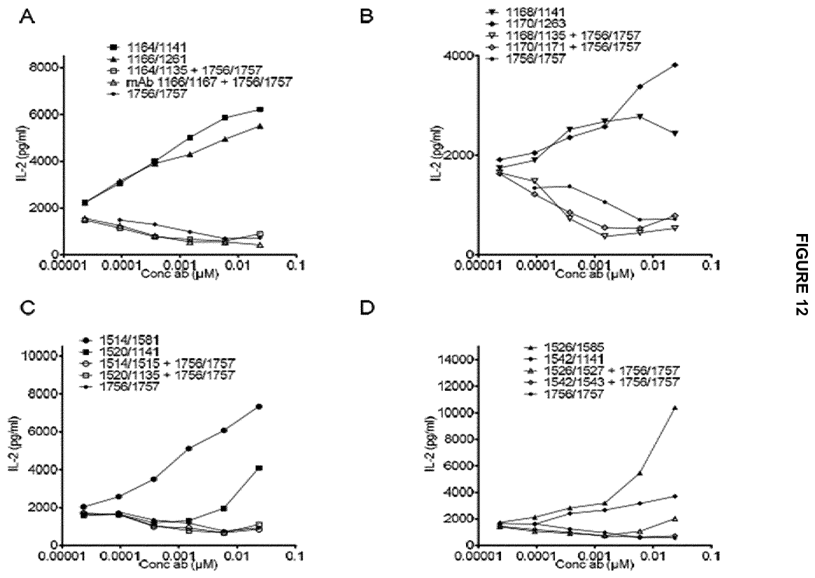

Many suitable formats of bispecific polypeptides or antibodies are known in the art and the bispecific polypeptide of the invention may be in any of these formats. Suitable formats include those described in FIGS. 1 and 14 (see also Kontermann & Brinkmann, 2015, Drug Discov Today. 838-847; the disclosures of which are incorporated herein by reference).

In FIG. 14, constant regions are shown as filled light grey; variable heavy chain region VH1 is shown as checkered black and white; variable light chain region VL1 is shown as filled white; variable heavy chain region VH2 is shown as filled black; and variable light chain region VL2 is shown as white with diagonal lines. Thus, OX40 binding domains (referred to as binding domain 1) are typically represented as a pair of a checkered black and white domain with a filled white domain; CTLA-4 binding domains (referred to as binding domain 2) are typically represented as a pair of a filled black domain and a white domain with diagonal lines. However, in all of the formats shown, it will be appreciated that binding domains 1 and 2 may be switched. That is, an OX40 binding domain may occur in any position shown in FIG. 14 for a CTLA-4 domain, and vice versa.

A preferred format for the bispecific polypeptide is a kih or “knob-in-hole” arrangement, which is the first shown in the second row of FIG. 14. In this arrangement, the CH3 domain of the heavy chain of each antibody is mutated to allow heterodimerisation between a heavy chain from the anti-OX40 antibody and a heavy chain from the anti-CTLA-4 antibody. Each heavy chain associates with its corresponding light chain to form one complete OX40 binding domain and one complete CTLA-4 binding domain. Modifications may be made to the heavy chain CH1 regions to promote association with the correct light chain. Kih format bispecific antibodies are well-known in the art. See for example Ridgway et al 1996; Protein Eng 9:617-621, the disclosures of which are incorporated herein by reference.

Another preferred format for the bispecific antibody of the invention is scFv₂-Fc format, which is the second shown in the second row of FIG. 14. In this arrangement, one scFv specific for each target is fused to constant immunoglobulin domains. The single chains may be fused to the Fc region of the heavy chain, with one specificity fused to the N-terminal end and the other specificity fused to the C-terminal end of the Fc region (Park et al., 2000, Mol Immunol 37(18):1123-30; the disclosures of which are incorporated herein by reference).

Another preferred format for the bispecific polypeptide of the invention is the BITE/scFv₂ format which is the third format shown in the second row of FIG. 14. In this arrangement two scFv, one specific for OX40 and the other specific for CTLA-4, are fused together with a linker (Brischwein et al., 2007, J Immunother 30(8):798-807; the disclosures of which are incorporated herein by reference). The linker may optionally include a protein that increases solubility and serum half-life, such as human serum albumin (HSA), creating scFv-HSA-scFv bispecific antibodies, as shown in the fourth row of FIG. 14.

Another preferred format for the bispecific polypeptide of the invention is double variable domain (DVD) immunoglobulins, which is the fourth format shown in the second row of FIG. 14. In this arrangement, the second variable domain (VL2) is fused to the first variable light chain (VL1), and the second variable heavy chain (VH2) is fused to the first variable heavy chain (VH1) of an IgG molecule. VH1 and VL1 form binding site 1 and VH2 and VL2 form binding site 2, thus creating a bispecific antibody (Wu, 2007, Nat Biotechnol 25(11):1290-7; the disclosures of which are incorporated herein by reference).

Another preferred format for the bispecific polypeptide of the invention is the Dual affinity retargeting (DART) format in which the VH1 is fused to VL2 and VH2 fused to VL1 with a short peptide linker forcing them to form VH1/VL1 and VH2/VL2 binding sites. This construct may be stabilized by formation of a disulphide bridge between the binding sites. The DART format may be fused to IgG Fc domains, creating monovalent bispecific antibodies (DART-Fc) or bivalent bispecific antibodies (DART₂-Fc) (Moore et al., 2011, Blood 117(17):4542-51). The DART, DART-Fc and DART₂-Fc formats are shown in the third row of FIG. 14.

Another preferred format for the bispecific polypeptide of the invention is bispecific antibodies generated by the dock and lock technology (DNL). cAMP dependent protein kinase A and A kinase anchoring protein can be fused to antibodies, Fab fragments or scFv for each target, thereby generating multivalent bispecific antibodies, e.g. DNL-Fab₃ (Chang et al., 2007, Clin Cancer Res 13(18 Pt 2):5586s-5591s, as shown in the fourth row of FIG. 14; the disclosures of which are incorporated herein by reference).

A particularly preferred format for the bispecific polypeptide of the invention is the scFv-IgG format. Four different possible arrangements of this format are shown in the top row of FIG. 14. As shown in FIG. 14, in scFv-IgG format the anti-OX40 antibody is a whole IgG molecule and the anti-CTLA-4 antibody is an scFv antibody connected to the anti-OX40 antibody at any one of four general locations (heavy chain constant region; light chain constant region; heavy chain variable region; light chain variable region). In each case, the reverse arrangement is also envisaged. That is, with the anti-CTLA-4 antibody as a whole IgG and the anti-OX40 antibody as an scFv connected to the anti-CTLA-4 antibody at any one of the four general locations. In the scFv-IgG format, the whole IgG molecule may be joined directly to the scFv or may be joined indirectly via a linker. Exemplary linkers include a peptide of amino acid sequence as shown in any one of SEQ ID NOs. 47 to 50, or 144.

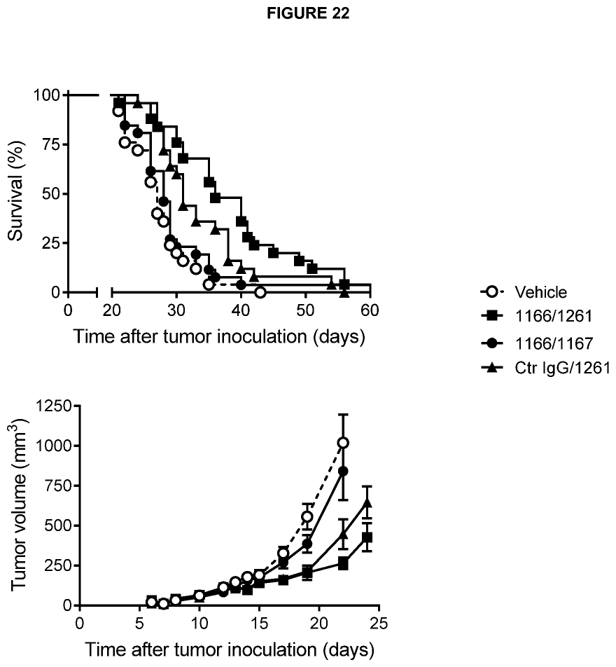

In the first scFv-IgG arrangement shown in FIG. 14 (top left of the Figure) the bispecific antibodies comprise two copies of a polypeptide chain which comprises the heavy chain variable sequence VH1 (checkered black and white), linked to a heavy chain constant sequence (Hc; filled grey), linked (optionally via a linker) to an scFv sequence consisting of the heavy chain variable sequence VH2 (filled black) and the light chain variable sequence VL2 (white with diagonal lines). This chain may be referred to as VH1-Hc-VH2/VL2 (ordered N terminus-C terminus). The bispecific antibody also comprises two copies of a smaller chain which comprises the light chain variable sequence VL1 (filled white) linked to a light chain constant sequence (Lc; filled grey), which may be referred to as VL1-Lc (ordered N terminus-C terminus). The alternative scFv-IgG arrangements shown in FIG. 14 also comprise two copies each of two different chains, which may be described in similar fashion. Thus, reading from left to right in the top row of FIG. 22, the second arrangement comprises two VH1-Hc chains and two VL1-Lc-VH2/VL2 chains. The third arrangement comprises two VH1/VH2-VH1-Hc chains and two VL1-Lc chains. The fourth arrangement comprises two VH1-Hc and two VH1/VH2-VL1-Lc chains.

In one embodiment, the bispecific polypeptides of the invention have the first scFv-IgG arrangement shown in FIG. 14 (top left of the Figure).

The present invention provides a polypeptide comprising or consisting of any of the amino acid sequences set out in Tables A-E, either alone or, preferably as part of a monospecific or bispecific antibody. In all of the sequences shown in Tables A-E, the sequences corresponding to heavy or light chain constant regions are exemplary and may be replaced with any other suitable heavy or light chain constant region sequence. Preferred heavy and light chain constant region sequences are those of SEQ ID NOs: 135, 136, 137, 138 and 139.

It will be appreciated that the invention also encompasses equivalent bispecific polypeptides in which a non-antibody polypeptide is used as a binding domain. In an embodiment of the invention part B1 of the polypeptide of the invention is an antibody, or antigen-binding fragment thereof, which typically comprises at least one heavy chain (H) and/or at least one light chain (L). Part B2 of the polypeptide of the invention may be attached to any part of B1, but may typically be attached to said at least one heavy chain (H) or at least one light chain (L), preferably at either the N or the C terminus. Part B2 of the polypeptide of the invention may be so attached either directly or indirectly via any suitable linking molecule (a linker).

Part B1 preferably comprises at least one heavy chain (H) and at least one light chain (L) and part B2 is preferably attached to the N or the C terminus of either said heavy chain (H) or said light chain (L). An exemplary antibody of B1 consists of two identical heavy chains (H) and two identical light chains (L). Such an antibody is typically arranged as two arms, each of which has one H and one L joined as a heterodimer, and the two arms are joined by disulfide bonds between the H chains. Thus, the antibody is effectively a homodimer formed of two H-L heterodimers. Part B2 of the polypeptide of the invention may be attached to both H chains or both L chains of such an antibody, or to just one H chain, or just one L chain.

The polypeptide of the invention may therefore alternatively be described as an anti-OX40 antibody, or an antigen binding fragment thereof, to which is attached at least one polypeptide binding domain specific for CTLA-4, which comprises or consists of the monomeric soluble extracellular domain of human wild-type CD86 or a variant thereof. The binding domains of B1 and B2 may be the only binding domains in the polypeptide of the invention.

The polypeptide of the invention may comprise a polypeptide arranged according to any one of the following formulae, written in the direction N-C:

L-(X)n-B2; (A)

B2-(X)n-L; (B)

B2-(X)n-H; and (C)

H—(X)n-B2; (D)

wherein H is the heavy chain of an antibody (i.e. of B1), L is the light chain of an antibody (i.e. of B1), X is a linker and n is 0 or 1. Where the linker (X) is a peptide, it typically has the amino acid sequence SGGGGSGGGGS (SEQ ID NO: 47), SGGGGSGGGGSAP (SEQ ID NO: 48), NFSQP (SEQ ID NO:49), KRTVA (SEQ ID NO: 50), GGGGSGGGGSGGGGS (SEQ ID NO: 144) or (SG)m, where m=1 to 7. Schematic representations of formulae (A) to (D) are shown in FIG. 1.

The present invention also provides a polypeptide which consists of a polypeptide arranged according to any of formulae (A) to (D). Said polypeptide may be provided as a monomer or may be present as a component of a multimeric protein, such as an antibody. Said polypeptide may be isolated. Examples of amino acid sequences of such polypeptides are shown in Table D. Exemplary nucleic acid sequences encoding each amino acid sequence are also shown.

Part B2 may be attached to any part of a polypeptide of the invention, or to a linker, by any suitable means. For example, the various parts of the polypeptide may be joined by chemical conjugation, such as with a peptide bond. Thus the polypeptide of the invention may comprise or consist of a fusion protein comprising B1 (or a component part thereof) and B2, optionally joined by a peptide linker. In such a fusion protein, the OX40-binding domain or domains of B1 and the CTLA-4-binding domain or domains of B2 may be the only binding domains.

Other methods for conjugating molecules to polypeptides are known in the art. For example, carbodiimide conjugation (see Bauminger & Wilchek, 1980, Methods Enzymol. 70:151-159; the disclosures of which are incorporated herein by reference) may be used to conjugate a variety of agents, including doxorubicin, to antibodies or peptides. The water-soluble carbodiimide, 1-ethyl-3-(3-dimethylaminopropyl) carbodiimide (EDC) is particularly useful for conjugating a functional moiety to a binding moiety. As a further example, conjugation may be achieved by sodium periodate oxidation followed by reductive alkylation of appropriate reactants, or by glutaraldehyde cross-linking. However, it is recognised that, regardless of which method is selected, a determination should preferably be made that parts B1 and B2 retain or substantially retain their target binding properties when present as parts of the polypeptide of the invention.

The same techniques may be used to link the polypeptide of the invention (directly or indirectly) to another molecule. The other molecule may be a therapeutic agent or a detectable label. Suitable therapeutic agents include a cytotoxic moiety or a drug.

A polypeptide of the invention may be provided in isolated or substantially isolated form. By substantially isolated, it is meant that there may be substantial, but not total, isolation of the polypeptide from any surrounding medium. The polypeptides may be mixed with carriers or diluents which will not interfere with their intended use and still be regarded as substantially isolated.

Exemplary polypeptides of the invention may comprise or consist of any one of the amino acid sequences shown in Table D. In one embodiment, the polypeptide comprises or consists of the amino acid sequence selected from within the group SEQ ID NOs 125 to 134, optionally wherein said polypeptide is a provided as a component part of an antibody.

Representative polynucleotides which encode examples of a heavy chain or light chain amino acid sequence of an antibody may comprise or consist of any one of the nucleotide sequences set out in Table B. Representative polynucleotides which encode the polypeptides shown in Table D may comprise or consist of the corresponding nucleotide sequences which are also shown in Table D (intron sequences are shown in lower case). Representative polynucleotides which encode examples of part B2 may comprise or consist of any one of SEQ ID NOS: 25 to 43 as shown in Table E. A suitable polynucleotide may alternatively be a variant of any of these sequences, as defined above.

In an embodiment of the invention, the bispecific polypeptide induces a synergistic activation of the host immune system against tumour cells, i.e. it is capable of inducing a synergistic increase in the intratumoural CD8/Treg ratio compared to the combined effect of the individual monospecific counterpart polypeptides (the CTLA-4 binding domain, or the individual OX40 monospecific antibody).

In an embodiment of the invention, the bispecific polypeptide is capable of inducing immunological memory in the host immune system against tumour cells.

By “immunological memory” we mean the ability of the immune system to quickly and specifically recognize an antigen in the body that it has previously encountered, such as a tumor antigen, and initiate a corresponding immune response.

Related Aspects of the Invention

A second aspect of the invention comprises a bispecific polypeptide according to the first aspect of the invention for use in a method for treating or preventing a disease or condition in an individual, as described above.

A third aspect of the invention is a method of treating or preventing a disease or condition in an individual, the method comprising administering to an individual a bispecific polypeptide according to the first or second aspects of the invention, as described above.

A fourth aspect of the invention is the use of a bispecific polypeptide according to the first aspect of the invention in the manufacture of a medicament.

One embodiment of the invention is a bispecific polypeptide according to the second aspect of the invention or a method according to third aspect of the invention wherein the disease or condition is cancer and optionally wherein the individual is human, or the use according to the fourth aspect of the invention wherein the medicament is for the treatment is cancer, optionally wherein the individual is human.

In a further embodiment, the method comprises administering the bispecific antibody systemically or locally, such as at the site of a tumour or into a tumour draining lymph node, as described above.

The cancer may be prostate cancer, breast cancer, colorectal cancer, pancreatic cancer, ovarian cancer, lung cancer, cervical cancer, rhabdomyosarcoma, neuroblastoma, multiple myeloma, leukemia, acute lymphoblastic leukemia, melanoma, bladder cancer, gastric cancer, head and neck cancer, liver cancer, skin cancer, lymphoma or glioblastoma.

A fifth aspect of the invention is a polynucleotide encoding at least one polypeptide chain of a bispecific polypeptide according to the first or second aspects of the invention, as described above.

A sixth aspect of the invention is a composition comprising a bispecific polypeptide according to the first or second aspects of the invention and at least one pharmaceutically acceptable diluent or carrier.

In one embodiment of the invention a polypeptide according to either the first or second aspect of the embodiment is conjugated to an additional therapeutic moiety.

The pharmaceutical compositions will be administered to a patient in a pharmaceutically effective dose. A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art. The administration of the pharmaceutically effective dose can be carried out both by single administration in the form of an individual dose unit or else several smaller dose units and also by multiple administrations of subdivided doses at specific intervals. Alternatively, the does may be provided as a continuous infusion over a prolonged period.

Particularly preferred compositions are formulated for systemic administration.

The composition may preferably be formulated for sustained release over a period of time. Thus the composition may be provided in or as part of a matrix facilitating sustained release. Preferred sustained release matrices may comprise a montanide or γ-polyglutamic acid (PGA) nanoparticles.

The antibody polypeptides can be formulated at various concentrations, depending on the efficacy/toxicity of the polypeptide being used. For example, the formulation may comprise the active antibody polypeptide at a concentration of between 0.1 μM and 1 mM, more preferably between 1 μM and 500 μM, between 500 μM and 1 mM, between 300 μM and 700 μM, between 1 μM and 100 μM, between 100 μM and 200 μM, between 200 μM and 300 μM, between 300 μM and 400 μM, between 400 μM and 500 μM, between 500 μM and 600 μM, between 600 μM and 700 μM, between 800 μM and 900 μM or between 900 μM and 1 mM. Typically, the formulation comprises the active antibody polypeptide at a concentration of between 300 μM and 700 μM.

Typically, the therapeutic dose of the antibody polypeptide (with or without a therapeutic moiety) in a human patient will be in the range of 100 μg to 700 mg per administration (based on a body weight of 70 kg). For example, the maximum therapeutic dose may be in the range of 0.1 to 10 mg/kg per administration, e.g. between 0.1 and 5 mg/kg or between 1 and 5 mg/kg or between 0.1 and 2 mg/kg. It will be appreciated that such a dose may be administered at different intervals, as determined by the oncologist/physician; for example, a dose may be administered daily, twice-weekly, weekly, bi-weekly or monthly.

It will be appreciated by persons skilled in the art that the pharmaceutical compositions of the invention may be administered alone or in combination with other therapeutic agents used in the treatment of cancers, such as antimetabolites, alkylating agents, anthracyclines and other cytotoxic antibiotics, vinca alkyloids, etoposide, platinum compounds, taxanes, topoisomerase I inhibitors, other cytostatic drugs, antiproliferative immunosuppressants, corticosteroids, sex hormones and hormone antagonists, and other therapeutic antibodies (such as antibodies against a tumour-associated antigen or an immune checkpoint modulator).

For example, the pharmaceutical compositions of the invention may be administered in combination with an immunotherapeutic agent that binds a target selected from the group consisting of PD-1/PD-L1, CD137, CD40, GITR, LAG3, TIM3, CD27 and KIR.

Thus, the invention encompasses combination therapies comprising a bispecific polypeptide of the invention together with a further immunotherapeutic agent, effective in the treatment of cancer, which specifically binds to an immune checkpoint molecule. It will be appreciated that the therapeutic benefit of the further immunotherapeutic agent may be mediated by attenuating the function of an inhibitory immune checkpoint molecule and/or by activating the function of a stimulatory immune checkpoint or co-stimulatory molecule.

In one embodiment, the further immunotherapeutic agent is selected from the group consisting of:
(a) an immunotherapeutic agent that inhibits the function of PD-1 and/or PD-L1;
(b) an immunotherapeutic agent that activates the function of CD137; and
(c) an immunotherapeutic agent that activates the function of CD40.

Thus, the further immunotherapeutic agent may be a PD1 inhibitor, such as an anti-PD1 antibody, or antigen-binding fragment thereof capable of inhibiting PD1 function (for example, Nivolumab, Pembrolizumab, Lambrolizumab, PDR-001, MEDI-0680 and AMP-224). Alternatively, the PD1 inhibitor may comprise or consist of an anti-PD-L1 antibody, or antigen-binding fragment thereof capable of inhibiting PD1 function (for example, Durvalumab, Atezolizumab, Avelumab and MDX-1105).

In a further embodiment, the further immunotherapeutic agent activates CD137, such as an agonistic anti-CD137 antibody or antigen-binding portion thereof.

In a further embodiment, the further immunotherapeutic agent activates CD40, such as an agonistic anti-CD40 antibody or antigen-binding portion thereof.

It will be appreciated by persons skilled in the art that the presence of the two active agents (as detailed above) may provide a synergistic benefit in the treatment of a tumour in a subject. By “synergistic” we include that the therapeutic effect of the two agents in combination (e.g. as determined by reference to the rate of growth or the size of the tumour) is greater than the additive therapeutic effect of the two agents administered on their own. Such synergism can be identified by testing the active agents, alone and in combination, in a relevant cell line model of the solid tumour.

Also within the scope of the present invention are kits comprising polypeptides or other compositions of the invention and instructions for use. The kit may further contain one or more additional reagents, such as an additional therapeutic or prophylactic agent as discussed above.

Preferred, non-limiting examples which embody certain aspects of the invention will now be described, with reference to the following figures:

FIG. 1 shows a schematic representation of the structure of exemplary arrangements for the bispecific polypeptides of the invention. Anti-OX40 antibody variable domains are filled in black; constant domains in white. CTLA-A binding domains are shaded with diagonal lines.

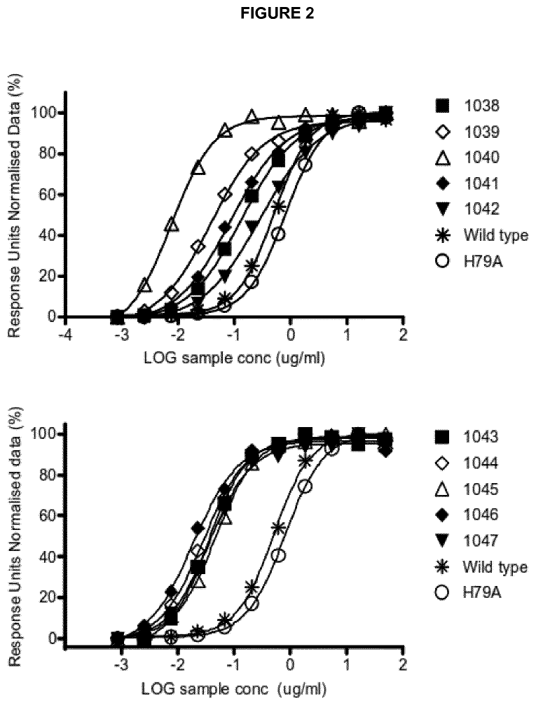

FIG. 2 shows the CTLA-4 binding properties of CTLA-4 binding domains of polypeptides the invention as determined by an ELISA binding assay.

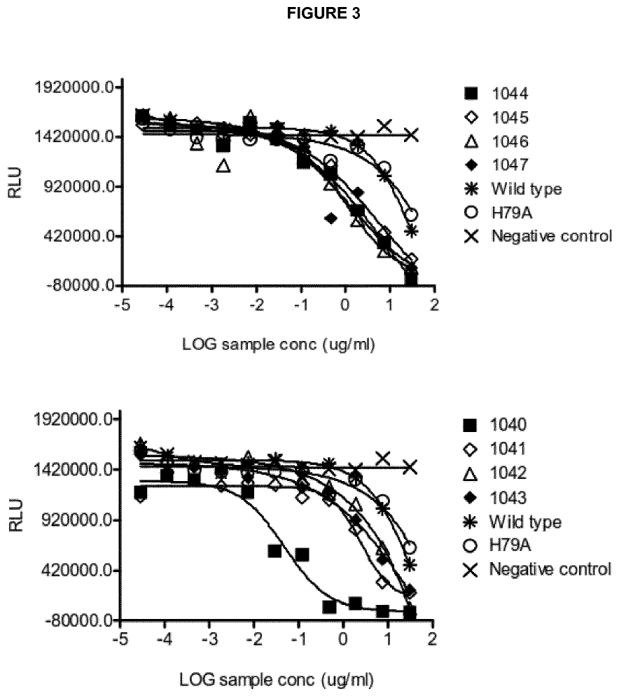

FIG. 3 shows the CTLA-4 binding properties of CTLA-4 binding domains of polypeptides of the invention as determined by an ELISA inhibition assay.

FIG. 4 provides a schematic representation of human wild-type CD86 amino acid sequences disclosed herein. (A) is the amino acid sequence of the monomeric soluble extracellular domain of human CD86 without N-terminal signal sequence (SEQ ID NO: 3); (B) is the amino acid sequence of the monomeric extracellular and transmembrane domains of human wildtype CD86, including N-terminal signal sequence (SEQ ID NO: 4); (C) is the full length amino acid sequence of human CD86 (Genbank ABK41931.1; SEQ ID NO: 44). The sequence in A may optionally lack Alanine and Proline at the N terminus, i.e. positions 24 and 25, shown in bold. Signal sequences in B and C are underlined. Numbering of amino acid positions is based on SEQ ID NOs: 4 and 44, starting from the N terminus.

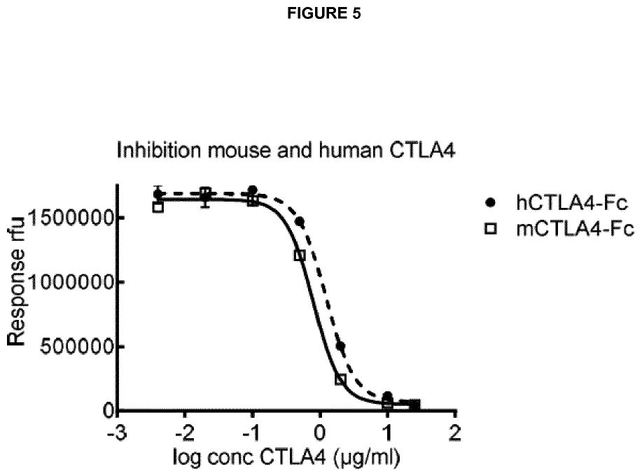

FIG. 5 shows the results of an inhibition ELISA demonstrating that a CTLA-4 binding domains of polypeptides of the invention has binding affinity of a similar magnitude for both human and murine CTLA-4.

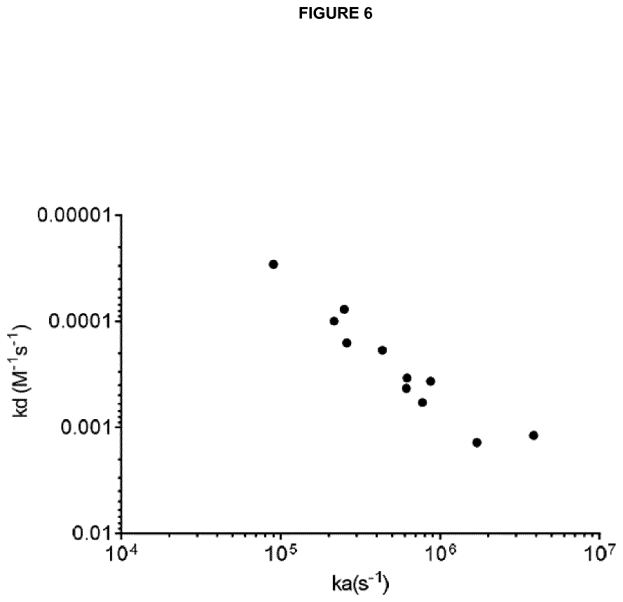

FIG. 6 is a plot of dissociation rate constant versus association rate constant for exemplary anti-OX40 antibodies, as determined by surface plasmon resonance.

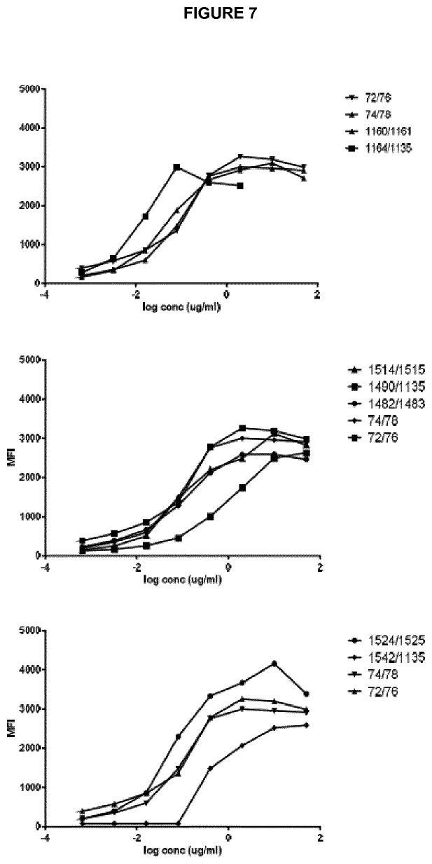

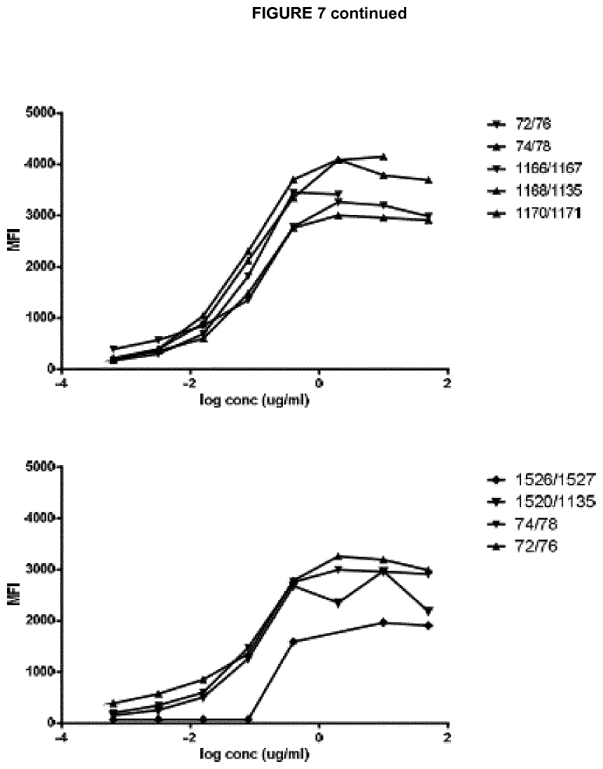

FIG. 7 shows binding of exemplary anti-OX40 antibodies to human OX40 overexpressed on CHO cells, measured by flow cytometry.

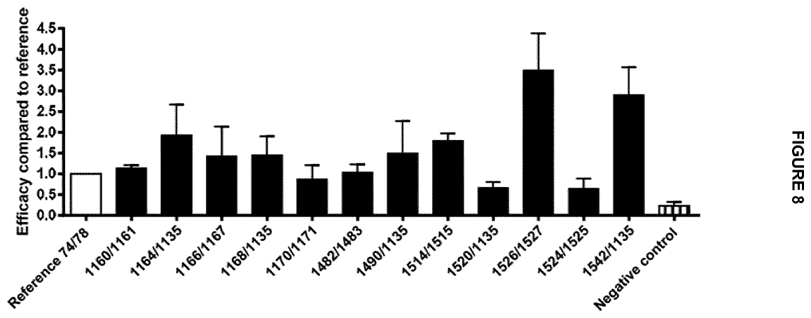

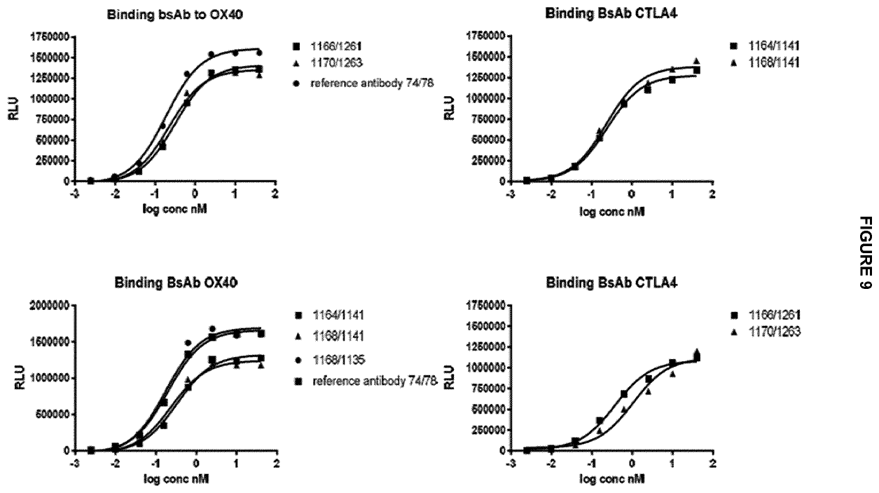

FIG. 8 shows the level of IL-2 production by T cells when incubated in vitro with different exemplary anti-OX40 antibodies. The y-axis is the ratio of the top value of IL-2 production by a tested antibody/the top value of a reference antibody. Mean and SEM values from at least 4 donors are shown.

FIG. 9 shows results of an ELISA assay for binding of exemplary bispecific molecules to individual targets OX40 and CTLA4.

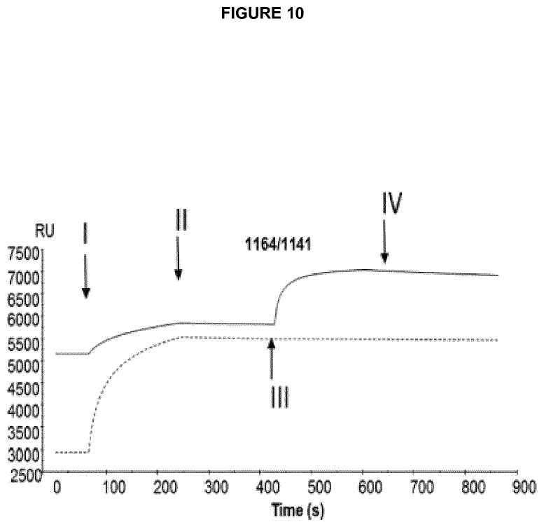

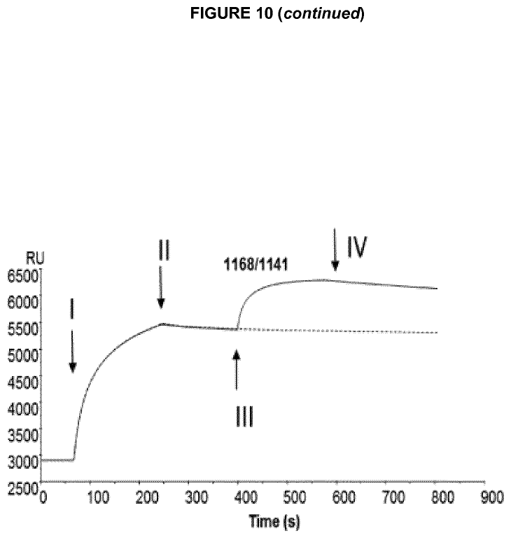

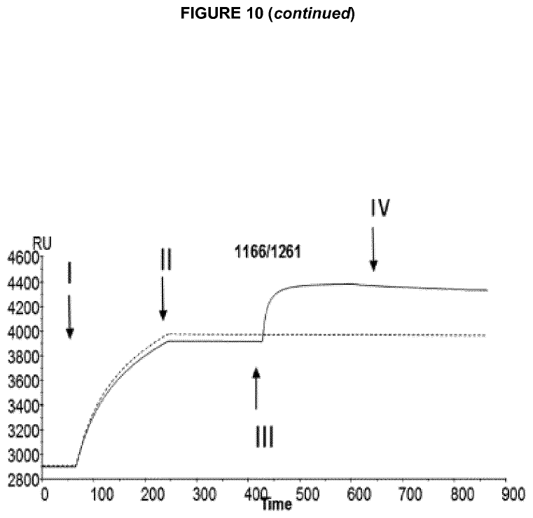

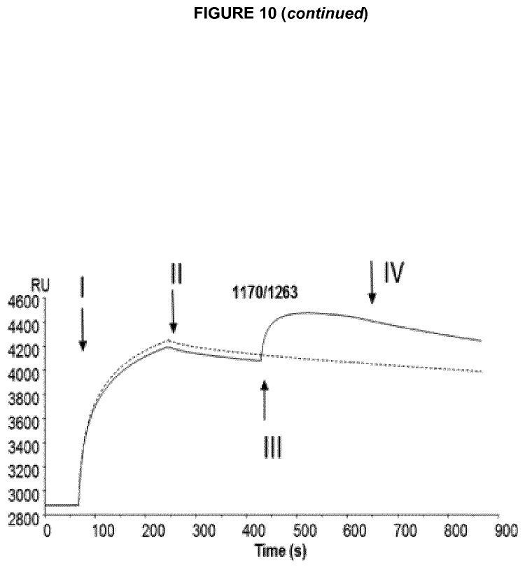

FIG. 10 shows results of surface plasmon resonance analysis of binding of exemplary bispecific molecules to both OX40 and CTLA4. The different bispecific antibodies were passed over the sensor (start indicated by I)). At near saturation of the surface, buffer was applied (II) and subsequently CTLA-4 (III) was passed over the sensor surface generating a second association phase, represented by the full line. After three minutes, buffer (IV) was applied, and the following dissociation phase reflects dissociation of both CTLA-4 and OX40 Ab. As a control, only buffer, with no CTLA-4 was added, represented by the dotted line.

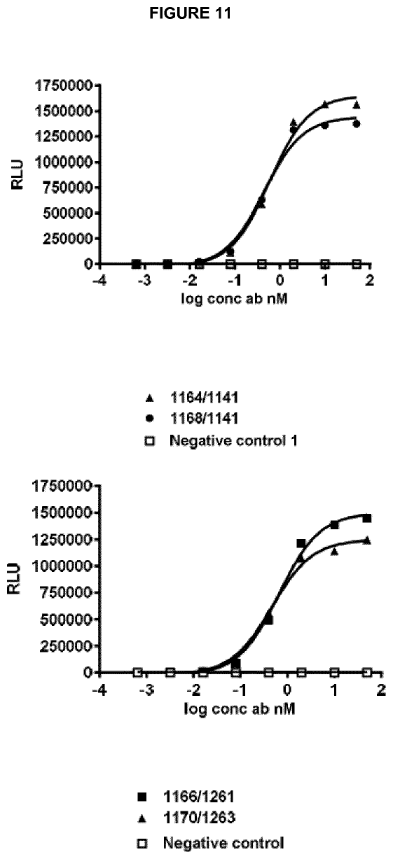

FIG. 11 shows results of an ELISA assay showing binding of exemplary bispecific molecules to both OX40 and CTLA-4 simultaneously.

FIG. 12 shows the level of IL-2 production by T cells when incubated in vitro with different exemplary bispecific molecules in a titration series: A) 1164/1141 and 1166/1141 B) 1168/1141 and 1170/1263 C) 1514/1581 and 1520/1141 D) 1526/1585 and 1542/1141 or a combination of the two corresponding monospecific antibodies for each target (monoclonal OX40 antibodies or the CTLA-4-binding domain coupled to an isotype IgG antibody: 1756/1757). The assay was performed in U-shaped non-tissue cultured treated 96-well plates coated with CD3 (UCHT1) and CTLA-4 (Orencia). Mean out of 4 donors is presented.

FIG. 13 shows the level of IL-2 production by T cells when incubated in vitro with different exemplary bispecific molecules at 1.49 nM or a combination of the corresponding monospecific antibodies for each target (a-OX40 mAbs or the CTLA-4-domain coupled to an isotype antibody: 1756/1757). The assay was performed in U-shaped non-tissue cultured treated 96-well plates coated with anti-CD3 (UCHT1) with or without CTLA-4 (Orencia), indicated by + or −. Mean and SD out of 4 donors is presented.

FIG. 14 shows a schematic representation of the structure of exemplary formats for bispecific antibodies of the invention. In each format, the constant regions are shown as filled light grey; variable heavy chain regions VH1 are shown as checkered black and white; variable light chain regions VL1 are shown as filled white; variable heavy chain regions VH2 are shown as filled black; and variable light chain regions VL2 are shown as white with diagonal lines. OX40 binding domains (binding domain 1) are typically represented as a pair of a checkered black and white domain with a filled white domain (VH1/VL1); CD137 binding domains (binding domain 2) are typically represented as a pair of a filled black domain and a white domain with diagonal lines (VH2/VL2). However, in all of the formats shown, it will be appreciated that binding domains 1 and 2 may be switched. That is, an OX40 binding domain may occur in a position shown in this figure for a CD137 domain, and vice versa. Furthermore, binding domain 2 may occur in different variable heavy and light chain orders, i.e. either in VH2/VL2 or VL2/VH2 order.

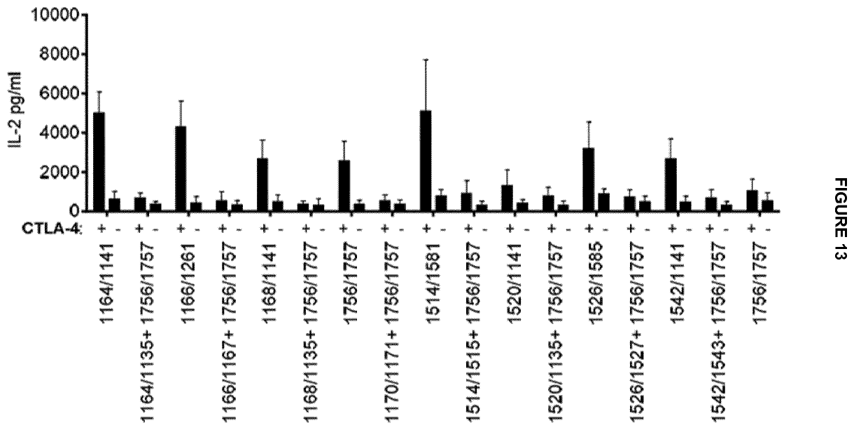

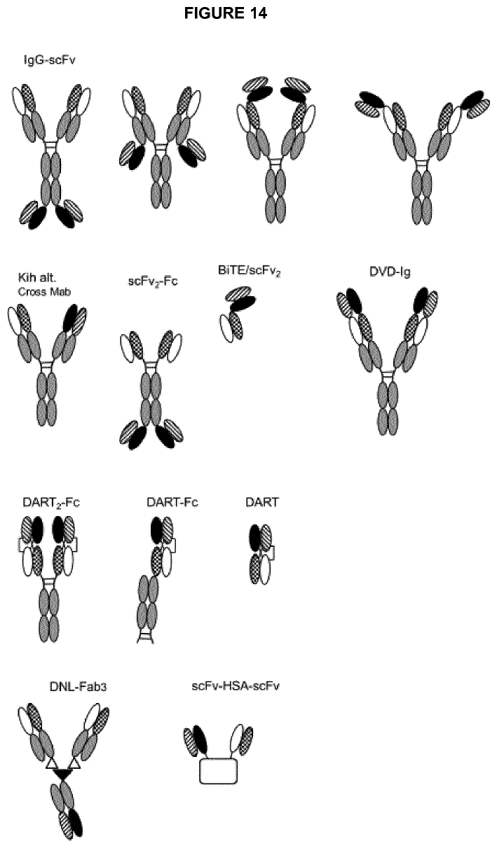

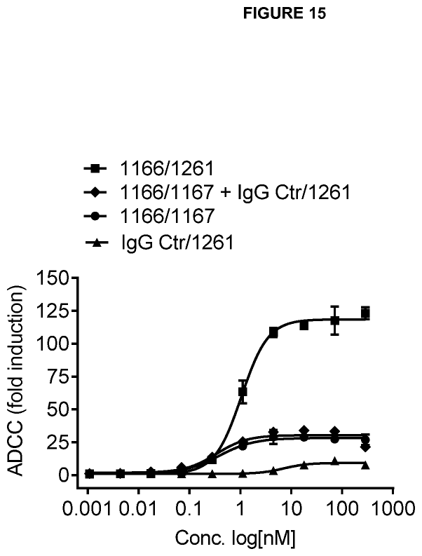

FIG. 15 shows induction of ADCC at different concentration by monospecific CTLA-4 (Control IgG with CTLA-4 binding part, i.e. domain) and OX40 (1166/1167) binding molecules, alone and in combination, compared to ADCC induced by an exemplary bispecific antibody targeting CTLA-4 and OX40.

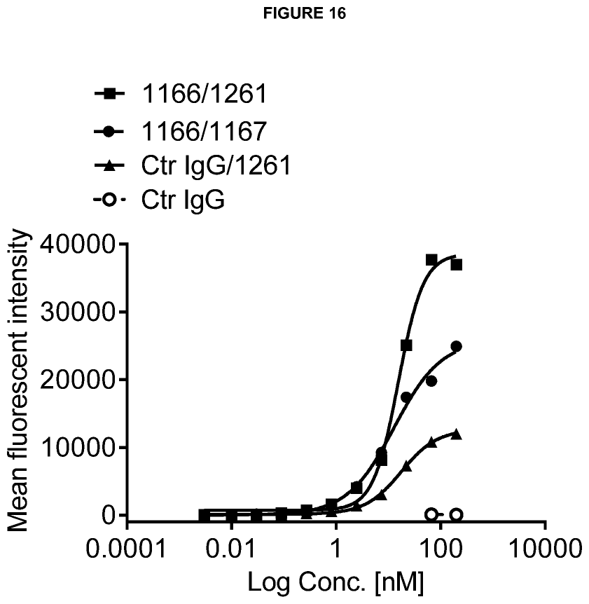

FIG. 16. CHO cells expressing both CTLA-4 and OX40 were stained with decreasing concentrations of 1166/1261, or the two monospecific binders 1166/1167 (OX40 specific monoclonal antibody) and control IgG with CTLA-4 binding part (monospecific CTLA4 binding IgG fusion protein) (200 nM-0.0034 nM), followed by PE-conjugated anti-human IgG. Fluorescence was detected using flow cytometry. ‘Ctr IgG’ is a negative isotype control.

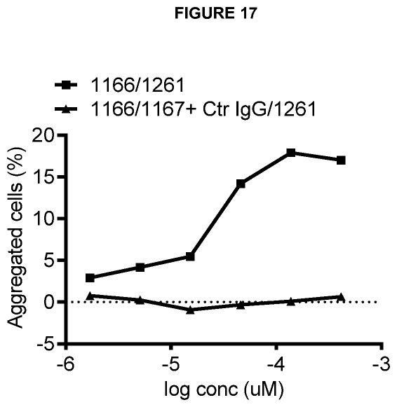

FIG. 17. HEK-CTLA4 and CHO-OX40 were stained with PKH26 and PKH67 respectively and incubated with 1166/1261 or a combination of the two monospecific OX40 and CTLA-4 binding molecules (1166/1167 and Control IgG with CTLA-4 binding part). The percentage of double-positive/aggregated cells were quantified using flow cytometry (representative experiment).

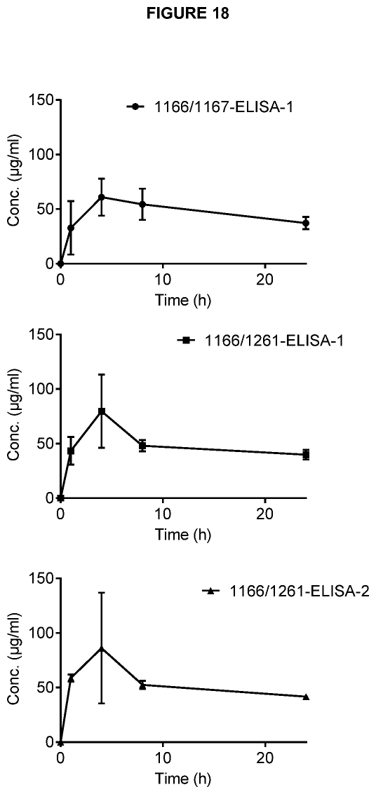

FIG. 18. Plasma levels of the bispecific OX40-CTLA-4 antibody 1166/1261 and the monospecific OX40 antibody 1166/1167 measured at different time points following administration. Two different ELISA methods were used, ELISA-1, where OX40 was coated on the wells and anti-Fc was used to detect binding and ELISA-2, where OX40 was coated on the wells and biotinylated CTLA-4 was used for detection.

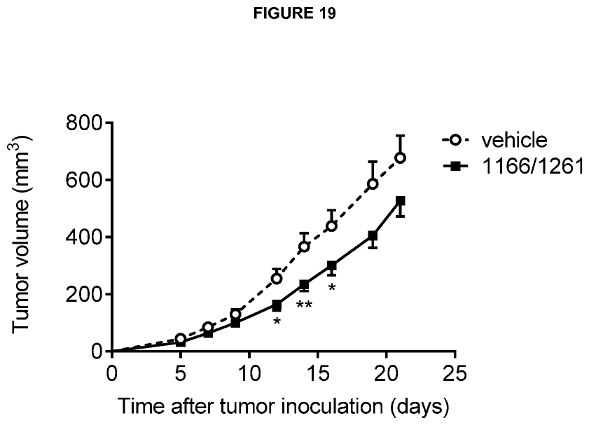

FIG. 19. HT-29 colon carcinoma cells (4×10⁶) were inoculated subcutaneously to the right hind flank/back at day 0. Human PBMC cells (7×10⁶) were administered intraperitoneally on the same day. The treatments were done by intraperitoneal injections (667 nmol/dose) on days 6, 13, and 20. N(mice)=5/donor, n(donor=4), pooled data from HT29 responders.

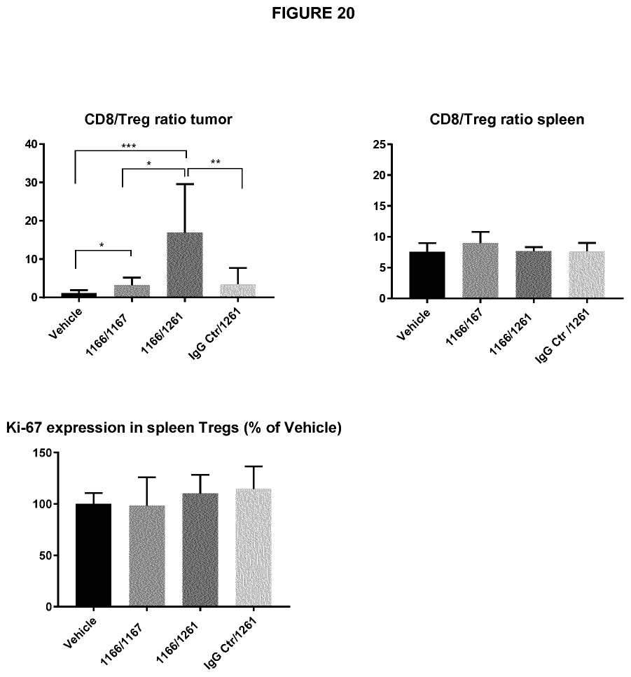

FIG. 20 shows the pharmacodynamics effects of a bispecific OX40-CTLA-4 antibody investigated in hOX40tg mice using the MC38 colon carcinoma model. Pooled data from two independent experiments demonstrated statistically significant effect on intratumoural CD8/Treg ratio with bispecific antibody compared to both monospecific counterparts.

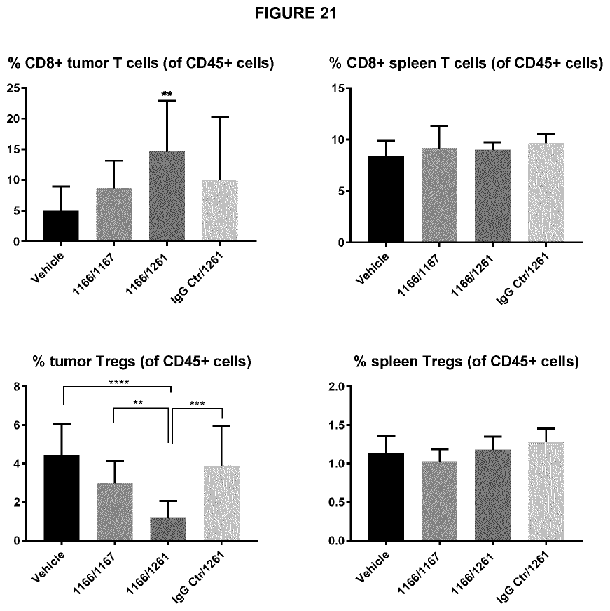

FIG. 21 shows the relative levels of the different T cell populations in spleen and tumour tissues of in hOX40tg mice using the MC38 colon carcinoma model. The effect of administering the bispecific antibody is compared to monospecific counterparts. The bispecific antibody reduces intratumoural Tregs but does not affect systemic T-cells.

FIG. 22 shows the anti-tumour effect of bispecific OX40-CTLA-4 antibody, investigated in transgenic mice for human OX40 and using MC38 colon carcinoma model. The bispecific antibody demonstrated statistically significant effects on tumour volume inhibition and increased survival. The anti-tumour effect was stronger in terms of survival and tumour growth inhibition than the monospecific control antibodies.

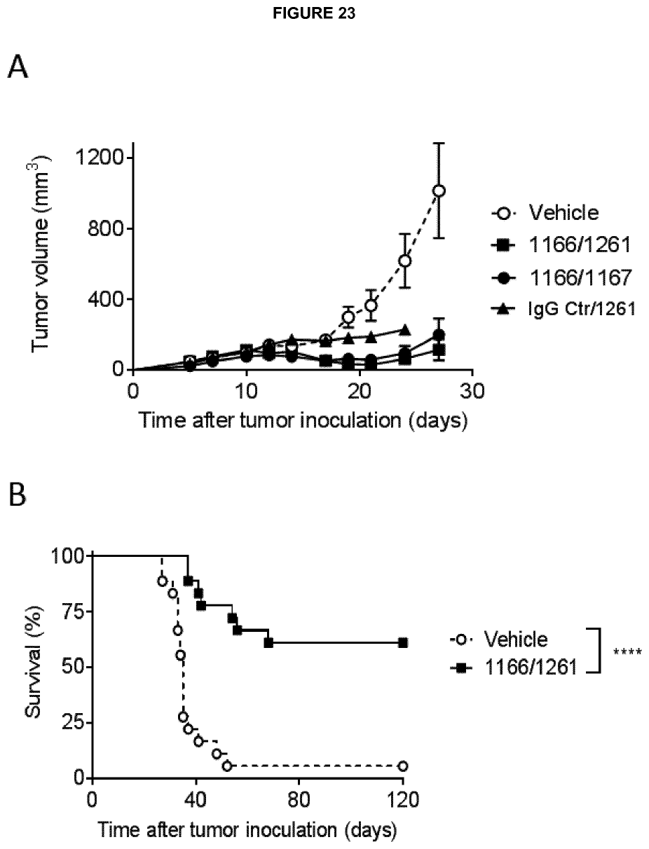

FIG. 23 shows anti-tumour efficacies induced by bispecific OX40-CTLA antibody 1166/1261. The anti-tumour efficacy was investigated in MB49 bladder carcinoma model using hOX40 transgenic mice. Intraperitoneal treatments on mice bearing subcutaneous MB49 tumours were done intraperitoneally on days 7, 10 and, 13. A) Tumour volume inhibition by 1166/1261 or the monoclonal counterparts. B) Increased survival induced by 1166/1261. The tumour volume graph is exemplary of several performed, tumour volume mean+/−SEM, n=10. Kaplan-Meyer survival, pooled data from two individual experiments, n=18.

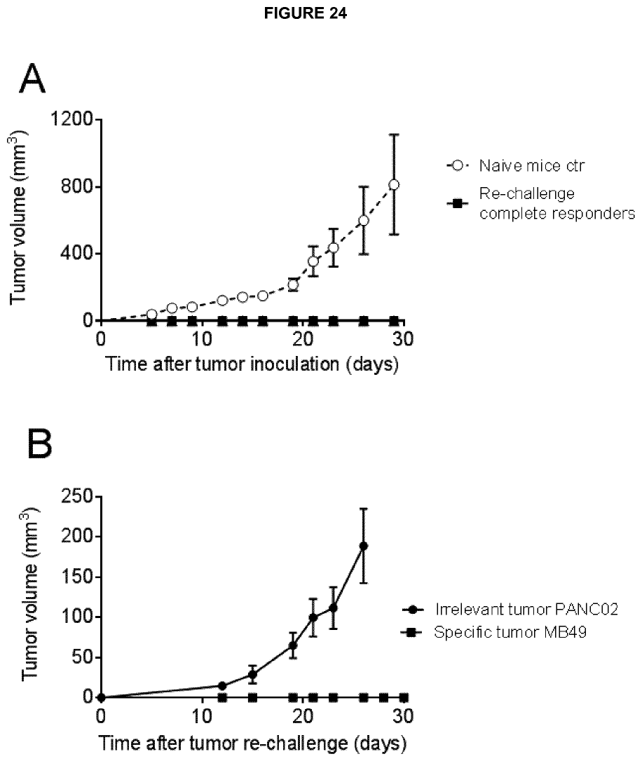

FIG. 24 shows the immunological memory, which was demonstrated by re-challenging complete responders with the same tumour as specific tumour or with an irrelevant tumour. A) Re-challenge of mice with the same tumour. Naïve mice were used as a control. (n=5). B) Re-challenge of mice in a twin tumour model with one specific tumour MB49 in one flank and one irrelevant tumour PANC02 in the other flank. Exemplary experiment, graph shows mean+/−SEM, n=6.

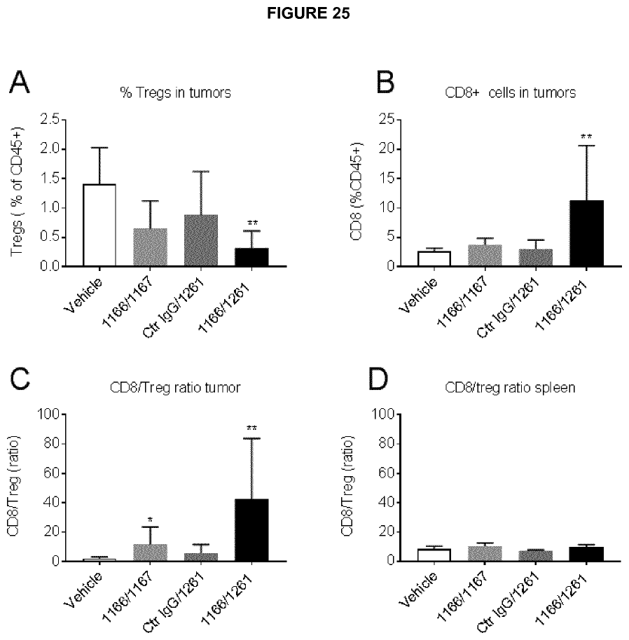

FIG. 25 shows anti-tumour effects of bispecific antibodies on Tregs. Mice bearing subcutaneous MB49 bladder cancer were treated with intraperitoneal injections of 1166/1261 or with the monoclonal counterparts (1.33 μmol) on days 10, 13 and, 16. Twenty-four hours after the last injection, the tumours and spleens were harvested, and stained for Tregs and effector cells. A) Percent Tregs (of CD45) in tumours B) CD8 cells (of CD45) in tumours C) CD8/Treg ratio in tumours and D) CD8/Treg ratio in spleens. The graphs show mean+SD.

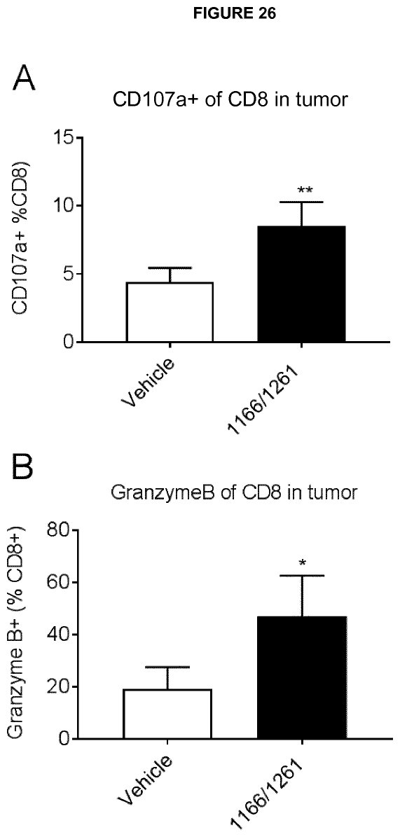

FIG. 26 shows anti-tumour effects of bispecific antibodies on MC38 colon carcinoma. Mice bearing subcutaneous MC38 colon carcinoma were treated with intraperitoneal injections with 1166/1261 on days 10, 13 and, 16. Twenty-four hours after the last injection, the tumours were harvested, and stained for effector cells and activation markers. A) Percent CD107+CD8 cells in tumours (B) Percent GranzymeB+CD8 cells in tumours. The graphs show mean+SD.

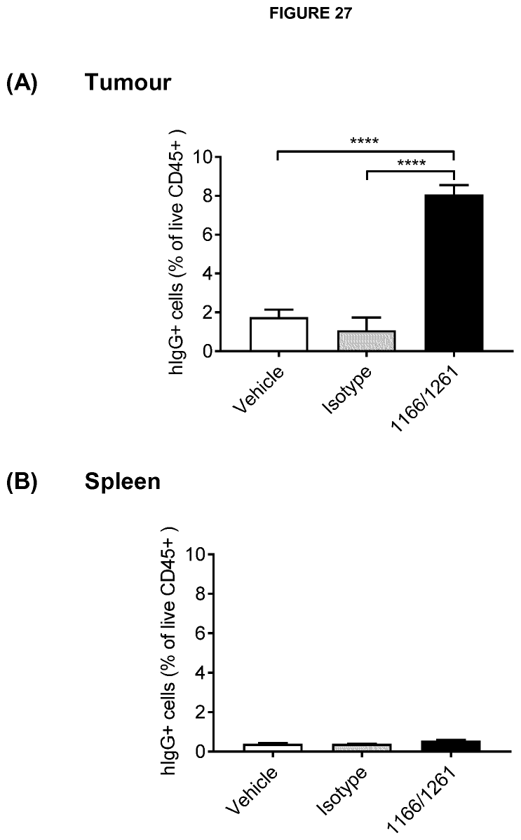

FIG. 27 shows tumour localization in hOX40tg mice with MC38 colon carcinoma. Mice bearing subcutaneous MC38 colon carcinoma were treated intraperitoneally with vehicle, IgG1 isotype control or 1166/1261 on day 17. Twenty-four hours after the injection, the tumors and spleens were harvested, stained with a viability marker and antibodies against CD45 and hlgG, followed by flow cytometry analysis. The percentage of hIgG⁺ cells out of live CD45⁺ cells in (A) tumors and (B) spleens were compared between the different groups. The graphs show mean+SEM.

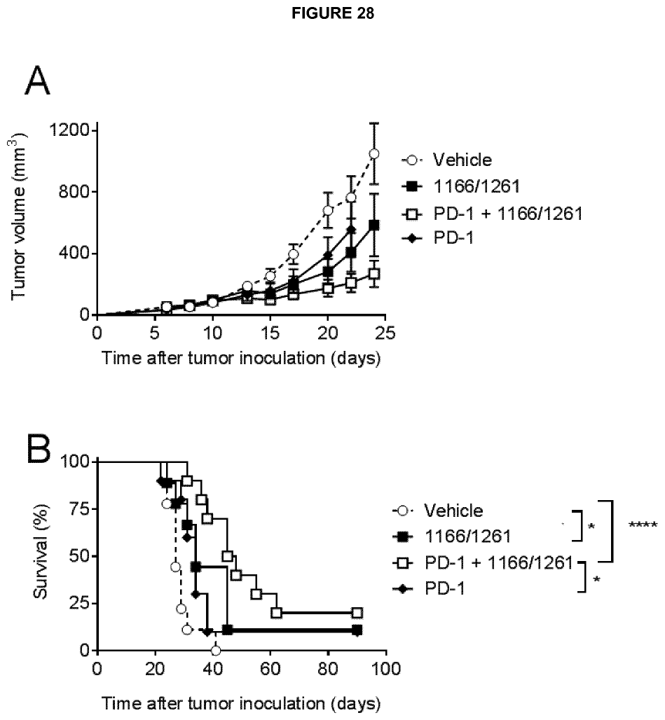

FIG. 28 shows combinatorial effects of PD-1 antibody with 1166/1261. The anti-tumour effects of 1166/1261 with or without PD-1 treatment was investigated in MC38 colon carcinoma model. Intraperitoneal treatments (1166/1261, 1.33 μmol or 250 μg PD-1) were done intraperitoneally on days 7, 10 and, 13. A) Tumour volume inhibition by 1166/1261 with or without PD-1 combination. B) Increased survival induced by 1166/1261 with or without PD-1 treatment. The tumour volume graph is exemplary graph and presents mean tumour volume+/−SEM, or Kaplan-Meyer survival, n=9-10.

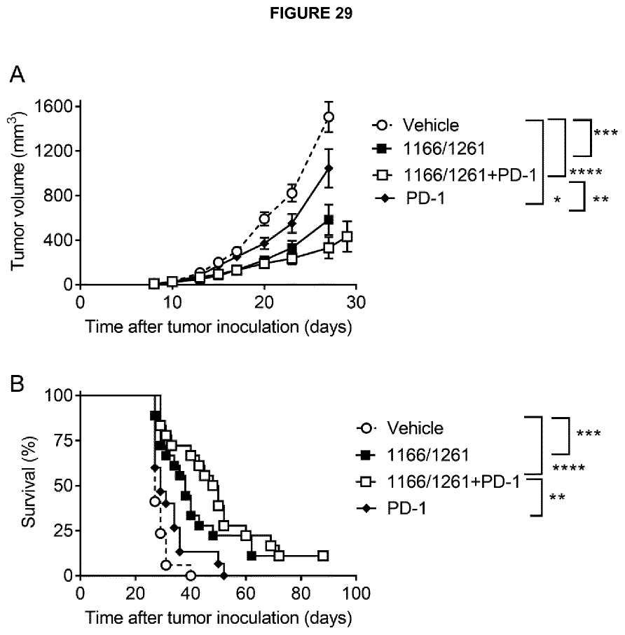

FIG. 29 shows combinatorial effects of PD-1 antibody with 1166/1261. The anti-tumour effects of 1166/1261 with or without PD-1 treatment was investigated in CT26 colon carcinoma model. Intraperitoneal treatments were done on days 7, 10 and, 13. A) Tumour volume inhibition by 1166/1261 with or without PD-1). B) Kaplan-Meyer survival by 1166/1261 with or without PD-1 treatment. The graphs shown two independent experiments pooled together, tumour volume mean+/−SEM, n=18.

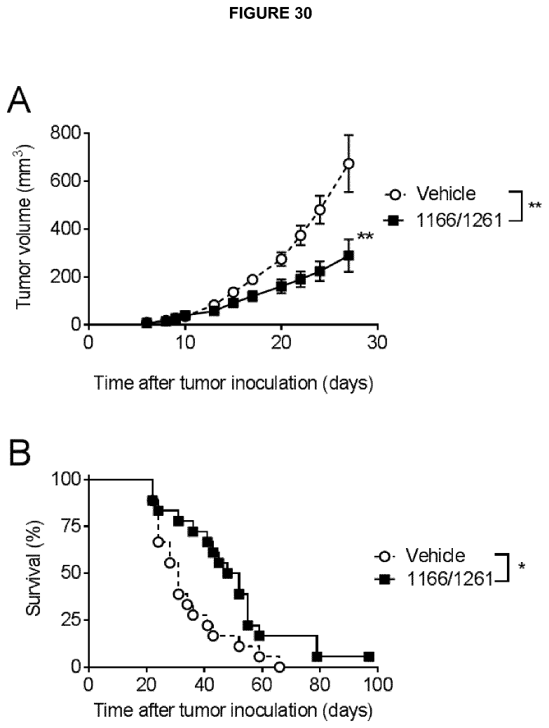

FIG. 30 shows anti-tumour effect of 1166/1261 in pancreatic cancer. The anti-tumour effect of bispecific OX40-CTLA-4 antibody 1166/1261 was investigated in transgenic mice for human OX40 using PANC02 pancreatic cancer. The intraperitoneal treatments were done on days 7, 10 and 13. A) Tumour volume inhibition. B) Increased survival. Graphs show mean tumour volume+/−SEM, or Kaplan Meyer survival n=18.

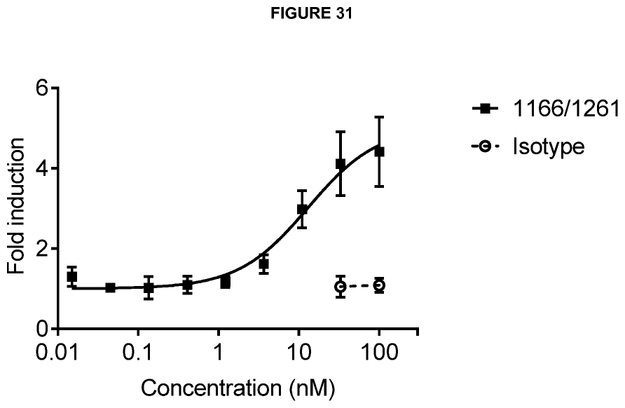

FIG. 31 shows the ability of 1166/1261 and isotype control to induce T cell activation by blocking CTLA-4 on Jurkat reporter cells in a CTLA-4 Blockade Reporter assay. Compiled data from two experiments.

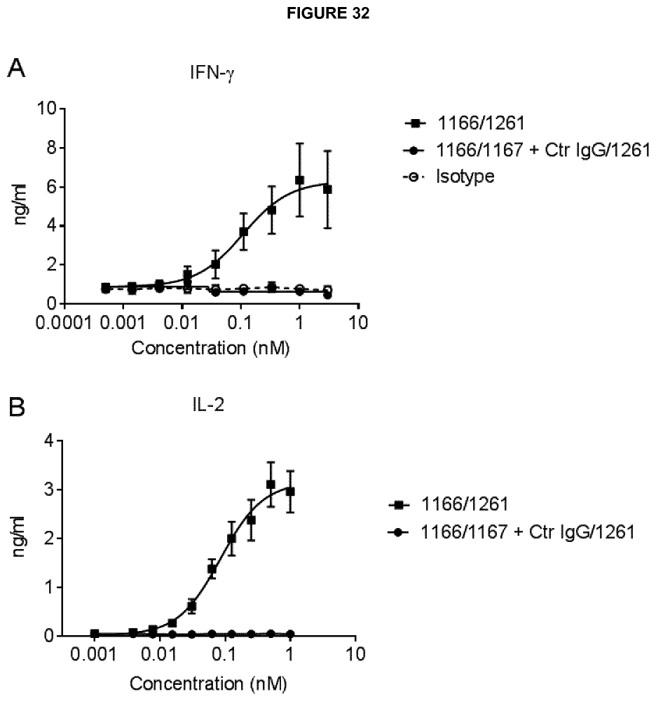

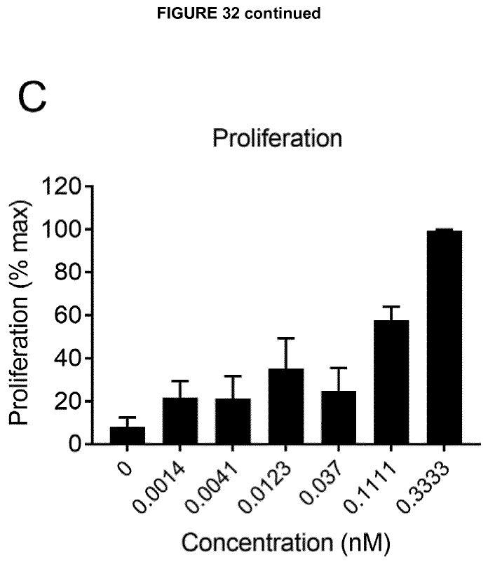

FIG. 32 shows T cell activation. A) IFN-γ production following stimulation of human CD3⁺ T cells with 1166/1261, the combination of monospecific antibodies or isotype control. The experiment was performed in plates coated with CTLA-4 and αCD3. Compiled data from 4 donors. B) IL-2 release by CD4+ T cells stimulated with 1166/1261 or combination of monospecific antibodies in the presence of CTLA-4-expressing HEK cells and αCD3 beads. Compiled data from 6 donors. C) Proliferation of CD4+ T cells in response to 1166/1261 or isotype control. Compiled data from 6 donors.

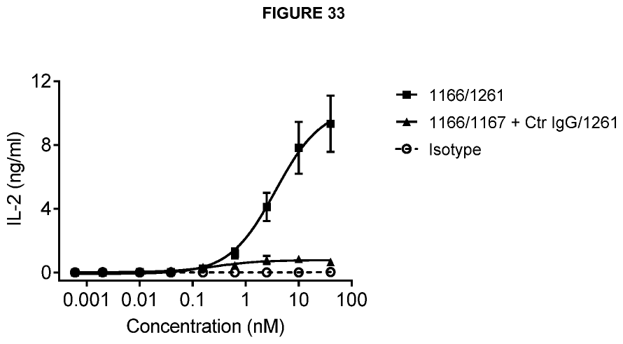

FIG. 33 shows T cell activation. IL-2 release by CD4+ T cells stimulated with 1166/1261 or combination of monospecific antibodies in the presence of CD64-expressing CHO cells and αCD3 beads. Compiled data from 8 donors.

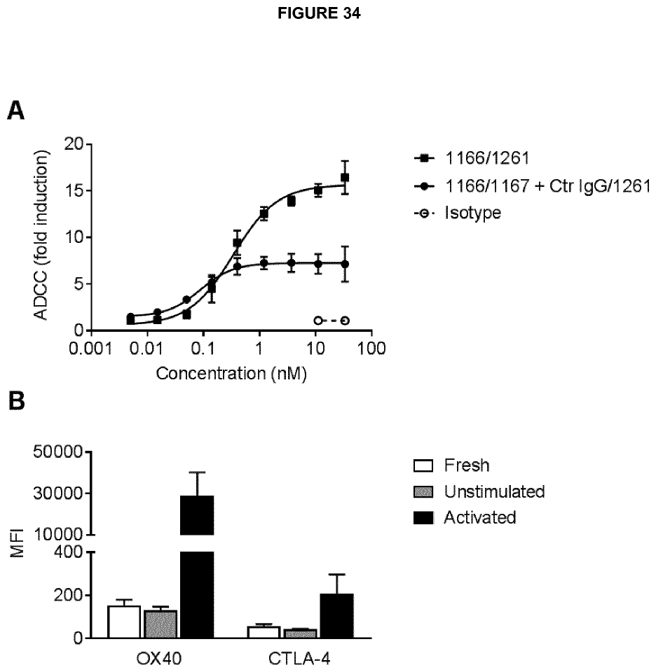

FIG. 34 shows ADCC induction by 1166/1261. A) Activation of FcγRIIIa (V158) effector cells in response to 1166/1261, mixture of the monoclonal counterpart or isotype control. Purified Tregs activated for 48 h with αCD3/αCD28 beads were used as target cells. Data is presented as fold induction over medium control. B) Expression of OX40 and CTLA-4 was determined by flow cytometry on Tregs before and after activation. The mean of three donors is shown.

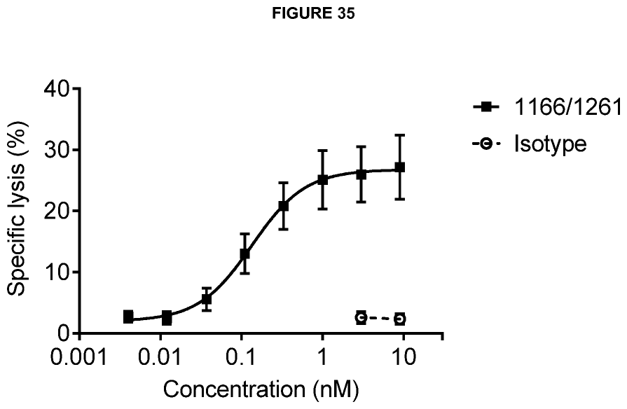

FIG. 35 shows ADCC in response to 1166/1261. Purified Tregs activated for 48 h with αCD3/αCD28 beads were used as target cells and allogeneic NK cells as effector cells. Effector and target cells were cultured at a 15:1 ratio in the presence of 1166/1261 or isotype control. After 4 h, LDH release was measured. Compiled data from 7 donors.

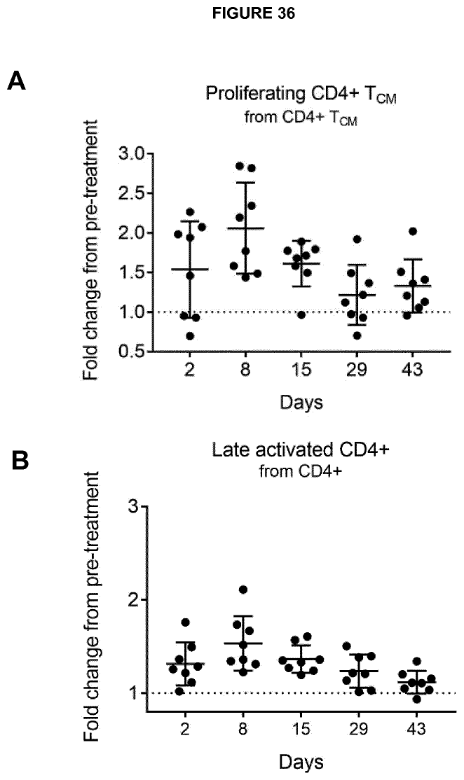

FIG. 36 shows activation of cynomolgus T cells activation by 1166/1261 treatment. A) Proliferation of central memory CD4+ cells B) Late activation of CD4+ T cells.

### DESCRIPTION OF THE SEQUENCES

SEQ ID NO: 1 is the amino acid sequence of human CTLA-4 (corresponding to GenBank: AAD00698.1)

SEQ ID NO: 2 is the amino acid sequence of human CD28 (corresponding to GenBank: AAA51944.1)

SEQ ID NO: 3 is the amino acid sequence of the monomeric extracellular domain of human wildtype CD86, excluding a 23-amino acid signal sequence from the N terminus.

SEQ ID NO: 4 is the amino acid sequence of the monomeric extracellular and transmembrane domains of human wildtype CD86, including N-terminal signal sequence (see FIG. 4). All numbering of amino acid positions herein is based on the positions in SEQ ID NO: 4 starting from the N terminus. Thus, the Alanine at the N terminus of SEQ ID NO: 3 is numbered 24.

SEQ ID NO: 5 is the amino acid sequence of a mutant form of the extracellular domain of human CD86 disclosed in Peach et al (Journal of Biological Chemistry 1995, vol 270(36), 21181-21187). H at position 79 of the wild type sequence is substituted with A in the corresponding position for the sequence of SEQ ID NO: 5. This change is referred to herein as H79A. Equivalent nomenclature is used throughout for other amino acid substitutions referred to herein. Numbering of positions is based on SEQ ID NO: 4 as outlined above.

SEQ ID NOs: 6 to 24 are the amino acid sequences of specific proteins of the invention.

SEQ ID NOs: 25 to 43 are nucleotide sequences encoding the amino acid sequences of each of SEQ ID NOs 6 to 24, respectively

SEQ ID NO: 44 is the full length amino acid sequence of human CD86 (corresponding to GenBank: ABK41931.1)

SEQ ID NO: 45 is the amino acid sequence of murine CTLA-4 (corresponding to UniProtKB/Swiss-Prot: P09793.1).

SEQ ID NO: 46 is the amino acid sequence of murine CD28 (corresponding to GenBank: AAA37395.1).

SEQ ID NOs: 47 to 50 are various linkers which may be used in the bispecific polypeptides of the invention.

SEQ ID NO: 51 is the amino acid sequence of human OX40 (corresponding to GenBank: NP_003318.1) SEQ ID NOs: 52 to 88 are exemplary CDR sequences of anti-OX40 antibodies disclosed herein.

SEQ ID NOs: 89 to 124 are exemplary amino acid and nucleotide sequences of the heavy and light chain variable regions of antibodies disclosed herein.

SEQ ID NOs: 125 to 134 are exemplary amino acid and nucleotide sequences of bispecific polypeptides disclosed herein.

SEQ ID NO: 135 is an exemplary heavy chain constant region amino acid sequence.

SEQ ID NO: 136 is an exemplary light chain constant region amino acid sequence.

SEQ ID NO: 137 is an exemplary modified human heavy chain IgG4 constant region sequence with a mutation from Ser to Pro in the hinge region (position 108) and from His to Arg in the CH3 region (position 315). Mutations result in reduced serum half-life and stabilization of the core hinge of IgG4 making the IgG4 more stable, preventing Fab arm exchange.

SEQ ID NO: 138 is an exemplary wild type human heavy chain IgG4 constant region sequence. That is a sequence lacking the mutations of SEQ ID NO: 137.

SEQ ID NO: 139 is an exemplary modified human heavy chain IgG4 constant region sequence with a single mutation from Ser to Pro in the hinge region (position 108). Mutation results in stabilization of the core hinge of IgG4 making the IgG4 more stable, preventing Fab arm exchange.

SEQ ID NO: 140 is an exemplary cDNA sequence (i.e. lacking introns) encoding the IgG4 constant region of SEQ ID NO: 137.

SEQ ID NO: 141 is an exemplary genomic DNA sequence (i.e. including introns) encoding the IgG4 constant region of SEQ ID NO: 137 SEQ ID NO: 142 is an exemplary cDNA sequence (i.e. lacking introns) encoding the IgG4 constant region of SEQ ID NO: 138.

SEQ ID NO: 143 is an exemplary genomic DNA sequence (i.e. including introns) encoding the IgG4 constant region of SEQ ID NO: 138.

SEQ ID NO 144 is a linker which may be used in the bispecific polypeptides of the invention.

SEQ ID NOs: 145 and 146 are exemplary cDNA and genomic DNA sequences, respectively, encoding the IgG1 constant region of SEQ ID NO: 135.

SEQ ID NOs: 147 is an exemplary DNA sequence encoding the light chain kappa region of SEQ ID NO: 136.

SEQ ID NO: 148 is an exemplary cDNA sequence (i.e. lacking introns) encoding the IgG4 region of SEQ ID NO: 139.

SEQ ID NO: 149 is an exemplary genomic DNA sequence (i.e. including introns) encoding the IgG4 region of SEQ ID NO: 139.

Tables (Sequences)

TABLE A(1)
Exemplary heavy chain CDR
sequences (OX40 antibody)

| VH | | | | | | |
|---|---|---|---|---|---|---|
| number | SEQ | H CDR1 | SEQ | H CDR2 | SEQ | H CDR3 |
| 1166 | 52 | GFTFGGYY | 60 | ISGSGGST | 69 | ARYDYASMDY |
| 1170 | | As 1166 | 61 | IPGSGGST | 70 | ARYDYYWMDY |
| 1164 | 53 | GFTFYGSS | 62 | IYSSGGYT | 71 | ARGVPHGYFDY |
| 1168 | 54 | GFTFSGSS | 63 | ISYYGGYT | 72 | ARYFPHYYFDY |
| 1482 | 55 | GFTFSSYA | 64 | ISYYSGYT | 73 | ARGYGYLDY |
| 1490 | | As 1482 | | As 1168 | 74 | ARYYPHHYIDY |
| 1514 | 56 | GFTFGYYY | 65 | ISSYGSYT | 75 | ARSGYSNWANSFDY |
| 1520 | | As 1482 | | As 1166 | 76 | ARYYYSHGYYVY |
| | | | | | | GTLDY |
| 1524 | 57 | GFTFGSYY | 66 | IGSYYGYT | 77 | ARHDYGALDY |
| 1526 | 58 | GFTFSGYS | 67 | IGYSGYGT | 78 | ARYYFHDYAAYSLDY |
| 1542 | 59 | GFTFGSSS | 68 | IGYYSYSTS | 79 | ARGYPHHYFDY |

TABLE A(2)
Exemplary light chain CDR
sequences (OX40 antibody)

| VL | | | | | | |
|---|---|---|---|---|---|---|
| number | SEQ | L CDR1 | SEQ | L CDR2 | SEQ | L CDR3 |
| 1167 | 80 | QSISSY | 81 | AAS | 82 | QQYYWYGLST |
| 1171 | | As 1167 | | As 1167 | 83 | QQGHGSYPHT |
| 1135 | | As 1167 | | As 1167 | 84 | QQSYSTPYT |
| 1483 | | As 1167 | | As 1167 | 85 | QQYGSLLT |
| 1515 | | As 1167 | | As 1167 | 86 | QQGDYTLFT |
| 1525 | | As 1167 | | As 1167 | 87 | QQYGPSGLFT |
| 1527 | | As 1167 | | As 1167 | 88 | QQYGSDSLLT |

TABLE B
Exemplary sequences (OX40 antibody)

| SEQ | | | |
|---|---|---|---|
| ID | | | |
| NO. | CHAIN NO. | TYPE | SEQUENCE |
| 89 | 1167, light | aa | DIQMTQSPSSLSASVGDRVTITCRASQSISSYLN |
| | chain VL | | WYQQKPGKAPKLLIYAASSLQSGVPSRFSGSG |
| | | | SGTDFTLTISSLQPEDFATYYCQQYYWYGLSTF |
| | | | GQGTKLEIK |
| 90 | 1167, light | nt | GACATCCAGATGACCCAGTCTCCATCCTCCC |
| | chain VL | | TGAGCGCATCTGTAGGAGACCGCGTCACCAT |
| | | | CACTTGCCGGGCAAGTCAGAGCATTAGCAGC |
| | | | TATTTAAATTGGTATCAGCAGAAACCAGGGAA |
| | | | AGCCCCTAAGCTCCTGATCTATGCTGCATCC |
| | | | AGTTTGCAAAGTGGGGTCCCATCACGTTTCA |
| | | | GTGGCAGTGGAAGCGGGACAGATTTCACTCT |
| | | | CACCATCAGCAGTCTGCAACCTGAAGATTTTG |
| | | | CAACTTATTACTGTCAACAGTACTACTGGTAC |
| | | | GGTCTGTCCACTTTTGGCCAGGGGACCAAGC |
| | | | TGGAGATCAAA |
| 91 | 1166, heavy | aa | EVQLLESGGGLVQPGGSLRLSCAASGFTFGGY |
| | chain VH | | YMSWVRQAPGKGLEWVSAISGSGGSTYYADS |
| | | | VKGRFTISRDNSKNTLYLQMNSLRAEDTAVYYC |
| | | | ARYDYASMDYWGQGTLVTVSS |
| 92 | 1166, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTGGTGG |
| | | | TTACTACATGTCTTGGGTCCGCCAGGCTCCA |
| | | | GGGAAGGGGCTGGAGTGGGTCTCAGCTATTA |
| | | | GTGGTAGTGGTGGTAGCACATACTATGCAGA |
| | | | CTCCGTGAAGGGCCGGTTCACCATCTCCCGT |
| | | | GACAATTCCAAGAACACGCTGTATCTGCAAAT |
| | | | GAACAGCCTGCGTGCCGAGGACACGGCTGT |
| | | | ATATTATTGTGCGCGCTACGACTACGCTTCTA |
| | | | TGGACTATTGGGGCCAGGGAACCCTGGTCAC |
| | | | CGTCTCCTCA |
| 93 | 1171, light | aa | DIQMTQSPSSLSASVGDRVTITCRASQSISSYLN |
| | chain VL | | WYQQKPGKAPKLLIYAASSLQSGVPSRFSGSG |
| | | | SGTDFTLTISSLQPEDFATYYCQQGHGSYPHTF |
| | | | GQGTKLEIK |
| 94 | 1171, light | nt | GACATCCAGATGACCCAGTCTCCATCCTCCC |
| | chain VL | | TGAGCGCATCTGTAGGAGACCGCGTCACCAT |
| | | | CACTTGCCGGGCAAGTCAGAGCATTAGCAGC |
| | | | TATTTAAATTGGTATCAGCAGAAACCAGGGAA |
| | | | AGCCCCTAAGCTCCTGATCTATGCTGCATCC |
| | | | AGTTTGCAAAGTGGGGTCCCATCACGTTTCA |
| | | | GTGGCAGTGGAAGCGGGACAGATTTCACTCT |
| | | | CACCATCAGCAGTCTGCAACCTGAAGATTTTG |
| | | | CAACTTATTACTGTCAACAGGGTCATGGTTCT |
| | | | TACCCGCACACTTTTGGCCAGGGGACCAAGC |
| | | | TGGAGATCAAA |
| 95 | 1170, heavy | aa | EVQLLESGGGLVQPGGSLRLSCAASGFTFGGY |
| | chain VH | | YMSWVRQAPGKGLEWVSYIPGSGGSTYYADS |
| | | | VKGRFTISRDNSKNTLYLQMNSLRAEDTAVYYC |
| | | | ARYDYYWMDYWGQGTLVTVSS |
| 96 | 1170, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTGGTGG |
| | | | TTACTACATGTCTTGGGTCCGCCAGGCTCCA |
| | | | GGGAAGGGGCTGGAGTGGGTCTCATACATTC |
| | | | CTGGTTCTGGTGGTTCTACATACTATGCAGAC |
| | | | TCCGTGAAGGGCCGGTTCACCATCTCCCGTG |
| | | | ACAATTCCAAGAACACGCTGTATCTGCAAATG |
| | | | AACAGCCTGCGTGCCGAGGACACGGCTGTAT |
| | | | ATTATTGTGCGCGCTACGACTACTACTGGATG |
| | | | GACTATTGGGGCCAGGGAACCCTGGTCACC |
| | | | GTCTCCTCA |
| 97 | 1135, light | aa | DIQMTQSPSSLSASVGDRVTITCRASQSISSYLN |
| | chain VL | | WYQQKPGKAPKLLIYAASSLQSGVPSRFSGSG |
| | | | SGTDFTLTISSLQPEDFATYYCQQSYSTPYTFG |
| | | | QGTKLEIK |
| 98 | 1135, light | nt | GACATCCAGATGACCCAGTCTCCATCCTCCC |
| | chain VL | | TGAGCGCATCTGTAGGAGACCGCGTCACCAT |
| | | | CACTTGCCGGGCAAGTCAGAGCATTAGCAGC |
| | | | TATTTAAATTGGTATCAGCAGAAACCAGGGAA |
| | | | AGCCCCTAAGCTCCTGATCTATGCTGCATCC |
| | | | AGTTTGCAAAGTGGGGTCCCATCACGTTTCA |
| | | | GTGGCAGTGGAAGCGGGACAGATTTCACTCT |
| | | | CACCATCAGCAGTCTGCAACCTGAAGATTTTG |
| | | | CAACTTATTACTGTCAACAGAGTTACAGTACC |
| | | | CCTTATACTTTTGGCCAGGGGACCAAGCTGG |
| | | | AGATCAAA |
| 99 | 1164, heavy | aa | EVQLLESGGGLVQPGGSLRLSCAASGFTFYGS |
| | chain VH | | SMYWVRQAPGKGLEWVSGIYSSGGYTSYADS |
| | | | VKGRFTISRDNSKNTLYLQMNSLRAEDTAVYYC |
| | | | ARGVPHGYFDYWGQGTLVTVSS |
| 100 | 1164, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTTACGG |
| | | | TTCTTCTATGTACTGGGTCCGCCAGGCTCCA |
| | | | GGGAAGGGGCTGGAGTGGGTCTCAGGTATTT |
| | | | ACTCTTCTGGTGGTTACACATCTTATGCAGAC |
| | | | TCCGTGAAGGGCCGGTTCACCATCTCCCGTG |
| | | | ACAATTCCAAGAACACGCTGTATCTGCAAATG |
| | | | AACAGCCTGCGTGCCGAGGACACGGCTGTAT |
| | | | ATTATTGTGCGCGCGGTGTTCCTCATGGTTAC |
| | | | TTTGACTATTGGGGCCAGGGAACCCTGGTCA |
| | | | CCGTCTCCTCA |
| 101 | 1168, heavy | aa | EVQLLESGGGLVQPGGSLRLSCAASGFTFSGS |
| | chain VH | | SMSWVRQAPGKGLEWVSSISYYGGYTYYADS |
| | | | VKGRFTISRDNSKNTLYLQMNSLRAEDTAVYYC |
| | | | ARYFPHYYFDYWGQGTLVTVSS |
| 102 | 1168, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTAGTGG |
| | | | TTCTTCTATGTCTTGGGTCCGCCAGGCTCCA |
| | | | GGGAAGGGGCTGGAGTGGGTCTCATCTATTT |
| | | | CTTACTACGGTGGTTACACATACTATGCAGAC |
| | | | TCCGTGAAGGGCCGGTTCACCATCTCCCGTG |
| | | | ACAATTCCAAGAACACGCTGTATCTGCAAATG |
| | | | AACAGCCTGCGTGCCGAGGACACGGCTGTAT |
| | | | ATTATTGTGCGCGCTACTTCCCGCATTACTAC |
| | | | TTTGACTATTGGGGCCAGGGAACCCTGGTCA |
| | | | CCGTCTCCTCA |
| 103 | 1483, light | aa | DIQMTQSPSSLSASVGDRVTITCRASQSISSYLN |
| | chain VL | | WYQQKPGKAPKLLIYAASSLQSGVPSRFSGSG |
| | | | SGTDFTLTISSLQPEDFATYYCQQYGSLLTFGQ |
| | | | GTKLEIK |
| 104 | 1483, light | nt | GACATCCAGATGACCCAGTCTCCATCCTCCC |
| | chain VL | | TGAGCGCATCTGTAGGAGACCGCGTCACCAT |
| | | | CACTTGCCGGGCAAGTCAGAGCATTAGCAGC |
| | | | TATTTAAATTGGTATCAGCAGAAACCAGGGAA |
| | | | AGCCCCTAAGCTCCTGATCTATGCTGCATCC |
| | | | AGTTTGCAAAGTGGGGTCCCATCACGTTTCA |
| | | | GTGGCAGTGGAAGCGGGACAGATTTCACTCT |
| | | | CACCATCAGCAGTCTGCAACCTGAAGATTTTG |
| | | | CAACTTATTACTGTCAACAGTACGGTTCTCTG |
| | | | CTCACTTTTGGCCAGGGGACCAAGCTGGAGA |
| | | | TCAAA |
| 105 | 1482, heavy | aa | EVQLLESGGGLVQPGGSLRLSCAASGFTFSSY |
| | chain VH | | AMSWVRQAPGKGLEWVSYISYYSGYTYYADSV |
| | | | KGRFTISRDNSKNTLYLQMNSLRAEDTAVYYCA |
| | | | RGYGYLDYWGQGTLVTVSS |
| 106 | 1482, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTAGCAG |
| | | | CTATGCCATGAGCTGGGTCCGCCAGGCTCCA |
| | | | GGGAAGGGGCTGGAGTGGGTCTCATACATTT |
| | | | CTTACTACTCTGGTTACACATACTATGCAGAC |
| | | | TCCGTGAAGGGCCGGTTCACCATCTCCCGTG |
| | | | ACAATTCCAAGAACACGCTGTATCTGCAAATG |
| | | | AACAGCCTGCGTGCCGAGGACACGGCTGTAT |
| | | | ATTATTGTGCGCGCGGTTACGGTTACTTGGA |
| | | | CTATTGGGGCCAGGGAACCCTGGTCACCGTC |
| | | | TCCTCA |
| 107 | 1490, heavy | aa | EVQLLESGGGLVQPGGSLRLSCAASGFTFSSY |
| | chain VH | | AMSWVRQAPGKGLEWVSGISYYGGYTYYADS |
| | | | VKGRFTISRDNSKNTLYLQMNSLRAEDTAVYYC |
| | | | ARYYPHHYIDYWGQGTLVTVSS |
| 108 | 1490, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTAGCAG |
| | | | CTATGCCATGAGCTGGGTCCGCCAGGCTCCA |
| | | | GGGAAGGGGCTGGAGTGGGTCTCAGGTATTT |
| | | | CTTACTACGGTGGTTACACATACTATGCAGAC |
| | | | TCCGTGAAGGGCCGGTTCACCATCTCCCGTG |
| | | | ACAATTCCAAGAACACGCTGTATCTGCAAATG |
| | | | AACAGCCTGCGTGCCGAGGACACGGCTGTAT |
| | | | ATTATTGTGCGCGCTACTACCCGCATCATTAC |
| | | | ATTGACTATTGGGGCCAGGGAACCCTGGTCA |
| | | | CCGTCTCCTCA |
| 109 | 1515, light | aa | DIQMTQSPSSLSASVGDRVTITCRASQSISSYLN |
| | chain VL | | WYQQKPGKAPKLLIYAASSLQSGVPSRFSGSG |
| | | | SGTDFTLTISSLQPEDFATYYCQQGDYTLFTFG |
| | | | QGTKLEIK |
| 110 | 1515, light | nt | GACATCCAGATGACCCAGTCTCCATCCTCCC |
| | chain VL | | TGAGCGCATCTGTAGGAGACCGCGTCACCAT |
| | | | CACTTGCCGGGCAAGTCAGAGCATTAGCAGC |
| | | | TATTTAAATTGGTATCAGCAGAAACCAGGGAA |
| | | | AGCCCCTAAGCTCCTGATCTATGCTGCATCC |
| | | | AGTTTGCAAAGTGGGGTCCCATCACGTTTCA |
| | | | GTGGCAGTGGAAGCGGGACAGATTTCACTCT |
| | | | CACCATCAGCAGTCTGCAACCTGAAGATTTTG |
| | | | CAACTTATTACTGTCAACAGGGTGATTACACT |
| | | | CTGTTCACTTTTGGCCAGGGGACCAAGCTGG |
| | | | AGATCAAA |
| 111 | 1514, heavy | aa | EVQLLESGGGLVQPGGSLRLSCAASGFTFGYY |
| | chain VH | | YMSWVRQAPGKGLEWVSGISSYGSYTYYADS |
| | | | VKGRFTISRDNSKNTLYLQMNSLRAEDTAVYYC |
| | | | ARSGYSNWANSFDYWGQGTLVTVSS |
| 112 | 1514, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTGGTTA |
| | | | CTACTACATGTCTTGGGTCCGCCAGGCTCCA |
| | | | GGGAAGGGGCTGGAGTGGGTCTCAGGTATTT |
| | | | CTTCTTACGGTAGTTACACATACTATGCAGAC |
| | | | TCCGTGAAGGGCCGGTTCACCATCTCCCGTG |
| | | | ACAATTCCAAGAACACGCTGTATCTGCAAATG |
| | | | AACAGCCTGCGTGCCGAGGACACGGCTGTAT |
| | | | ATTATTGTGCGCGCTCTGGTTACTCTAACTGG |
| | | | GCTAACTCTTTTGACTATTGGGGCCAGGGAA |
| | | | CCCTGGTCACCGTCTCCTCA |
| 113 | 1520, heavy | aa | EVQLLESGGGLVQPGGSLRLSCAASGFTFSSY |
| | chain VH | | AMSWVRQAPGKGLEWVSAISGSGGSTYYADS |
| | | | VKGRFTISRDNSKNTLYLQMNSLRAEDTAVYYC |
| | | | ARYYYSHGYYVYGTLDYWGQGTLVTVSS |
| 114 | 1520, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTAGCAG |
| | | | CTATGCCATGAGCTGGGTCCGCCAGGCTCCA |
| | | | GGGAAGGGGCTGGAGTGGGTCTCAGCTATTA |
| | | | GTGGTAGTGGTGGTAGCACATACTATGCAGA |
| | | | CTCCGTGAAGGGCCGGTTCACCATCTCCCGT |
| | | | GACAATTCCAAGAACACGCTGTATCTGCAAAT |
| | | | GAACAGCCTGCGTGCCGAGGACACGGCTGT |
| | | | ATATTATTGTGCGCGCTACTACTACTCTCATG |
| | | | GTTACTACGTTTACGGTACTTTGGACTATTGG |
| | | | GGCCAGGGAACCCTGGTCACCGTCTCCTCA |
| 115 | 1525, light | aa | DIQMTQSPSSLSASVGDRVTITCRASQSISSYLN |
| | chain VL | | WYQQKPGKAPKLLIYAASSLQSGVPSRFSGSG |
| | | | SGTDFTLTISSLQPEDFATYYCQQYGPSGLFTF |
| | | | GQGTKLEIK |
| 116 | 1525, light | nt | GACATCCAGATGACCCAGTCTCCATCCTCCC |
| | chain VL | | TGAGCGCATCTGTAGGAGACCGCGTCACCAT |
| | | | CACTTGCCGGGCAAGTCAGAGCATTAGCAGC |
| | | | TATTTAAATTGGTATCAGCAGAAACCAGGGAA |
| | | | AGCCCCTAAGCTCCTGATCTATGCTGCATCC |
| | | | AGTTTGCAAAGTGGGGTCCCATCACGTTTCA |
| | | | GTGGCAGTGGAAGCGGGACAGATTTCACTCT |
| | | | CACCATCAGCAGTCTGCAACCTGAAGATTTTG |
| | | | CAACTTATTACTGTCAACAGTACGGTCCGTCT |
| | | | GGTCTGTTCACTTTTGGCCAGGGGACCAAGC |
| | | | TGGAGATCAAA |
| 117 | 1524, heavy | aa | EVQLLESGGGLVQPGGSLRLSCAASGFTFGSY |
| | chain VH | | YMGWVRQAPGKGLEWVSSIGSYYGYTYYADS |
| | | | VKGRFTISRDNSKNTLYLQMNSLRAEDTAVYYC |
| | | | ARHDYGALDYWGQGTLVTVSS |
| 118 | 1524, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTGGTTC |
| | | | TTACTACATGGGTTGGGTCCGCCAGGCTCCA |
| | | | GGGAAGGGGCTGGAGTGGGTCTCATCTATTG |
| | | | GTTCTTACTACGGTTACACATACTATGCAGAC |
| | | | TCCGTGAAGGGCCGGTTCACCATCTCCCGTG |
| | | | ACAATTCCAAGAACACGCTGTATCTGCAAATG |
| | | | AACAGCCTGCGTGCCGAGGACACGGCTGTAT |
| | | | ATTATTGTGCGCGCCATGACTACGGTGCTTT |
| | | | GGACTATTGGGGCCAGGGAACCCTGGTCAC |
| | | | CGTCTCCTCA |
| 119 | 1527, light | aa | DIQMTQSPSSLSASVGDRVTITCRASQSISSYLN |
| | chain VL | | WYQQKPGKAPKLLIYAASSLQSGVPSRFSGSG |
| | | | SGTDFTLTISSLQPEDFATYYCQQYGSDSLLTF |
| | | | GQGTKLEIK |
| 120 | 1527, light | nt | GACATCCAGATGACCCAGTCTCCATCCTCCC |
| | chain VL | | TGAGCGCATCTGTAGGAGACCGCGTCACCAT |
| | | | CACTTGCCGGGCAAGTCAGAGCATTAGCAGC |
| | | | TATTTAAATTGGTATCAGCAGAAACCAGGGAA |
| | | | AGCCCCTAAGCTCCTGATCTATGCTGCATCC |
| | | | AGTTTGCAAAGTGGGGTCCCATCACGTTTCA |
| | | | GTGGCAGTGGAAGCGGGACAGATTTCACTCT |
| | | | CACCATCAGCAGTCTGCAACCTGAAGATTTTG |
| | | | CAACTTATTACTGTCAACAGTACGGTTCTGAT |
| | | | TCTCTGCTCACTTTTGGCCAGGGGACCAAGC |
| | | | TGGAGATCAAA |
| 121 | 1526, heavy | aa | EVQLLESGGGLVQPGGSLRLSCAASGFTFSGY |
| | chain VH | | SMYWVRQAPGKGLEWVSGIGYSGYGTYYADS |
| | | | VKGRFTISRDNSKNTLYLQMNSLRAEDTAVYYC |
| | | | ARYYFHDYAAYSLDYWGQGTLVTVSS |
| 122 | 1526, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTTCTGG |
| | | | TTACTCTATGTACTGGGTCCGCCAGGCTCCA |
| | | | GGGAAGGGGCTGGAGTGGGTCTCAGGTATT |
| | | | GGTTACTCTGGTTACGGTACATACTATGCAGA |
| | | | CTCCGTGAAGGGCCGGTTCACCATCTCCCGT |
| | | | GACAATTCCAAGAACACGCTGTATCTGCAAAT |
| | | | GAACAGCCTGCGTGCCGAGGACACGGCTGT |
| | | | ATATTATTGTGCGCGCTACTACTTCCATGACT |
| | | | ACGCTGCTTACTCTTTGGACTATTGGGGCCA |
| | | | GGGAACCCTGGTCACCGTCTCCTCA |
| 123 | 1542, heavy | aa | EVQLLESGGGLVQPGGSLRLSCAASGFTFGSS |
| | chain VH | | SMYWVRQAPGKGLEWVSGIGYYSYSTSYADS |
| | | | VKGRFTISRDNSKNTLYLQMNSLRAEDTAVYYC |
| | | | ARGYPHHYFDYWGQGTLVTVSS |
| 124 | 1542, heavy | nt | GAGGTGCAGCTGTTGGAGAGCGGGGGAGGC |
| | chain VH | | TTGGTACAGCCTGGGGGGTCCCTGCGCCTCT |
| | | | CCTGTGCAGCCAGCGGATTCACCTTTGGTTC |
| | | | TTCTTCTATGTACTGGGTCCGCCAGGCTCCA |
| | | | GGGAAGGGGCTGGAGTGGGTCTCAGGTATT |
| | | | GGTTACTACTCTTACTCTACATCTTATGCAGA |
| | | | CTCCGTGAAGGGCCGGTTCACCATCTCCCGT |
| | | | GACAATTCCAAGAACACGCTGTATCTGCAAAT |
| | | | GAACAGCCTGCGTGCCGAGGACACGGCTGT |
| | | | ATATTATTGTGCGCGCGGTTACCCGCATCATT |
| | | | ACTTTGACTATTGGGGCCAGGGAACCCTGGT |
| | | | CACCGTCTCCTCA |

TABLE C
Exemplary variants of domain of human CD86

| SEQ | DESIG- | |
|---|---|---|
| ID NO. | NATION | SEQUENCE |
| 6 | 900 | LKIQAYFNETADLPCQFANSQNQSLSELVV |
| | | FWQDQENLVLNEVYLGKEKFDSVDSKYMGR |
| | | TSFDSDSWTLRLHNLQIKDKGIYQCVIHHK |
| | | KPSGLVKIHEMNSELSVLA |
| 7 | 901 | LKIQAYFNETADLPCQFANSQNLTLSELVV |
| | | FWQDQENLVLNEVYLGKEKFDSVHSKYMGR |
| | | TSFDSDSWTLRLHNLQIKDKGIYQCVIHHK |
| | | KPTGMIKIHEMNSELSVLT |
| 8 | 904 | LKIQAYFNETADLPCQFANSQNQSLSELIV |
| | | FWQDQENLVLNEVYLGKERFDAVDSKYMGR |
| | | TSFDSDSWTLRLHNLQIKDKGIYQCIIHHK |
| | | KPSGMVKIHQMDSELSVLA |
| 9 | 906 | LKIQAYINETADLPCQFANSQNLSLSELVV |
| | | FWQDQENLVLNEVYLGKERFDSVDSKYMGR |
| | | TSFDSDSWTLRLHNLQIKDKGFYQCIIHHK |
| | | KPTGLVKIHEMNSELSVLA |
| 10 | 907 | LKIQAYFNETADLPCQFANSQNQSLSELVV |
| | | FWQDQENLVLNEVYLGKEKFDSVHSKYMGR |
| | | TSFDSDSWTLRLHNLQIKDKGLYQCIIHHK |
| | | KPTGMIKIHEMNSELSVLA |
| 11 | 908 | LKIQAYFNETADLPCQFANSQNQSLSELVV |
| | | FWQDQENLVLNEVYLGKEKFDSVHSKYMGR |
| | | TSFDSDSWTLRLHNLQIKDKGIYQCIIHHK |
| | | KPTGMVKIHEMNSELSVLA |
| 12 | 910 | LKIQAYFNETADLPCQFANSQNQSLSELVV |
| | | FWQDQENLVLNEVYLGKEKFDSVDSKYMGR |
| | | TSFDSDSWTLRLHNLQIKDKGIYQCIIHHK |
| | | KPTGMVKIHEMNSELSVLA |
| 13 | 915 | LKIQAYFNETADLPCQFANSQNQSLSELVV |
| | | FWQDQENLILNEVYLGKEKFDSVDSKYMGR |
| | | TSFDSDSWTLRLHNLQIKDKGFYQCIIHHK |
| | | KPSGLIKIHQMDSELSVLA |
| 14 | 938 | LKIQAYFNETADLPCQFANSQNQSLSELVV |
| | | FWQDQENLILNEVYLGKEKFDSVHSKYMGR |
| | | TSFDSDSWTLRLHNLQIKDKGIYQCIIHHK |
| | | KPTGMVKIHQMNSELSVLA |
| 15 | 1038 | APLKIQAYFNETADLPCQFANSQNLSLSEL |
| | | VVFWQDQENLVLNEVYLGKEKFDSVDSKYM |
| | | GRTSFDSDSWTLRLHNLQIKDKGIYQCIIH |
| | | HKKPTGMVKIHEMNSELSVLA |
| 16 | 1039 | APLKIQAYFNETADLPCQFANSQNLSLSEL |
| | | VVFWQDQENLVLNEVYLGKEKFDSVSSKYM |
| | | GRTSFDSDSWTLRLHNLQIKDKGIYQCIIH |
| | | HKKPSGMVKIHQMDSELSVLA |
| 17 | 1040 | APLKIQAYFNETADLPCQFANSQNLSLSEL |
| | | VVFWQDQENLVLNEVYLGKERFDSVDSKYM |
| | | GRTSFDSDSWTLRLHNLQIKDKGRYQCIIH |
| | | HKKPTGMINIHQMNSELSVLA |
| 18 | 1041 | APLKIQAYLNETADLPCQFANSQNLSLSEL |
| | | VVFWQDQENLVLNEVYLGKEKFDSVDSKYM |
| | | GRTSFDSDSWTLRLHNLQIKDKGIYQCIIH |
| | | HKKPTGLVKIHEMNSELSVLA |
| 19 | 1042 | APLKIQAYFNETADLPCQFANSQNLSLSEL |
| | | VVFWQDQENLVLNEVYLGKEIFDSVSSKYM |
| | | GRTSFDSDSWTLRLHNLQIKDKGIYQCIIH |
| | | HKKPSGMVKIHQMDSELSVLA |
| 20 | 1043 | APLKIQAYFNETADLPCQFANSQNLSLSEL |
| | | VVFWQDQENLVLNEVYLGKEKFDSVDSKYM |
| | | GRTSFDSDSWTLRLHNLQIKDKGIYQCIIH |
| | | HKKPTGMIKIHEMNSELSVLA |
| 21 | 1044 | APLKIQAYFNETADLPCQFANSQNLTLSEL |
| | | VVFWQDQENLVLNEVYLGKEKFDSVSSKYM |
| | | GRTSFDSDSWTLRLHNLQIKDKGIYQCIIH |
| | | HKKPTGMIKIHEMSSELSVLA |
| 22 | 1045 | APLKIQAYFNETADLPCQFANSQNLTLSEL |
| | | VVFWQDQENLVLNEVYLGKEKFDSVDSKYM |
| | | GRTSFDSDSWTLRLHNLQIKDKGLYQCIIH |
| | | HKKPTGLVKIHEMNSELSVLA |
| 23 | 1046 | APLKIQAYFNETADLPCQFANSQNQSLSEL |
| | | VVFWQDQENLVLNEVYLGKEKFDSVDSKYM |
| | | GRTSFDSDSWTLRLHNLQIEDKGIYQCIIH |
| | | HKKPSGMVKIHQMDSELSVLA |
| 24 | 1047 | APLKIQAYFNETADLPCQFANSQNLSLSEL |
| | | VVFWQDQENLVLNEVYLGKEKFDSVDSKYM |
| | | GRTSFDSDSWTLRLHNLQIKDKGIYQCIIH |
| | | HKKPTGLVKIHEMNSELSVLA |

TABLE D
Exemplary polypeptides for OX40 and CTLA-4

| SEQ | | | |
|---|---|---|---|
| ID | DESIG- | | |
| NO. | NATION | TYPE | SEQUENCE |
| 125 | 1261 = 1167 | aa | DIQMTQSPSSLSASVGDRVTITCRASQSISSYLN |
| | light chain | | WYQQKPGKAPKLLIYAASSLQSGVPSRFSGSG |
| | VL, with | | SGTDFTLTISSLQPEDFATYYCQQYYWYGLSTF |
| | constant kappa | | GQGTKLEIKRTVAAPSVFIFPPSDEQLKSGTASV |
| | sequence, linker | | VCLLNNFYPREAKVQWKVDNALQSGNSQESVT |
| | (underlined) and | | EQDSKDSTYSLSSTLTLSKADYEKHKVYACEVT |
| | CD86 mutant | | HQGLSSPVTKSFNRGECSGGGGSGGGGSAPL |
| | 1040 | | KIQAYFNETADLPCQFANSQNLSLSELVVFWQD |
| | | | QENLVLNEVYLGKERFDSVDSKYMGRTSFDSD |
| | | | SWTLRLHNLQIKDKGRYQCIIHHKKPTGMINIHQ |
| | | | MNSELSVLA |
| | | | NB. LIGHT CHAIN PREFERABLY ASSEMBLES |
| | | | WITH A HEAVY CHAIN COMPRISING THE 1166 |
| | | | VH SEQUENCE |
| | | | THUS, COMPLETE MOLECULE MAY BE |
| | | | DESIGNATED 1166/1261 |
| 126 | 1261 = 1267 | nt | GACATCCAGATGACCCAGTCTCCATCCTCCC |
| | light chain VL, | | TGAGCGCATCTGTAGGAGACCGCGTCACCAT |
| | with constant | | CACTTGCCGGGCAAGTCAGAGCATTAGCAGC |
| | kappa | | TATTTAAATTGGTATCAGCAGAAACCAGGGAA |
| | sequence, linker | | AGCCCCTAAGCTCCTGATCTATGCTGCATCC |
| | and CD86 | | AGTTTGCAAAGTGGGGTCCCATCACGTTTCA |
| | mutant 1040 | | GTGGCAGTGGAAGCGGGACAGATTTCACTCT |
| | | | CACCATCAGCAGTCTGCAACCTGAAGATTTTG |
| | | | CAACTTATTACTGTCAACAGTACTACTGGTAC |
| | | | GGTCTGTCCACTTTTGGCCAGGGGACCAAGC |
| | | | TGGAGATCAAACgtgagtcgtacgctagcaagcttgatatc |
| | | | gaattctaaactctgagggggtcggatgacgtggccattctttgcct |
| | | | aaagcattgagtttactgcaaggtcagaaaagcatgcaaagccct |
| | | | cagaatggctgcaaagagctccaacaaaacaatttagaactttatt |
| | | | aaggaatagggggaagctaggaagaaactcaaaacatcaaga |
| | | | ttttaaatacgcttcttggtctccttgctataattatctgggataagcatg |
| | | | ctgttttctgtctgtccctaacatgccctgtgattatccgcaaacaaca |
| | | | cacccaagggcagaactttgttacttaaacaccatcctgtttgcttctt |
| | | | tcctcagGAACTGTGGCTGCACCATCTGTCTTCA |
| | | | TCTTCCCGCCATCTGATGAGCAGTTGAAATCT |
| | | | GGAACTGCCTCTGTTGTGTGCCTGCTGAATA |
| | | | ACTTCTATCCCAGAGAGGCCAAAGTACAGTG |
| | | | GAAGGTGGATAACGCCCTCCAATCGGGTAAC |
| | | | TCCCAGGAGAGTGTCACAGAGCAGGACAGCA |
| | | | AGGACAGCACCTACAGCCTCAGCAGCACCCT |
| | | | GACGCTGAGCAAAGCAGACTACGAGAAACAC |
| | | | AAAGTCTACGCCTGCGAAGTCACCCATCAGG |
| | | | GCCTGAGCTCGCCCGTCACAAAGAGCTTCAA |
| | | | CAGGGGAGAGTGTAGCGGAGGAGGAGGAAG |
| | | | CGGAGGAGGAGGAAGCGCCCCCCTCAAAAT |
| | | | CCAAGCGTACTTCAACGAAACTGCAGACTTA |
| | | | CCGTGTCAGTTTGCCAATTCGCAGAATCTGA |
| | | | GCCTGAGCGAACTGGTGGTTTTCTGGCAGGA |
| | | | TCAGGAGAACCTGGTTCTGAACGAAGTCTAT |
| | | | CTGGGCAAAGAGCGGTTCGACAGCGTGGAC |
| | | | AGCAAGTATATGGGCCGCACCAGCTTTGATA |
| | | | GCGACAGCTGGACCCTGCGTCTGCACAATCT |
| | | | GCAAATCAAAGATAAGGGTAGGTACCAGTGC |
| | | | ATTATCCACCATAAGAAGCCGACGGGTATGA |
| | | | TTAATATTCACCAAATGAACTCCGAGTTGTCT |
| | | | GTCCTGGCG |
| 127 | 1263 = 1171 | aa | DIQMTQSPSSLSASVGDRVTITCRASQSISSYLN |
| | light chain VL, | | WYQQKPGKAPKLLIYAASSLQSGVPSRFSGSG |
| | with constant | | SGTDFTLTISSLQPEDFATYYCQQGHGSYPHTF |
| | kappa | | GQGTKLEIKRTVAAPSVFIFPPSDEQLKSGTASV |
| | sequence, linker | | VCLLNNFYPREAKVQWKVDNALQSGNSQESVT |
| | (underlined) and | | EQDSKDSTYSLSSTLTLSKADYEKHKVYACEVT |
| | CD86 mutant | | HQGLSSPVTKSFNRGECSGGGGSGGGGSAPL |
| | 1040 | | KIQAYFNETADLPCQFANSQNLSLSELVVFWQD |
| | | | QENLVLNEVYLGKERFDSVDSKYMGRTSFDSD |
| | | | SWTLRLHNLQIKDKGRYQCIIHHKKPTGMINIHQ |
| | | | MNSELSVLA |
| | | | LIGHT CHAIN PREFERABLY ASSEMBLES |
| | | | WITH A HEAVY CHAIN COMPRISING THE 1170 |
| | | | VH SEQUENCE |
| | | | THUS, COMPLETE MOLECULE MAY BE |
| | | | DESIGNATED 1170/1263 |
| 128 | 1263 = 1171 | nt | GACATCCAGATGACCCAGTCTCCATCCTCCC |
| | light chain VL, | | TGAGCGCATCTGTAGGAGACCGCGTCACCAT |
| | with constant | | CACTTGCCGGGCAAGTCAGAGCATTAGCAGC |
| | kappa | | TATTTAAATTGGTATCAGCAGAAACCAGGGAA |
| | sequence, linker | | AGCCCCTAAGCTCCTGATCTATGCTGCATCC |
| | and CD86 | | AGTTTGCAAAGTGGGGTCCCATCACGTTTCA |
| | mutant 1040 | | GTGGCAGTGGAAGCGGGACAGATTTCACTCT |
| | | | CACCATCAGCAGTCTGCAACCTGAAGATTTTG |
| | | | CAACTTATTACTGTCAACAGGGTCATGGTTCT |
| | | | TACCCGCACACTTTTGGCCAGGGGACCAAGC |
| | | | TGGAGATCAAACgtgagtcgtacgctagcaagcttgatatc |
| | | | gaattctaaactctgagggggtcggatgacgtggccattctttgcct |
| | | | aaagcattgagtttactgcaaggtcagaaaagcatgcaaagccct |
| | | | cagaatggctgcaaagagctccaacaaaacaatttagaactttatt |
| | | | aaggaatagggggaagctaggaagaaactcaaaacatcaaga |
| | | | ttttaaatacgcttcttggtctccttgctataattatctgggataagcatg |
| | | | ctgttttctgtctgtccctaacatgccctgtgattatccgcaaacaaca |
| | | | cacccaagggcagaactttgttacttaaacaccatcctgtttgcttctt |
| | | | tcctcagGAACTGTGGCTGCACCATCTGTCTTCA |
| | | | TCTTCCCGCCATCTGATGAGCAGTTGAAATCT |
| | | | GGAACTGCCTCTGTTGTGTGCCTGCTGAATA |
| | | | ACTTCTATCCCAGAGAGGCCAAAGTACAGTG |
| | | | GAAGGTGGATAACGCCCTCCAATCGGGTAAC |
| | | | TCCCAGGAGAGTGTCACAGAGCAGGACAGCA |
| | | | AGGACAGCACCTACAGCCTCAGCAGCACCCT |
| | | | GACGCTGAGCAAAGCAGACTACGAGAAACAC |
| | | | AAAGTCTACGCCTGCGAAGTCACCCATCAGG |
| | | | GCCTGAGCTCGCCCGTCACAAAGAGCTTCAA |
| | | | CAGGGGAGAGTGTAGCGGAGGAGGAGGAAG |
| | | | CGGAGGAGGAGGAAGCGCCCCCCTCAAAAT |
| | | | CCAAGCGTACTTCAACGAAACTGCAGACTTA |
| | | | CCGTGTCAGTTTGCCAATTCGCAGAATCTGA |
| | | | GCCTGAGCGAACTGGTGGTTTTCTGGCAGGA |
| | | | TCAGGAGAACCTGGTTCTGAACGAAGTCTAT |
| | | | CTGGGCAAAGAGCGGTTCGACAGCGTGGAC |
| | | | AGCAAGTATATGGGCCGCACCAGCTTTGATA |
| | | | GCGACAGCTGGACCCTGCGTCTGCACAATCT |
| | | | GCAAATCAAAGATAAGGGTAGGTACCAGTGC |
| | | | ATTATCCACCATAAGAAGCCGACGGGTATGA |
| | | | TTAATATTCACCAAATGAACTCCGAGTTGTCT |
| | | | GTCCTGGCG |
| 129 | 1141 = 1135 | aa | DIQMTQSPSSLSASVGDRVTITCRASQSISSYLN |
| | light chain VL, | | WYQQKPGKAPKLLIYAASSLQSGVPSRFSGSG |
| | with constant | | SGTDFTLTISSLQPEDFATYYCQQSYSTPYTFG |
| | kappa | | QGTKLEIKRTVAAPSVFIFPPSDEQLKSGTASVV |
| | sequence, linker | | CLLNNFYPREAKVQWKVDNALQSGNSQESVTE |
| | (underlined) and | | QDSKDSTYSLSSTLTLSKADYEKHKVYACEVTH |
| | CD86 mutant | | QGLSSPVTKSFNRGECSGGGGSGGGGSAPLKI |
| | 1040 | | QAYFNETADLPCQFANSQNLSLSELVVFWQDQ |
| | | | ENLVLNEVYLGKERFDSVDSKYMGRTSFDSDS |
| | | | WTLRLHNLQIKDKGRYQCIIHHKKPTGMINIHQM |
| | | | NSELSVLA |
| | | | LIGHT CHAIN PREFERABLY ASSEMBLES |
| | | | WITH A HEAVY CHAIN COMPRISING ANY ONE |
| | | | OF THE 1164, 1168, 1520, OR 1542 VH |
| | | | SEQUENCES |
| | | | THUS, COMPLETE MOLECULES MAY BE |
| | | | DESIGNATED 1164/1141, 1168/1141, 1520/1141 |
| | | | OR 1542/1141 |
| 130 | 1141 = 1135 | nt | GACATCCAGATGACCCAGTCTCCATCCTCCC |
| | light chain VL, | | TGAGCGCATCTGTAGGAGACCGCGTCACCAT |
| | with constant | | CACTTGCCGGGCAAGTCAGAGCATTAGCAGC |
| | kappa | | TATTTAAATTGGTATCAGCAGAAACCAGGGAA |
| | sequence, linker | | AGCCCCTAAGCTCCTGATCTATGCTGCATCC |
| | and CD86 | | AGTTTGCAAAGTGGGGTCCCATCACGTTTCA |
| | mutant 1040 | | GTGGCAGTGGAAGCGGGACAGATTTCACTCT |
| | | | CACCATCAGCAGTCTGCAACCTGAAGATTTTG |
| | | | CAACTTATTACTGTCAACAGAGTTACAGTACC |
| | | | CCTTATACTTTTGGCCAGGGGACCAAGCTGG |
| | | | AGATCAAACgtgagtcgtacgctagcaagcttgatatcgaatt |
| | | | ctaaactctgagggggtcggatgacgtggccattctttgcctaaag |
| | | | cattgagtttactgcaaggtcagaaaagcatgcaaagccctcaga |
| | | | atggctgcaaagagctccaacaaaacaatttagaactttattaagg |
| | | | aatagggggaagctaggaagaaactcaaaacatcaagattttaa |
| | | | atacgcttcttggtctccttgctataattatctgggataagcatgctgttt |
| | | | tctgtctgtccctaacatgccctgtgattatccgcaaacaacacacc |
| | | | caagggcagaactttgttacttaaacaccatcctgtttgcttctttcctc |
| | | | agGAACTGTGGCTGCACCATCTGTCTTCATCT |
| | | | TCCCGCCATCTGATGAGCAGTTGAAATCTGG |
| | | | AACTGCCTCTGTTGTGTGCCTGCTGAATAACT |
| | | | TCTATCCCAGAGAGGCCAAAGTACAGTGGAA |
| | | | GGTGGATAACGCCCTCCAATCGGGTAACTCC |
| | | | CAGGAGAGTGTCACAGAGCAGGACAGCAAG |
| | | | GACAGCACCTACAGCCTCAGCAGCACCCTGA |
| | | | CGCTGAGCAAAGCAGACTACGAGAAACACAA |
| | | | AGTCTACGCCTGCGAAGTCACCCATCAGGGC |
| | | | CTGAGCTCGCCCGTCACAAAGAGCTTCAACA |
| | | | GGGGAGAGTGTAGCGGAGGAGGAGGAAGCG |
| | | | GAGGAGGAGGAAGCGCCCCCCTCAAAATCC |
| | | | AAGCGTACTTCAACGAAACTGCAGACTTACC |
| | | | GTGTCAGTTTGCCAATTCGCAGAATCTGAGC |
| | | | CTGAGCGAACTGGTGGTTTTCTGGCAGGATC |
| | | | AGGAGAACCTGGTTCTGAACGAAGTCTATCT |
| | | | GGGCAAAGAGCGGTTCGACAGCGTGGACAG |
| | | | CAAGTATATGGGCCGCACCAGCTTTGATAGC |
| | | | GACAGCTGGACCCTGCGTCTGCACAATCTGC |
| | | | AAATCAAAGATAAGGGTAGGTACCAGTGCATT |
| | | | ATCCACCATAAGAAGCCGACGGGTATGATTA |
| | | | ATATTCACCAAATGAACTCCGAGTTGTCTGTC |
| | | | CTGGCG |
| 131 | 1581 = 1515 | aa | DIQMTQSPSSLSASVGDRVTITCRASQSISSYLN |
| | light chain VL, | | WYQQKPGKAPKLLIYAASSLQSGVPSRFSGSG |
| | with constant | | SGTDFTLTISSLQPEDFATYYCQQGDYTLFTFG |
| | kappa | | QGTKLEIKRTVAAPSVFIFPPSDEQLKSGTASVV |
| | sequence, linker | | CLLNNFYPREAKVQWKVDNALQSGNSQESVTE |
| | (underlined) and | | QDSKDSTYSLSSTLTLSKADYEKHKVYACEVTH |
| | CD86 mutant | | QGLSSPVTKSFNRGECSGGGGSGGGGSAPLKI |
| | 1040 | | QAYFNETADLPCQFANSQNLSLSELVVFWQDQ |
| | | | ENLVLNEVYLGKERFDSVDSKYMGRTSFDSDS |
| | | | WTLRLHNLQIKDKGRYQCIIHHKKPTGMINIHQM |
| | | | NSELSVLA |
| | | | LIGHT CHAIN PREFERABLY ASSEMBLES |
| | | | WITH A HEAVY CHAIN COMPRISING THE 1514 |
| | | | VH SEQUENCE |
| | | | THUS, COMPLETE MOLECULE MAY BE |
| | | | DESIGNATED 1514/1581 |
| 132 | 1581 = 1515 | nt | GACATCCAGATGACCCAGTCTCCATCCTCCC |
| | light chain VL, | | TGAGCGCATCTGTAGGAGACCGCGTCACCAT |
| | with constant | | CACTTGCCGGGCAAGTCAGAGCATTAGCAGC |
| | kappa | | TATTTAAATTGGTATCAGCAGAAACCAGGGAA |
| | sequence, linker | | AGCCCCTAAGCTCCTGATCTATGCTGCATCC |
| | and CD86 | | AGTTTGCAAAGTGGGGTCCCATCACGTTTCA |
| | mutant 1040 | | GTGGCAGTGGAAGCGGGACAGATTTCACTCT |
| | | | CACCATCAGCAGTCTGCAACCTGAAGATTTTG |
| | | | CAACTTATTACTGTCAACAGGGTGATTACACT |
| | | | CTGTTCACTTTTGGCCAGGGGACCAAGCTGG |
| | | | AGATCAAACgtgagtcgtacgctagcaagcttgatatcgaatt |
| | | | ctaaactctgagggggtcggatgacgtggccattctttgcctaaag |
| | | | cattgagtttactgcaaggtcagaaaagcatgcaaagccctcaga |
| | | | atggctgcaaagagctccaacaaaacaatttagaactttattaagg |
| | | | aatagggggaagctaggaagaaactcaaaacatcaagattttaa |
| | | | atacgcttcttggtctccttgctataattatctgggataagcatgctgttt |
| | | | tctgtctgtccctaacatgccctgtgattatccgcaaacaacacacc |
| | | | caagggcagaactttgttacttaaacaccatcctgtttgcttctttcctc |
| | | | agGAACTGTGGCTGCACCATCTGTCTTCATCT |
| | | | TCCCGCCATCTGATGAGCAGTTGAAATCTGG |
| | | | AACTGCCTCTGTTGTGTGCCTGCTGAATAACT |
| | | | TCTATCCCAGAGAGGCCAAAGTACAGTGGAA |
| | | | GGTGGATAACGCCCTCCAATCGGGTAACTCC |
| | | | CAGGAGAGTGTCACAGAGCAGGACAGCAAG |
| | | | GACAGCACCTACAGCCTCAGCAGCACCCTGA |
| | | | CGCTGAGCAAAGCAGACTACGAGAAACACAA |
| | | | AGTCTACGCCTGCGAAGTCACCCATCAGGGC |
| | | | CTGAGCTCGCCCGTCACAAAGAGCTTCAACA |
| | | | GGGGAGAGTGTAGCGGAGGAGGAGGAAGCG |
| | | | GAGGAGGAGGAAGCGCCCCCCTCAAAATCC |
| | | | AAGCGTACTTCAACGAAACTGCAGACTTACC |
| | | | GTGTCAGTTTGCCAATTCGCAGAATCTGAGC |
| | | | CTGAGCGAACTGGTGGTTTTCTGGCAGGATC |
| | | | AGGAGAACCTGGTTCTGAACGAAGTCTATCT |
| | | | GGGCAAAGAGCGGTTCGACAGCGTGGACAG |
| | | | CAAGTATATGGGCCGCACCAGCTTTGATAGC |
| | | | GACAGCTGGACCCTGCGTCTGCACAATCTGC |
| | | | AAATCAAAGATAAGGGTAGGTACCAGTGCATT |
| | | | ATCCACCATAAGAAGCCGACGGGTATGATTA |
| | | | ATATTCACCAAATGAACTCCGAGTTGTCTGTC |
| | | | CTGGCG |
| 133 | 1585 = 1527 | aa | DIQMTQSPSSLSASVGDRVTITCRASQSISSYLN |
| | light chain VL, | | WYQQKPGKAPKLLIYAASSLQSGVPSRFSGSG |
| | with constant | | SGTDFTLTISSLQPEDFATYYCQQYGSDSLLTF |
| | kappa | | GQGTKLEIKRTVAAPSVFIFPPSDEQLKSGTASV |
| | sequence, linker | | VCLLNNFYPREAKVQWKVDNALQSGNSQESVT |
| | (underlined) and | | EQDSKDSTYSLSSTLTLSKADYEKHKVYACEVT |
| | CD86 mutant | | HQGLSSPVTKSFNRGECSGGGGSGGGGSAPL |
| | 1040 | | KIQAYFNETADLPCQFANSQNLSLSELVVFWQD |
| | | | QENLVLNEVYLGKERFDSVDSKYMGRTSFDSD |
| | | | SWTLRLHNLQIKDKGRYQCIIHHKKPTGMINIHQ |
| | | | MNSELSVLA |
| | | | LIGHT CHAIN PREFERABLY ASSEMBLES |
| | | | WITH A HEAVY CHAIN COMPRISING THE 1526 |
| | | | VH SEQUENCE |
| | | | THUS, COMPLETE MOLECULE MAY BE |
| | | | DESIGNATED 1526/1585 |
| 134 | 1585 = 1527 | nt | GACATCCAGATGACCCAGTCTCCATCCTCCC |
| | light chain VL, | | TGAGCGCATCTGTAGGAGACCGCGTCACCAT |
| | with constant | | CACTTGCCGGGCAAGTCAGAGCATTAGCAGC |
| | kappa | | TATTTAAATTGGTATCAGCAGAAACCAGGGAA |
| | sequence, linker | | AGCCCCTAAGCTCCTGATCTATGCTGCATCC |
| | and CD86 | | AGTTTGCAAAGTGGGGTCCCATCACGTTTCA |
| | mutant 1040 | | GTGGCAGTGGAAGCGGGACAGATTTCACTCT |
| | | | CACCATCAGCAGTCTGCAACCTGAAGATTTTG |
| | | | CAACTTATTACTGTCAACAGTACGGTTCTGAT |
| | | | TCTCTGCTCACTTTTGGCCAGGGGACCAAGC |
| | | | TGGAGATCAAACgtgagtcgtacgctagcaagcttgatatc |
| | | | gaattctaaactctgagggggtcggatgacgtggccattctttgcct |
| | | | aaagcattgagtttactgcaaggtcagaaaagcatgcaaagccct |
| | | | cagaatggctgcaaagagctccaacaaaacaatttagaactttatt |
| | | | aaggaatagggggaagctaggaagaaactcaaaacatcaaga |
| | | | ttttaaatacgcttcttggtctccttgctataattatctgggataagcatg |
| | | | ctgttttctgtctgtccctaacatgccctgtgattatccgcaaacaaca |
| | | | cacccaagggcagaactttgttacttaaacaccatcctgtttgcttctt |
| | | | tcctcagGAACTGTGGCTGCACCATCTGTCTTCA |
| | | | TCTTCCCGCCATCTGATGAGCAGTTGAAATCT |
| | | | GGAACTGCCTCTGTTGTGTGCCTGCTGAATA |
| | | | ACTTCTATCCCAGAGAGGCCAAAGTACAGTG |
| | | | GAAGGTGGATAACGCCCTCCAATCGGGTAAC |
| | | | TCCCAGGAGAGTGTCACAGAGCAGGACAGCA |
| | | | AGGACAGCACCTACAGCCTCAGCAGCACCCT |
| | | | GACGCTGAGCAAAGCAGACTACGAGAAACAC |
| | | | AAAGTCTACGCCTGCGAAGTCACCCATCAGG |
| | | | GCCTGAGCTCGCCCGTCACAAAGAGCTTCAA |
| | | | CAGGGGAGAGTGTAGCGGAGGAGGAGGAAG |
| | | | CGGAGGAGGAGGAAGCGCCCCCCTCAAAAT |
| | | | CCAAGCGTACTTCAACGAAACTGCAGACTTA |
| | | | CCGTGTCAGTTTGCCAATTCGCAGAATCTGA |
| | | | GCCTGAGCGAACTGGTGGTTTTCTGGCAGGA |
| | | | TCAGGAGAACCTGGTTCTGAACGAAGTCTAT |
| | | | CTGGGCAAAGAGCGGTTCGACAGCGTGGAC |
| | | | AGCAAGTATATGGGCCGCACCAGCTTTGATA |
| | | | GCGACAGCTGGACCCTGCGTCTGCACAATCT |
| | | | GCAAATCAAAGATAAGGGTAGGTACCAGTGC |
| | | | ATTATCCACCATAAGAAGCCGACGGGTATGA |
| | | | TTAATATTCACCAAATGAACTCCGAGTTGTCT |
| | | | GTCCTGGCG |

TABLE E
Exemplary polynucleotides encoding B2-CTLA-4

| SEQ | | |
|---|---|---|
| ID | | |
| 25 | 900 | CTCAAAATCCAAGCGTACTTCAACGAAACTGCAGACT |
| | | TACCGTGTCAGTTTGCCAATTCGCAGAATCAAAGCCT |
| | | GAGCGAACTGGTGGTTTTCTGGCAGGATCAGGAGAAC |
| | | CTGGTTCTGAACGAAGTCTATCTGGGCAAAGAGAAAT |
| | | TCGACAGCGTGGACAGCAAGTATATGGGCCGCACCAG |
| | | CTTTGATAGCGACAGCTGGACCCTGCGTCTGCACAAT |
| | | CTGCAAATCAAAGATAAGGGTATCTACCAGTGCGTGA |
| | | TCCACCATAAGAAGCCGAGCGGTCTGGTGAAGATTCA |
| | | CGAGATGAACTCCGAGTTGTCTGTCCTGGCG |
| 26 | 901 | CTCAAAATCCAAGCGTACTTCAACGAAACTGCAGACT |
| | | TACCGTGTCAGTTTGCCAATTCGCAGAATCTGACCCT |
| | | GAGCGAACTGGTGGTTTTCTGGCAGGATCAGGAGAAC |
| | | CTGGTTCTGAACGAAGTCTATCTGGGCAAAGAGAAAT |
| | | TCGACAGCGTGCATAGCAAGTATATGGGCCGCACCAG |
| | | CTTTGATAGCGACAGCTGGACCCTGCGTCTGCACAAT |
| | | CTGCAAATCAAAGATAAGGGTATCTACCAGTGCGTGA |
| | | TCCACCATAAGAAGCCGACGGGTATGATTAAGATTCA |
| | | CGAGATGAACTCCGAGTTGTCTGTCCTGACC |
| 27 | 904 | CTCAAAATCCAAGCGTACTTCAACGAAACTGCAGACT |
| | | TACCGTGTCAGTTTGCCAATTCGCAGAATCAAAGCCT |
| | | GAGCGAACTGATCGTTTTCTGGCAGGATCAGGAGAAC |
| | | CTGGTTCTGAACGAAGTCTATCTGGGCAAAGAGCGGT |
| | | TCGACGCCGTGGACAGCAAGTATATGGGCCGCACCAG |
| | | CTTTGATAGCGACAGCTGGACCCTGCGTCTGCACAAT |
| | | CTGCAAATCAAAGATAAGGGTATCTACCAGTGCATTA |
| | | TCCACCATAAGAAGCCGAGCGGTATGGTGAAGATTCA |
| | | CCAAATGGACTCCGAGTTGTCTGTCCTGGCG |
| 28 | 906 | CTCAAAATCCAAGCGTACATCAACGAAACTGCAGACT |
| | | TACCGTGTCAGTTTGCCAATTCGCAGAATCTGAGCCT |
| | | GAGCGAACTGGTGGTTTTCTGGCAGGATCAGGAGAAC |
| | | CTGGTTCTGAACGAAGTCTATCTGGGCAAAGAGCGGT |
| | | TCGACAGCGTGGACAGCAAGTATATGGGCCGCACCAG |
| | | CTTTGATAGCGACAGCTGGACCCTGCGTCTGCACAAT |
| | | CTGCAAATCAAAGATAAGGGTTTCTACCAGTGCATTA |
| | | TCCACCATAAGAAGCCGACGGGTCTGGTGAAGATTCA |
| | | CGAGATGAACTCCGAGTTGTCTGTCCTGGCG |
| 29 | 907 | CTCAAAATCCAAGCGTACTTCAACGAAACTGCAGACT |
| | | TACCGTGTCAGTTTGCCAATTCGCAGAATCAAAGCCT |
| | | GAGCGAACTGGTGGTTTTCTGGCAGGATCAGGAGAAC |
| | | CTGGTTCTGAACGAAGTCTATCTGGGCAAAGAGAAAT |
| | | TCGACAGCGTGCATAGCAAGTATATGGGCCGCACCAG |
| | | CTTTGATAGCGACAGCTGGACCCTGCGTCTGCACAAT |
| | | CTGCAAATCAAAGATAAGGGTCTGTACCAGTGCATTA |
| | | TCCACCATAAGAAGCCGACGGGTATGATTAAGATTCA |
| | | CGAGATGAACTCCGAGTTGTCTGTCCTGGCG |
| 30 | 908 | CTCAAAATCCAAGCGTACTTCAACGAAACTGCAGACT |
| | | TACCGTGTCAGTTTGCCAATTCGCAGAATCAAAGCCT |
| | | GAGCGAACTGGTGGTTTTCTGGCAGGATCAGGAGAAC |
| | | CTGGTTCTGAACGAAGTCTATCTGGGCAAAGAGAAAT |
| | | TCGACAGCGTGCATAGCAAGTATATGGGCCGCACCAG |
| | | CTTTGATAGCGACAGCTGGACCCTGCGTCTGCACAAT |
| | | CTGCAAATCAAAGATAAGGGTATCTACCAGTGCATTA |
| | | TCCACCATAAGAAGCCGACGGGTATGGTGAAGATTCA |
| | | CGAGATGAACTCCGAGTTGTCTGTCCTGGCG |
| 31 | 910 | CTCAAAATCCAAGCGTACTTCAACGAAACTGCAGACT |
| | | TACCGTGTCAGTTTGCCAATTCGCAGAATCAAAGCCT |
| | | GAGCGAACTGGTGGTTTTCTGGCAGGATCAGGAGAAC |
| | | CTGGTTCTGAACGAAGTCTATCTGGGCAAAGAGAAAT |
| | | TCGACAGCGTGGACAGCAAGTATATGGGCCGCACCAG |
| | | CTTTGATAGCGACAGCTGGACCCTGCGTCTGCACAAT |
| | | CTGCAAATCAAAGATAAGGGTATCTACCAGTGCATTA |
| | | TCCACCATAAGAAGCCGACGGGTATGGTGAAGATTCA |
| | | CGAGATGAACTCCGAGTTGTCTGTCCTGGCG |
| 32 | 915 | CTCAAAATCCAAGCGTACTTCAACGAAACTGCAGACT |
| | | TACCGTGTCAGTTTGCCAATTCGCAGAATCAAAGCCT |
| | | GAGCGAACTGGTGGTTTTCTGGCAGGATCAGGAGAAC |
| | | CTGATCCTGAACGAAGTCTATCTGGGCAAAGAGAAAT |
| | | TCGACAGCGTGGACAGCAAGTATATGGGCCGCACCAG |
| | | CTTTGATAGCGACAGCTGGACCCTGCGTCTGCACAAT |
| | | CTGCAAATCAAAGATAAGGGTTTCTACCAGTGCATTA |
| | | TCCACCATAAGAAGCCGAGCGGTCTGATTAAGATTCA |
| | | CCAAATGGACTCCGAGTTGTCTGTCCTGGCG |
| 33 | 938 | CTCAAAATCCAAGCGTACTTCAACGAAACTGCAGACT |
| | | TACCGTGTCAGTTTGCCAATTCGCAGAATCTGAGCCT |
| | | GAGCGAACTGGTGGTTTTCTGGCAGGATCAGGAGAAC |
| | | CTGATCCTGAACGAAGTCTATCTGGGCAAAGAGCGGT |
| | | TCGACAGCGTGCATAGCAAGTATATGGGCCGCACCAG |
| | | CTTTGATAGCGACAGCTGGACCCTGCGTCTGCACAAT |
| | | CTGCAAATCAAAGATAAGGGTCTGTACCAGTGCATTA |
| | | TCCACCATAAGAAGCCGAGCGGTATGGTGAAGATTCA |
| | | CGAGATGAACTCCGAGTTGTCTGTCCTGGCG |
| 34 | 1038 | GCCCCCCTCAAAATCCAAGCGTACTTCAACGAAACTG |
| | | CAGACTTACCGTGTCAGTTTGCCAATTCGCAGAATCT |
| | | GAGCCTGAGCGAACTGGTGGTTTTCTGGCAGGATCAG |
| | | GAGAACCTGGTTCTGAACGAAGTCTATCTGGGCAAAG |
| | | AGAAATTCGACAGCGTGGACAGCAAGTATATGGGCCG |
| | | CACCAGCTTTGATAGCGACAGCTGGACCCTGCGTCTG |
| | | CACAATCTGCAAATCAAAGATAAGGGTATCTACCAGT |
| | | GCATTATCCACCATAAGAAGCCGACGGGTATGGTGAA |
| | | GATTCACGAGATGAACTCCGAGTTGTCTGTCCTGGCG |
| 35 | 1039 | GCCCCCCTCAAAATCCAAGCGTACTTCAACGAAACTG |
| | | CAGACTTACCGTGTCAGTTTGCCAATTCGCAGAATCT |
| | | GAGCCTGAGCGAACTGGTGGTTTTCTGGCAGGATCAG |
| | | GAGAACCTGGTTCTGAACGAAGTCTATCTGGGCAAAG |
| | | AGAAATTCGACAGCGTGAGTAGCAAGTATATGGGCCG |
| | | CACCAGCTTTGATAGCGACAGCTGGACCCTGCGTCTG |
| | | CACAATCTGCAAATCAAAGATAAGGGTATCTACCAGT |
| | | GCATTATCCACCATAAGAAGCCGAGCGGTATGGTGAA |
| | | GATTCACCAAATGGACTCCGAGTTGTCTGTCCTGGCG |
| 36 | 1040 | GCCCCCCTCAAAATCCAAGCGTACTTCAACGAAACTG |
| | | CAGACTTACCGTGTCAGTTTGCCAATTCGCAGAATCT |
| | | GAGCCTGAGCGAACTGGTGGTTTTCTGGCAGGATCAG |
| | | GAGAACCTGGTTCTGAACGAAGTCTATCTGGGCAAAG |
| | | AGCGGTTCGACAGCGTGGACAGCAAGTATATGGGCCG |
| | | CACCAGCTTTGATAGCGACAGCTGGACCCTGCGTCTG |
| | | CACAATCTGCAAATCAAAGATAAGGGTAGGTACCAGT |
| | | GCATTATCCACCATAAGAAGCCGACGGGTATGATTAA |
| | | TATTCACCAAATGAACTCCGAGTTGTCTGTCCTGGCG |
| 37 | 1041 | GCCCCCCTCAAAATCCAAGCGTACCTCAACGAAACTG |
| | | CAGACTTACCGTGTCAGTTTGCCAATTCGCAGAATCT |
| | | GAGCCTGAGCGAACTGGTGGTTTTCTGGCAGGATCAG |
| | | GAGAACCTGGTTCTGAACGAAGTCTATCTGGGCAAAG |
| | | AGAAATTCGACAGCGTGGACAGCAAGTATATGGGCCG |
| | | CACCAGCTTTGATAGCGACAGCTGGACCCTGCGTCTG |
| | | CACAATCTGCAAATCAAAGATAAGGGTATCTACCAGT |
| | | GCATTATCCACCATAAGAAGCCGACGGGTCTGGTGAA |
| | | GATTCACGAGATGAACTCCGAGTTGTCTGTCCTGGCG |
| 38 | 1042 | GCCCCCCTCAAAATCCAAGCGTACTTCAACGAAACTG |
| | | CAGACTTACCGTGTCAGTTTGCCAATTCGCAGAATCT |
| | | GAGCCTGAGCGAACTGGTGGTTTTCTGGCAGGATCAG |
| | | GAGAACCTGGTTCTGAACGAAGTCTATCTGGGCAAAG |
| | | AGATTTTCGACAGCGTGAGTAGCAAGTATATGGGCCG |
| | | CACCAGCTTTGATAGTGACAGCTGGACCCTGCGTCTG |
| | | CACAATCTGCAAATCAAAGATAAGGGTATCTACCAGT |
| | | GCATTATCCACCATAAGAAGCCGAGCGGTATGGTGAA |
| | | GATTCACCAAATGGACTCCGAGTTGTCTGTCCTGGCG |
| 39 | 1043 | GCCCCCCTCAAAATCCAAGCGTACTTCAACGAAACTG |
| | | CAGACTTACCGTGTCAGTTTGCCAATTCGCAGAATCT |
| | | GAGCCTGAGCGAACTGGTGGTTTTCTGGCAGGATCAG |
| | | GAGAACCTGGTTCTGAACGAAGTCTATCTGGGCAAAG |
| | | AGAAATTCGACAGCGTGGATAGCAAGTATATGGGCCG |
| | | CACCAGCTTTGATAGCGACAGCTGGACCCTGCGTCTG |
| | | CACAATCTGCAAATCAAAGATAAGGGTATCTACCAGT |
| | | GCATTATCCACCATAAGAAGCCGACGGGTATGATTAA |
| | | GATTCACGAGATGAACTCCGAGTTGTCTGTCCTGGCG |
| 40 | 1044 | GCCCCCCTCAAAATCCAAGCGTACTTCAACGAAACTG |
| | | CAGACTTACCGTGTCAGTTTGCCAATTCGCAGAATCT |
| | | GACCCTGAGCGAACTGGTGGTTTTCTGGCAGGATCAG |
| | | GAGAACCTGGTTCTGAACGAAGTCTATCTGGGCAAAG |
| | | AGAAATTCGACAGCGTGTCTAGCAAGTATATGGGCCG |
| | | CACCAGCTTTGATAGCGACAGCTGGACCCTGCGTCTG |
| | | CACAATCTGCAAATCAAAGATAAGGGTATCTACCAGT |
| | | GCATTATCCACCATAAGAAGCCGACGGGTATGATTAA |
| | | GATTCACGAGATGAGCTCCGAGTTGTCTGTCCTGGCG |
| 41 | 1045 | GCCCCCCTCAAAATCCAAGCGTACTTCAACGAAACTG |
| | | CAGACTTACCGTGTCAGTTTGCCAATTCGCAGAATCT |
| | | GACCCTGAGCGAACTGGTGGTTTTCTGGCAGGATCAG |
| | | GAGAACCTGGTTCTGAACGAAGTCTATCTGGGCAAAG |
| | | AGAAATTCGACAGCGTGGACAGCAAGTATATGGGCCG |
| | | CACCAGCTTTGATAGCGACAGCTGGACCCTGCGTCTG |
| | | CACAATCTGCAAATCAAAGATAAGGGTCTGTACCAGT |
| | | GCATTATCCACCATAAGAAGCCGACGGGTCTGGTGAA |
| | | GATTCACGAGATGAACTCCGAGTTGTCTGTCCTGGCG |
| 42 | 1046 | GCCCCCCTCAAAATCCAAGCGTACTTCAACGAAACTG |
| | | CAGACTTACCGTGTCAGTTTGCCAATTCGCAGAATCA |
| | | AAGCCTGAGCGAACTGGTGGTTTTCTGGCAGGATCAG |
| | | GAGAACCTGGTTCTGAACGAAGTCTATCTGGGCAAAG |
| | | AGAAATTCGACAGCGTGGACAGCAAGTATATGGGCCG |
| | | CACCAGCTTTGATAGCGACAGCTGGACCCTGCGTCTG |
| | | CACAATCTGCAAATCGAAGATAAGGGTATCTACCAGT |
| | | GCATTATCCACCATAAGAAGCCGAGCGGTATGGTGAA |
| | | GATTCACCAAATGGACTCCGAGTTGTCTGTCCTGGCG |
| 43 | 1047 | GCCCCCCTCAAAATCCAAGCGTACTTCAACGAAACTG |
| | | CAGACTTACCGTGTCAGTTTGCCAATTCGCAGAATCT |
| | | GAGCCTGAGCGAACTGGTGGTTTTCTGGCAGGATCAG |
| | | GAGAACCTGGTTCTGAACGAAGTCTATCTGGGCAAAG |
| | | AGAAATTCGACAGCGTGGACAGCAAGTATATGGGCCG |
| | | CACCAGCTTTGATAGCGACAGCTGGACCCTGCGTCTG |
| | | CACAATCTGCAAATCAAAGATAAGGGTATCTACCAGT |
| | | GCATTATCCACCATAAGAAGCCGACGGGTCTGGTGAA |
| | | GATTCACGAGATGAACTCCGAGTTGTCTGTCCTGGCG |

Other Sequences

(human CTLA-4)
SEQ ID NO: 1
MHVAQPAVVLASSRGIASFVCEYASPGKATEVRVTVLRQADSQVTEVCAATYMMGNELTFLDDSI
CTGTSSGNQVNLTIQGLRAMDTGLYICKVELMYPPPYYLGIGNGTQIYVIAKEKKPSYNRGLCEN
APNRARM
(human CD28)
SEQ ID NO: 2
MLRLLLALNLFPSIQVTGNKILVKQSPMLVAYDNAVNLSCKYSYNLFSREFRASLHKGLDSAVEV
CVVYGNYSQQLQVYSKTGFNCDGKLGNESVTFYLQNLYVNQTDIYFCKIEVMYPPPYLDNEKSNG
TIIHVKGKHLCPSPLFPGPSKPFWVLVVVGGVLACYSLLVTVAFIIFWVRSKRSRLLHSDYMNMT
PRRPGPTRKHYQPYAPPRDFAAYRS
SEQ ID NO: 3
APLKIQAYFNETADLPCQFANSQNQSLSELVVFWQDQENLVLNEVYLGKEKFDSVHSKYMGRTSF
DSDSWTLRLHNLQIKDKGLYQCIIHHKKPTGMIRIHQMNSELSVLA
SEQ ID NO: 4
MDPQCTMGLSNILFVMAFLLSGAAPLKIQAYFNETADLPCQFANSQNQSLSELVVFWQDQENLVL
NEVYLGKEKFDSVHSKYMGRTSFDSDSWTLRLHNLQIKDKGLYQCIIHHKKPTGMIRIHQMNSEL
SVLANFSQPEIVPISNITENVYINLTCSSIHGYPEPKKMSVLLRTKNSTIEYDGIMQKSQDNVTE
LYDVSISLSVSFPDVTSNMTIFCILETDKTRLLSSPFSIELEDPQPPPDHIP
SEQ ID NO: 5
APLKIQAYFNETADLPCQFANSQNQSLSELVVFWQDQENLVLNEVYLGKEKFDSVASKYMGRTSF
DSDSWTLRLHNLQIKDKGLYQCIIHHKKPTGMIRIHQMNSELSVLA
(human CD86)
SEQ ID NO: 44
MDPQCTMGLSNILFVMAFLLSGAAPLKIQAYFNETADLPCQFANSQNQSLSELVVFWQDQENLVL
NEVYLGKEKFDSVHSKYMGRTSFDSDSWTLRLHNLQIKDKGLYQCIIHHKKPTGMIRIHQMNSEL
SVLANFSQPEIVPISNITENVYINLTCSSIHGYPEPKKMSVLLRTKNSTIEYDGIMQKSQDNVTE
LYDVSISLSVSFPDVTSNMTIFCILETDKTRLLSSPFSIELEDPQPPPDHIPWITAVLPTVIICV
MVFCLILWKWKKKKRPRNSYKCGTNTMEREESEQTKKREKIHIPERSDEAQRVFKSSKTSSCDKS
DTCF
(murine CTLA-4)
SEQ ID NO: 45
MACLGLRRYKAQLQLPSRTWPFVALLTLLFIPVFSEAIQVTQPSVVLASSHGVASFPCEYSPSHN
TDEVRVTVLRQTNDQMTEVCATTFTEKNTVGFLDYPFCSGTFNESRVNLTIQGLRAVDTGLYLCK
VELMYPPPYFVGMGNGTQIYVIDPEPCPDSDELLWILVAVSLGLFFYSFLVSAVSLSKMLKKRSP
LTTGVYVKMPPTEPECEKQFQPYFIPIN
(murine CD28)
SEQ ID NO: 46
MTLRLLFLALNFFSVQVTENKILVKQSPLLVVDSNEVSLSCRYSYNLLAKEFRASLYKGVNSDVE
VCVGNGNFTYQPQFRSNAEFNCDGDFDNETVTFRLWNLHVNHTDIYFCKIEFMYPPPYLDNERSN
GTIIHIKEKHLCHTQSSPKLFWALVVVAGVLFCYGLLVTVALCVIWTNSRRNRLLQVTTMNMTPR
RPGLTRKPYQPYAPARDFAAYRP
(human OX40)
SEQ ID NO: 51
MCVGARRLGRGPCAALLLLGLGLSTVTGLHCVGDTYPSNDRCCHECRPGNGMVSRCSRSQNTVCR
PCGPGFYNDVVSSKPCKPCTWONLRSGSERKQLCTATQDTVCRCRAGTQPLDSYKPGVDCAPCPP
GHFSPGDNQACKPWTNCTLAGKHTLQPASNSSDAICEDRDPPATQPQETQGPPARPITVQPTEAW
PRTSQGPSTRPVEVPGGRAVAAILGLGLVLGLLGPLAILLALYLLRRDQRLPPDAHKPPGGGSFR
TPIQEEQADAHSTLAKI
SEQ ID NO: 140
gcttccacca agggcccatc cgtcttcccc ctggcgccct gctccaggag cacctccgag
agcacagccg ccctgggctg cctggtcaag gactacttcc ccgaaccggt gacggtgtcg
tggaactcag gcgccctgac cagcggcgtg cacaccttcc cggctgtcct acagtcctca
ggactctact ccctcagcag cgtggtgacc gtgccctcca gcagcttggg cacgaagacc
tacacctgca acgtagatca caagcccagc aacaccaagg tggacaagag agttgagtcc
aaatatggtc ccccatgccc accttgccca gcacctgagt tcctgggggg accatcagtc
ttcctgttcc ccccaaaacc caaggacact ctcatgatct cccggacccc tgaggtcacg
tgcgtggtgg tggacgtgag ccaggaagac cccgaggtcc agttcaactg gtacgtggat
ggcgtggagg tgcataatgc caagacaaag ccgcgggagg agcagttcaa cagcacgtac
cgtgtggtca gcgtcctcac cgtcctgcac caggactggc tgaacggcaa ggagtacaag
tgcaaggtct ccaacaaagg cctcccgtcc tccatcgaga aaaccatctc caaagccaaa
gggcagcccc gagagccaca ggtgtacacc ctgcccccat cccaggagga gatgaccaag
aaccaggtca gcctgacctg cctggtcaaa ggcttctacc ccagcgacat cgccgtggag
tgggagagca atgggcagcc ggagaacaac tacaagacca cgcctcccgt gctggactcc
gacggctcct tcttcctcta cagcaggcta accgtggaca agagcaggtg gcaggagggg
aatgtcttct catgctccgt gatgcatgag gctctgcaca accgctacac acagaagagc
ctctccctgt ctctgggtaa a
SEQ ID NO: 141
agctttctgg ggcaggccgg gcctgacttt ggctgggggc agggaggggg ctaaggtgac
gcaggtggcg ccagccaggt gcacacccaa tgcccatgag cccagacact ggaccctgca
tggaccatcg cggatagaca agaaccgagg ggcctctgcg ccctgggccc agctctgtcc
cacaccgcgg tcacatggca ccacctctct tgcagcttcc accaagggcc catccgtctt
ccccctggcg ccctgctcca ggagcacctc cgagagcaca gccgccctgg gctgcctggt
caaggactac ttccccgaac cggtgacggt gtcgtggaac tcaggcgccc tgaccagcgg
cgtgcacacc ttcccggctg tcctacagtc ctcaggactc tactccctca gcagcgtggt
gaccgtgccc tccagcagct tgggcacgaa gacctacacc tgcaacgtag atcacaagcc
cagcaacacc aaggtggaca agagagttgg tgagaggcca gcacagggag ggagggtgtc
tgctggaagc caggctcagc cctcctgcct ggacgcaccc cggctgtgca gccccagccc
agggcagcaa ggcatgcccc atctgtctcc tcacccggag gcctctgacc accccactca
tgctcaggga gagggtcttc tggatttttc caccaggctc ccggcaccac aggctggatg
cccctacccc aggccctgcg catacagggc aggtgctgcg ctcagacctg ccaagagcca
tatccgggag gaccctgccc ctgacctaag cccaccccaa aggccaaact ctccactccc
tcagctcaga caccttctct cctcccagat ctgagtaact cccaatcttc tctctgcaga
gtccaaatat ggtcccccat gcccaccttg cccaggtaag ccaacccagg cctcgccctc
cagctcaagg cgggacaggt gccctagagt agcctgcatc cagggacagg ccccagccgg
gtgctgacgc atccacctcc atctcttcct cagcacctga gttcctgggg ggaccatcag
tcttcctgtt ccccccaaaa cccaaggaca ctctcatgat ctcccggacc cctgaggtca
cgtgcgtggt ggtggacgtg agccaggaag accccgaggt ccagttcaac tggtacgtgg
atggcgtgga ggtgcataat gccaagacaa agccgcggga ggagcagttc aacagcacgt
accgtgtggt cagcgtcctc accgtcctgc accaggactg gctgaacggc aaggagtaca
agtgcaaggt ctccaacaaa ggcctcccgt cctccatcga gaaaaccatc tccaaagcca
aaggtgggac ccacggggtg cgagggccac acggacagag gccagctcgg cccaccctct
gccctgggag tgaccgctgt gccaacctct gtccctacag ggcagccccg agagccacag
gtgtacaccc tgcccccatc ccaggaggag atgaccaaga accaggtcag cctgacctgc
ctggtcaaag gcttctaccc cagcgacatc gccgtggagt gggagagcaa tgggcagccg
gagaacaact acaagaccac gcctcccgtg ctggactccg acggctcctt cttcctctac
agcaggctaa ccgtggacaa gagcaggtgg caggagggga atgtcttctc atgctccgtg
atgcatgagg ctctgcacaa ccgctacaca cagaagagcc tctocctgtc tctgggtaaa
tgagtgccag ggccggcaag cccccgctcc ccgggctctc ggggtcgcgc gaggatgctt
ggcacgtacc ccgtctacat acttcccagg cacccagcat ggaaataaag cacccaccac
tgccctgggc ccctgtgaga ctgtgatggt tctttccacg ggtcaggccg agtctgaggc
ctgagtgaca tgagggaggc agagcgggtc ccactgtccc cacactgg
SEQ ID NO: 142
gcttccacca agggcccatc cgtcttcccc ctggcgccct gctccaggag cacctccgag
agcacagccg ccctgggctg cctggtcaag gactacttcc ccgaaccggt gacggtgtcg
tggaactcag gcgccctgac cagcggcgtg cacaccttcc cggctgtcct acagtcctca
ggactctact ccctcagcag cgtggtgacc gtgccctcca gcagcttggg cacgaagacc
tacacctgca acgtagatca caagcccagc aacaccaagg tggacaagag agttgagtcc
aaatatggtc ccccatgccc atcatgccca gcacctgagt tcctgggggg accatcagtc
ttcctgttcc ccccaaaacc caaggacact ctcatgatct cccggacccc tgaggtcacg
tgcgtggtgg tggacgtgag ccaggaagac cccgaggtcc agttcaactg gtacgtggat
ggcgtggagg tgcataatgc caagacaaag ccgcgggagg agcagttcaa cagcacgtac
cgtgtggtca gcgtcctcac cgtcctgcac caggactggc tgaacggcaa ggagtacaag
tgcaaggtct ccaacaaagg cctcccgtcc tccatcgaga aaaccatctc caaagccaaa
gggcagcccc gagagccaca ggtgtacacc ctgcccccat cccaggagga gatgaccaag
aaccaggtca gcctgacctg cctggtcaaa ggcttctacc ccagcgacat cgccgtggag
tgggagagca atgggcagcc ggagaacaac tacaagacca cgcctcccgt gctggactcc
gacggctcct tcttcctcta cagcaggcta accgtggaca agagcaggtg gcaggagggg
aatgtcttct catgctccgt gatgcatgag gctctgcaca accactacac acagaagagc
ctctccctgt ctctgggtaa a
SEQ ID NO: 143
agctttctgg ggcaggccgg gcctgacttt ggctgggggc agggaggggg ctaaggtgac
gcaggtggcg ccagccaggt gcacacccaa tgcccatgag cccagacact ggaccctgca
tggaccatcg cggatagaca agaaccgagg ggcctctgcg ccctgggccc agctctgtcc
cacaccgcgg tcacatggca ccacctctct tgcagcttcc accaagggcc catccgtctt
ccccctggcg ccctgctcca ggagcacctc cgagagcaca gccgccctgg gctgcctggt
caaggactac ttccccgaac cggtgacggt gtcgtggaac tcaggcgccc tgaccagcgg
cgtgcacacc ttcccggctg tcctacagtc ctcaggactc tactccctca gcagcgtggt
gaccgtgccc tccagcagct tgggcacgaa gacctacacc tgcaacgtag atcacaagcc
cagcaacacc aaggtggaca agagagttgg tgagaggcca gcacagggag ggagggtgtc
tgctggaagc caggctcagc cctcctgcct ggacgcaccc cggctgtgca gccccagccc
agggcagcaa ggcatgcccc atctgtctcc tcacccggag gcctctgacc accccactca
tgctcaggga gagggtcttc tggatttttc caccaggctc ccggcaccac aggctggatg
cccctacccc aggccctgcg catacagggc aggtgctgcg ctcagacctg ccaagagcca
tatccgggag gaccctgccc ctgacctaag cccaccccaa aggccaaact ctccactccc
tcagctcaga caccttctct cctcccagat ctgagtaact cccaatcttc tctctgcaga
gtccaaatat ggtcccccat gcccatcatg cccaggtaag ccaacccagg cctcgccctc
cagctcaagg cgggacaggt gccctagagt agcctgcatc cagggacagg ccccagccgg
gtgctgacgc atccacctcc atctcttcct cagcacctga gttcctgggg ggaccatcag
tcttcctgtt ccccccaaaa cccaaggaca ctctcatgat ctcccggacc cctgaggtca
cgtgcgtggt ggtggacgtg agccaggaag accccgaggt ccagttcaac tggtacgtgg
atggcgtgga ggtgcataat gccaagacaa agccgcggga ggagcagttc aacagcacgt
accgtgtggt cagcgtcctc accgtcctgc accaggactg gctgaacggc aaggagtaca
agtgcaaggt ctccaacaaa ggcctcccgt cctccatcga gaaaaccatc tccaaagcca
aaggtgggac ccacggggtg cgagggccac acggacagag gccagctcgg cccaccctct
gccctgggag tgaccgctgt gccaacctct gtccctacag ggcagccccg agagccacag
gtgtacaccc tgcccccatc ccaggaggag atgaccaaga accaggtcag cctgacctgc
ctggtcaaag gcttctaccc cagcgacatc gccgtggagt gggagagcaa tgggcagccg
gagaacaact acaagaccac gcctcccgtg ctggactccg acggctcctt cttcctctac
agcaggctaa ccgtggacaa gagcaggtgg caggagggga atgtottctc atgctccgtg
atgcatgagg ctctgcacaa ccactacaca cagaagagcc tctccctgtc tctgggtaaa
tgagtgccag ggccggcaag cccccgctcc ccgggctctc ggggtcgcgc gaggatgctt
ggcacgtacc ccgtctacat acttcccagg cacccagcat ggaaataaag cacccaccac
tgccctgggc ccctgtgaga ctgtgatggt tctttccacg ggtcaggccg agtctgaggc
ctgagtgaca tgagggaggc agagcgggtc ccactgtccc cacactgg
SEQ ID NO: 145
gcctccacca agggcccatc ggtcttcccc ctggcaccct cctccaagag cacctctggg
ggcacagcgg ccctgggctg cctggtcaag gactacttcc ccgaaccggt gacggtgtcg
tggaactcag gcgccctgac cagcggcgtg cacaccttcc cggctgtcct acagtcctca
ggactctact ccctcagcag cgtggtgacc gtgccctcca gcagcttggg cacccagacc
tacatctgca acgtgaatca caagcccagc aacaccaagg tggacaagaa agttgagccc
aaatcttgtg acaaaactca cacatgccca ccgtgcccag cacctgaact cctgggggga
ccgtcagtct tcctcttccc cccaaaaccc aaggacaccc tcatgatctc ccggacccct
gaggtcacat gcgtggtggt ggacgtgagc cacgaagacc ctgaggtcaa gttcaactgg
tacgtggacg gcgtggaggt gcataatgcc aagacaaagc cgcgggagga gcagtacaac
agcacgtacc gtgtggtcag cgtcctcacc gtcctgcacc aggactggct gaatggcaag
gagtacaagt gcaaggtctc caacaaagcc ctcccagccc ccatcgagaa aaccatctcc
aaagccaaag ggcagccccg agaaccacag gtgtacaccc tgcccccatc ccgggatgag
ctgaccaaga accaggtcag cctgacctgc ctggtcaaag gcttctatcc cagcgacatc
gccgtggagt gggagagcaa tgggcagccg gagaacaact acaagaccac gcctcccgtg
ctggactccg acggctcctt cttcctctac agcaagctca ccgtggacaa gagcaggtgg
cagcagggga acgtcttctc atgctccgtg atgcatgagg ctctgcacaa ccactacacg
cagaagagcc tctccctgtc tccgggtaaa
SEQ ID NO: 146
gcctccacca agggcccatc ggtcttcccc ctggcaccct cctccaagag cacctctggg
ggcacagcgg ccctgggctg cctggtcaag gactacttcc ccgaaccggt gacggtgtcg
tggaactcag gcgccctgac cagcggcgtg cacaccttcc cggctgtcct acagtcctca
ggactctact ccctcagcag cgtggtgacc gtgccctcca gcagcttggg cacccagacc
tacatctgca acgtgaatca caagcccagc aacaccaagg tggacaagaa agttggtgag
aggccagcac agggagggag ggtgtctgct ggaagccagg ctcagcgctc ctgcctggac
gcatcccggc tatgcagccc cagtccaggg cagcaaggca ggccccgtct gcctcttcac
ccggaggcct ctgcccgccc cactcatgct cagggagagg gtcttctggc tttttcccca
ggctctgggc aggcacaggc taggtgcccc taacccaggc cctgcacaca aaggggcagg
tgctgggctc agacctgcca agagccatat ccgggaggac cctgcccctg acctaagccc
accccaaagg ccaaactctc cactccctca gctcggacac cttctctcct cccagattcc
agtaactccc aatcttctct ctgcagagcc caaatcttgt gacaaaactc acacatgccc
accgtgccca ggtaagccag cccaggcctc gccctccagc tcaaggcggg acaggtgccc
tagagtagcc tgcatccagg gacaggcccc agccgggtgc tgacacgtcc acctccatct
cttcctcagc acctgaactc ctggggggac cgtcagtctt cctcttcccc ccaaaaccca
aggacaccct catgatctcc cggacccctg aggtcacatg cgtggtggtg gacgtgagcc
acgaagaccc tgaggtcaag ttcaactggt acgtggacgg cgtggaggtg cataatgcca
agacaaagcc gcgggaggag cagtacaaca gcacgtaccg tgtggtcagc gtcctcaccg
tcctgcacca ggactggctg aatggcaagg agtacaagtg caaggtctcc aacaaagccc
tcccagcccc catcgagaaa accatctcca aagccaaagg tgggacccgt ggggtgcgag
ggccacatgg acagaggccg gctcggccca ccctctgccc tgagagtgac cgctgtacca
acctctgtcc ctacagggca gccccgagaa ccacaggtgt acaccctgcc cccatcccgg
gatgagctga ccaagaacca ggtcagcctg acctgcctgg tcaaaggctt ctatcccagc
gacatcgccg tggagtggga gagcaatggg cagccggaga acaactacaa gaccacgcct
cccgtgctgg actccgacgg ctccttcttc ctctacagca agctcaccgt ggacaagagc
aggtggcagc aggggaacgt cttctcatgc tccgtgatgc atgaggctct gcacaaccac
tacacgcaga agagcctctc cctgtctccg ggtaaa
SEQ ID NO: 147
cgaactgtgg ctgcaccatc tgtcttcatc ttcccgccat ctgatgagca gttgaaatct
ggaactgcct ctgttgtgtg cctgctgaat aacttctatc ccagagaggc caaagtacag
tggaaggtgg ataacgccct ccaatcgggt aactcccagg agagtgtcac agagcaggac
agcaaggaca gcacctacag cctcagcagc accctgacgc tgagcaaagc agactacgag
aaacacaaag tctacgcctg cgaagtcacc catcagggcc tgagctcgcc cgtcacaaag
agcttcaaca ggggagagtg t
SEQ ID NO: 148
gcttccacca agggcccatc cgtcttcccc ctggcgccct gctccaggag cacctccgag
agcacagccg ccctgggctg cctggtcaag gactacttcc ccgaaccggt gacggtgtcg
tggaactcag gcgccctgac cagcggcgtg cacaccttcc cggctgtcct acagtcctca
ggactctact ccctcagcag cgtggtgacc gtgccctcca gcagcttggg cacgaagacc
tacacctgca acgtagatca caagcccagc aacaccaagg tggacaagag agttgagtcc
aaatatggtc ccccatgccc accttgccca gcacctgagt tcctgggggg accatcagtc
ttcctgttcc ccccaaaacc caaggacact ctcatgatct cccggacccc tgaggtcacg
tgcgtggtgg tggacgtgag ccaggaagac cccgaggtcc agttcaactg gtacgtggat
ggcgtggagg tgcataatgc caagacaaag ccgcgggagg agcagttcaa cagcacgtac
cgtgtggtca gcgtcctcac cgtcctgcac caggactggc tgaacggcaa ggagtacaag
tgcaaggtct ccaacaaagg cctcccgtcc tccatcgaga aaaccatctc caaagccaaa
gggcagcccc gagagccaca ggtgtacacc ctgcccccat cccaggagga gatgaccaag
aaccaggtca gcctgacctg cctggtcaaa ggcttctacc ccagcgacat cgccgtggag
tgggagagca atgggcagcc ggagaacaac tacaagacca cgcctcccgt gctggactcc
gacggctcct tcttcctcta cagcaggcta accgtggaca agagcaggtg gcaggagggg
aatgtcttct catgctccgt gatgcatgag gctctgcaca accactacac acagaagagc
ctctccctgt ctctgggtaa a
SEQ ID NO: 149
agctttctgg ggcaggccgg gcctgacttt ggctgggggc agggaggggg ctaaggtgac
gcaggtggcg ccagccaggt gcacacccaa tgcccatgag cccagacact ggaccctgca
tggaccatcg cggatagaca agaaccgagg ggcctctgcg ccctgggccc agctctgtcc
cacaccgcgg tcacatggca ccacctctct tgcagcttcc accaagggcc catccgtctt
ccccctggcg ccctgctcca ggagcacctc cgagagcaca gccgccctgg gctgcctggt
caaggactac ttccccgaac cggtgacggt gtcgtggaac tcaggcgccc tgaccagcgg
cgtgcacacc ttcccggctg tcctacagtc ctcaggactc tactccctca gcagcgtggt
gaccgtgccc tccagcagct tgggcacgaa gacctacacc tgcaacgtag atcacaagcc
cagcaacacc aaggtggaca agagagttgg tgagaggcca gcacagggag ggagggtgtc
tgctggaagc caggctcagc cctcctgcct ggacgcaccc cggctgtgca gccccagccc
agggcagcaa ggcatgcccc atctgtctcc tcacccggag gcctctgacc accccactca
tgctcaggga gagggtcttc tggatttttc caccaggctc ccggcaccac aggctggatg
cccctacccc aggccctgcg catacagggc aggtgctgcg ctcagacctg ccaagagcca
tatccgggag gaccctgccc ctgacctaag cccaccccaa aggccaaact ctccactccc
tcagctcaga caccttctct cctcccagat ctgagtaact cccaatcttc tctctgcaga
gtccaaatat ggtcccccat gcccaccttg cccaggtaag ccaacccagg cctcgccctc
cagctcaagg cgggacaggt gccctagagt agcctgcatc cagggacagg ccccagccgg
gtgctgacgc atccacctcc atctcttcct cagcacctga gttcctgggg ggaccatcag
tcttcctgtt ccccccaaaa cccaaggaca ctctcatgat ctcccggacc cctgaggtca
cgtgcgtggt ggtggacgtg agccaggaag accccgaggt ccagttcaac tggtacgtgg
atggcgtgga ggtgcataat gccaagacaa agccgcggga ggagcagttc aacagcacgt
accgtgtggt cagcgtcctc accgtcctgc accaggactg gctgaacggc aaggagtaca
agtgcaaggt ctccaacaaa ggcctcccgt cctccatcga gaaaaccatc tccaaagcca
aaggtgggac ccacggggtg cgagggccac acggacagag gccagctcgg cccaccctct
gccctgggag tgaccgctgt gccaacctct gtccctacag ggcagccccg agagccacag
gtgtacaccc tgcccccatc ccaggaggag atgaccaaga accaggtcag cctgacctgc
ctggtcaaag gcttctaccc cagcgacatc gccgtggagt gggagagcaa tgggcagccg
gagaacaact acaagaccac gcctcccgtg ctggactccg acggctcctt cttcctctac
agcaggctaa ccgtggacaa gagcaggtgg caggagggga atgtcttctc atgctccgtg
atgcatgagg ctctgcacaa ccactacaca cagaagagcc tctccctgtc tctgggtaaa
tgagtgccag ggccggcaag cccccgctcc ccgggctctc ggggtcgcgc gaggatgctt
ggcacgtacc ccgtctacat acttcccagg cacccagcat ggaaataaag cacccaccac
tgccctgggc ccctgtgaga ctgtgatggt tctttccacg ggtcaggccg agtctgaggc
ctgagtgaca tgagggaggc agagcgggtc ccactgtccc cacactgg

### EXAMPLES

The present invention is further illustrated by the following examples which should not be construed as further limiting. The contents of all figures and all references, patents and published patent applications cited throughout this application are expressly incorporated herein by reference.

### Example 1—CTLA-4 Binding Domains

CTLA-4 binding domain polypeptides were selected and expressed as described in WO 2014/207063 (see Examples) and were assayed for binding to CTLA-4 by at least one of ELISA and surface plasmon resonance as described below.

Binding ELISA

96-well flat bottom high binding plates (Greiner, #655074) were coated with either CTLA4-Fc (Fitzgerald, #30R-CD152) or CD28-Fc (R&D systems, 342-CD) by incubating overnight at 4° C. The plates were washed (Wash buffer: PBS+0.05% Tween 20 (PBST) Medicago, #09-9410-100) and then blocked in PBST+3% BSA (Merck, #1.12018.0100). The plates were washed again and sample or controls (serially diluted 1/5 from 200-0.001 μg/ml) were added to the wells. The samples were incubated for 1 h at room temperature and then washed. Detection antibody, goat-anti-human IgG Fcγ-HRP (Jackson, #109-035-098) was added and the plates were subsequently developed using SuperSignal Pico Chemiluminescent substrate (Thermo Scientific, #37069) and detected with an Envision reader (Perkin Elmer). EC50 values were calculated for both CTLA4 and CD28. The binding ratio (EC50 binding ratio=[EC50 for CD28]÷[EC50 for CTLA-4]) was calculated for each polypeptide and is shown in Table 1.1.

Surface Plasmon Resonance

Either CTLA4-Fc (Fitzgerald, #30R-CD152) or CD28-Fc (R&D Systems, 342-CD) was immobilized to the Biacore™ sensor chip, CM5, using conventional amine coupling. The CD86 mutant molecules and controls (serially diluted 1/2 100-1.5 nM) were analyzed for binding in HBS-P (GE, BR-1003-68) at a flow rate of 30 μl/ml. The association was followed for 3 minutes and the dissociation for 10 minutes. Regeneration was performed twice using 5 mM NaOH for 30 seconds. The kinetic parameters and the affinity constants were calculated using BIAevaluation 4.1 software (Table 1.3).

Inhibition ELISA

96-well flat bottom plate high binding plates (Greiner, #655074) were coated with wildtype CD86-Fc (R&D Systems, #7625-B2) by incubating overnight at 4° C. The plates were washed (Wash buffer: PBS+0.05% Tween 20 (PBST) Medicago, #09-9410-100) and then blocked in PBST+3% BSA (Merck, #1.12018.0100). The sample (CD86 mutant or wild type protein; serially diluted 1/4 from 30000 to 0.3 ng/ml) was incubated with biotinylated-CTLA4 (Fitzgerald, #30R-CD152) in room temperature 1 h, the mixture was then added to the blocked wells in the ELISA plate. Detection was performed with Streptavidin-HRP (Pierce, #21126) and the plates were subsequently developed using SuperSignal Pico Chemiluminescent substrate (Thermo Scientific, #37069) and detected with Envision reader (Perkin Elmer). The results are shown in FIG. 2. IC50 values were calculated and are shown in the tables below. All molecules tested showed better IC50 value than both wild type and H79A, the IC of the best mutant CD86 molecule was improved over 100-fold compared to wild type. Results for exemplary molecules 900, 901, 904, 906, 907, 908, 910, 915 and 938 are shown in Table 1.1. Kd binding ratio=[Kd for CD28]÷[Kd for CTLA-4]. The full amino acid sequences for exemplary molecules 900, 901, 904, 906, 907, 908, 910, 915 and 938 are provided as SEQ ID NOs: 6 to 14, respectively.

TABLE 1.1

| | Mutated positions and amino acid change | EC50 | |
|---|---|---|---|
| | relative to wild-type | binding | Kd binding |
| Sample | (positions numbered as in FIG. 4) | ratio | ratio |
| 900 | H79D, L107I, I111V, T118S, M120L, I121V, R122K, | 3.5 | ND* |
| | Q125E | | |
| 901 | Q48L, S49T, L107I, I111V, R122K, Q125E, A134T | 17.2 | 2.7 |
| 904 | V54I, K74R, S77A, H79D, L107I, T118S, I121V, R122K, | 12.2 | 6.8 |
| | N127D | | |
| 906 | F32I, Q48L, K74R, H79D, L107F, | 16.2 | 0.8 |
| | M120L, I121V, R122K, Q125E | | |
| 907 | R122K, Q125E | 30.5 | 5.6 |
| 908 | L107I, I121V, R122K, Q125E | 6.2 | 4.7 |
| 910 | H79D, L107I, I121V, R122K, Q125E | 7.7 | 5.1 |
| 915 | V64I, H79D, L107F, T118S, M120L, R122K, N127D | 9.9 | 1.9 |
| 938 | V64I, L107I, I121V, R122K | 2.0 | 5.5 |
| | Wild type | 3.4 | 1.6 |

*No detectable binding was seen in the BIAcore ™ analysis nor binding ELISA

Results for exemplary molecules 1038, 1039, 1040, 1041, 1042, 1043, 1044, 1045, 1046 and 1047 are shown in Tables 1.2 and 1.3, and in FIGS. 2 and 3. The full amino acid sequences for exemplary molecules 1038, 1039, 1040, 1041, 1042, 1043, 1044, 1045, 1046 and 1047 are provided as SEQ ID NOs: 15 to 24, respectively.

TABLE 1.2

| Sample | EC50 | Sample | IC50 |
|---|---|---|---|
| 1038 | 0.14 | — | — |
| 1039 | 0.039 | — | — |
| 1040 | 0.0076 | 1040 | 0.049 |
| 1041 | 0.087 | 1041 | 3.1 |
| 1042 | 0.29 | 1042 | 4.3 |
| 1043 | 0.035 | 1043 | 4.0 |
| 1044 | 0.029 | 1044 | 1.4 |
| 1045 | 0.047 | 1045 | 2.6 |
| 1046 | 0.019 | 1046 | 1.1 |
| 1047 | 0.037 | 1047 | 0.98 |
| Wild type | 0.51 | Wild type | 15 |
| Prior Art | 0.81 | H79A | 25 |
| Negative | No activity | Negative | No activity |
| control | | control | |

TABLE 1.3

| | Mutated positions and amino acid change | ka | kd | K_{D} |
|---|---|---|---|---|
| Sample | (positions numbered as in FIG. 4) | (1/Ms) | (1/s) | (nM) |
| 1038 | Q48L, H79D, L107I, I121V, R122K, Q125E | 1.0e6 | 0.012 | 12 |
| 1039 | Q48L, H79S, L107I, T118S, I121V, R122K, | 1.0e6 | 8.5e−3 | 8 |
| | N127D | | | |
| 1040 | Q48L, K74R, H79D, L107R, R122N | 1.0e6 | 3.2e−3 | 3 |
| 1041 | F32L, Q48L, H79D, L107I, M120L, I121V, | 7.0e5 | 8.4e−3 | 12 |
| | R122K, 125E | | | |
| 1042 | Q48L, K74I, H79S, L107I, T118S, I121V, | 4.4e5 | 0.011 | 25 |
| | R122K, N127D | | | |
| 1043 | Q48L, H79D, L107I, R122K, Q125E | 1.1e6 | 0.011 | 10 |
| 1044 | Q48L, S49T, H79S, L107I, R122K, Q125E, | 1.1e6 | 9.4e−3 | 8 |
| | N127S | | | |
| 1045 | Q48L, S49T, H79D, M120L, I121V, R122K, | 9.4e5 | 8.3e−3 | 9 |
| | Q125E | | | |
| 1046 | H79D, K103E, L107I, T118S, I121V, R122K, | 1.4e6 | 8.0e−3 | 6 |
| | N127D | | | |
| 1047 | Q48L, H79D, L107I, M120L, I121V, R122K, | 8.5e5 | 8.4e−3 | 10 |
| | Q125E | | | |
| Wild | | 4.6e5 | 0.023 | 50 |
| type | | | | |
| H79A | | 3.4e5 | 0.022 | 63 |

### Example 2—Cross-Reactivity to Murine CTLA-4 of Exemplary Polypeptide from Clone 1040

The relative affinity for murine and human CTLA-4 of an exemplary mutant CD86 molecule 1040 was investigated using an inhibition ELISA binding assay. The 1040 molecule used in these experiments was conjugated to an anti-CD40 antibody as part of a bispecific molecule. The CTLA-4 binding properties of the CD86 molecule are not affected by this conjugation (data not shown).

In brief, 96-well flat bottom plate high binding plates (Greiner #655074) were coated with human CTLA-4 (Fitzgerald) incubating overnight at 4° C. The plates were washed (Wash buffer: PBS+0.05% Tween 20 (PBST) Medicago #09-9410-100) and then blocked in PBST+3% BSA (Merck, #1.12018.0100).

The sample (exemplary CD86 mutant) was pre-incubated at room temperature for 1 hour with soluble biotinylated human CTLA4 (Fitzgerald #30R-CD152) or soluble murine CTLA-4 (R&D systems) at different concentrations (serial dilutions 1/4 from 30000 to 0.3 ng/ml).

The mixture was then added to the blocked wells in the ELISA plate. Detection was performed with Streptavidin-HRP (Pierce, #21126) and the plates were subsequently developed using SuperSignal Pico Chemiluminescent substrate (Thermo Scientific, #37069) and detected with Envision reader (Perkin Elmer). The results are shown in FIG. 5. The observed inhibition curves with murine and human CTLA-4 demonstrate that the binding affinity of the exemplary CD86 mutant (1040) to the two forms of CTLA-4 is of a similar magnitude. The other clones tested in Example 1 were also found to bind to murine CTLA-4 (data not shown).

### Example 3—Characterisation of OX40 Antibodies

Characteristics of exemplary OX40 antibodies are summarised in Table 3.1 below.

TABLE 3.1

| | | | | | huOX40 | | | | Association | Association |
|---|---|---|---|---|---|---|---|---|---|---|
| | CDR H3 | Dose | | M. mulatta | binding | | | Dissociation | rate | rate |
| | length | response | T-cell | OX40 | FACS | Hydropathy | Isoelectric | constant | constant | constant |
| Antibody | (IMGT) | ELISA (IgG) | agonist | binding | (CHO) | index | point | K_{D} (M) | ka (1/Ms) | kd (1/s) |
| 1166/1167 | 10 | <1 nM | Yes | Yes | Yes | −0.392 | 9.11 | 3.22E−10 | 9.01E+04 | 2.90E−05 |
| 1170/1171 | 10 | <1 nM | Yes | Yes | Yes | −0.415 | 9.11 | 2.50E−10 | 1.45E+06 | 3.63E−04 |
| 1164/1135 | 11 | <1 nM | Yes | Yes | Yes | −0.398 | 9.21 | 3.08E−10 | 2.51E+05 | 7.73E−05 |
| 1168/1135 | 11 | <1 nM | Yes | Yes | Yes | −0.404 | 9.19 | 3.27E−10 | 5.18E+05 | 1.69E−04 |
| 1482/1483 | 9 | <1 nM | Yes | Yes | Yes | −0.381 | 9.19 | 7.53E−10 | 7.76E+05 | 5.84E−04 |
| 1490/1135 | 11 | <1 nM | Yes | Yes | Yes | −0.407 | 9.18 | 3.07E−10 | 3.87E+06 | 1.19E−03 |
| 1514/1515 | 14 | <1 nM | Yes | Yes | Yes | −0.399 | 9.11 | 6.40E−10 | 2.57E+05 | 1.64E−04 |
| 1520/1135 | 17 | <1 nM | Yes | Yes | Yes | −0.394 | 9.18 | 5.55E−10 | 6.20E+05 | 3.44E−04 |
| 1524/1525 | 10 | <1 nM | Yes | Yes | Yes | −0.391 | 9.11 | 8.11E−10 | 1.71E+06 | 1.39E−03 |
| 1526/1527 | 15 | <1 nM | Yes | Yes | Yes | −0.388 | 8.99 | 4.30E−10 | 4.35E+05 | 1.87E−04 |
| 1542/1135 | 11 | <1 nM | Yes | Yes | Yes | −0.411 | 9.2 | 4.63E−10 | 2.16E+05 | 1.00E−04 |

Two anti-OX40 antibodies were synthesised solely for use as reference antibodies for the purposes of comparison in these studies. They are designated herein as “72” or “72/76”, and “74” or “74/78”, respectively.

Measurement of Kinetic Constants by Surface Plasmon Resonance

Human OX40 (R&D systems, #3358_OX) was immobilized to the Biacore™ sensor chip, CM5, using conventional amine coupling. The tested antibodies and controls (serially diluted 1/3 or 1/2 100-2 nM) were analyzed for binding in HBS-P (GE, # BR-1003-68) at a flow rate of 30 μl/ml. The association was followed for 3 minutes and the dissociation for 20 minutes. Regeneration was performed twice using 50 mM NaOH for 60 seconds. The kinetic parameters and the affinity constants were calculated using 1:1 Langmuir model with drifting baseline. The tested antibodies were overall in the subnanomolar-nanomolar range with varying on and off rates (FIG. 6 and Table 3.1). Most of the antibodies had affinities <5 nM. The kinetic parameters and the affinity constants were calculated using BIAevaluation 4.1 software.

Measurement by ELISA of Binding to Human and Murine OX40, and to CD137 and CD40 by ELISA

ELISA plates were coated with human OX40 (R&D Systems, 3388-OX), CD40 (Ancell), or CD137 (R&D Systems) at 0.1 or 0.5 μg/ml. The ELISA plates were washed with PBST and then blocked with PBST+2% BSA for 1 h at room temperature and then washed again with PBST. The antibodies were added in dilution series to the ELISA plates for 1 h at room temperature and then washed with PBST. Binding was detected using goat anti human kappa light chain HRP, incubated for 1 h at room temperature. SuperSignal Pico Luminescent was used as substrate and luminescence was measured using Fluostar Optima.

All the tested OX40 antibodies bound to human OX40 and displayed EC50 value below 1 nM. The antibodies did not bind to murine OX40 or to the other TNFR super family members tested (data not shown).

Measurement of Binding to Human OX40 Over-Expressed on CHO Cells

The extracellular part of human OX40 was fused to the transmembrane and intracellular part of hCD40 and cloned into pcDNA3.0. The vector was subsequently stably transfected into CHO cells. Expression of OX40 was confirmed by incubating with commercial OX40 antibody (huOX40, BD Biosciences) for 30 min at 4° C. and then detected with a-huIgG-PE (Jackson lmmunoresearch) 30 min 4° C. For the assay, the transfected cells were incubated with the test antibodies and controls for 30 min at 4° C. and then detected with a-huIgG-PE (Jackson lmmunoresearch) 30 min 4° C. Cells were analyzed by flow cytometry with FACS Verse (BD Biosciences).

All clones bound to hOX40 overexpressed on CHO cells in a dose dependent manner (FIG. 7).

### Example 4—Sequence Analysis of OX40 Antibodies

The CDR sequences of both the heavy and light chain variable regions were analysed for each antibody. Table 4.1 illustrates the analysis as conducted for the VH CDR3 sequences. Positions in Table 4.1 are defined according to IMGT numbering system. The following patterns were identified.

The VH regions all comprise:

(a) a heavy chain CDR1 sequence which is 8 amino acids in length and comprises the consensus sequence: “G, F, T, F, G/Y/S, G/Y/S, Y/S, Y/S/A”;

(b) a heavy chain CDR2 sequence which is 8 amino acids in length and comprises the consensus sequence: “I, G/Y/S/T, G/S/Y, S/Y, G/S/Y, G/S/Y, G/S/Y, T”; and

(c) a heavy chain CDR3 sequence which is 9 to 17 amino acids in length and which comprises the consensus sequence of: “A, R, G/Y/S/H, G/Y/F/V/D, G/Y/P/F, −/H/S, −/N/D/H, −/Y/G, −/Y, −/Y, −/W/AN, −/A/Y, −/D/A/Y/G/H/N, Y/S/W/A/T, L/M/I/F, D, Y”.

The VL regions all comprise:

(a) a light chain CDR1 sequence which consists of the sequence: “Q, S, I, S, S, Y”;

(b) a light chain CDR2 sequence which consists of the sequence: “A, A, S”;

(c) a light chain CDR3 sequence which is 8 to 10 amino acids in length and comprises the consensus sequence: “Q,Q, S/Y/G, −/Y/H/G, −/S/Y/G/D/W, S/Y/G/D, S/Y/G/T, P/L, Y/S/H/L/F, T”.

Within the consensus sequence for the heavy chain CDR3, two sub-families were identified. Each antibody in the first sub-family comprises a VH CDR3 sequence of 10 amino acids in length which comprises the consensus sequence “A, R, Y/H, D, Y, A/Y/G, S/W/A, M/L, D, Y”. Antibodies in this family are referred to as family Z and are identified as such in Table 4.1. Each antibody in the second sub-family comprises a VH CDR3 sequence of 11 amino acids in length which comprises the consensus sequence “A, R, G/Y, V/F/Y, P, H, G/Y/H, Y, F/I, D, Y”. Antibodies in this family are referred to as family P and are identified as such in Table 4.1. Antibodies of family Z or P are preferred. Antibodies having a VH sequence in family P typically also include a VL sequence with a CDR3 sequence of “Q, Q, S, Y, S, T, P, Y, T”, a CDR1 sequence “Q,S,I,S,S,Y” and a CDR2 sequence of “A,A,S”. Accordingly, antibodies with a VL region comprising these three CDR sequences are preferred.

TABLE 4.1

| | | | | | | | | | | | | | | | | | | CDRH3 | |
|---|---|---|---|---|---|---|---|---|---|---|---|---|---|---|---|---|---|---|---|
| VH | 105 | 106 | 107 | 108 | 109 | 110 | 111 | 111.1 | 111.2 | 112.2 | 112.1 | 112 | 113 | 114 | 115 | 116 | 117 | LENGTH | FAMILY |
| 1482 | A | R | G | Y | G | | | | | | | | | Y | L | D | Y | 9 | |
| 1166 | A | R | Y | D | Y | | | | | | | | A | S | M | D | Y | 10 | Z |
| 1170 | A | R | Y | D | Y | | | | | | | | Y | W | M | D | Y | 10 | Z |
| 1524 | A | R | H | D | Y | | | | | | | | G | A | L | D | Y | 10 | Z |
| 1164 | A | R | G | V | P | H | | | | | | | G | Y | F | D | Y | 11 | P |
| 1168 | A | R | Y | F | P | H | | | | | | | Y | Y | F | D | Y | 11 | P |
| 1490 | A | R | Y | Y | P | H | | | | | | | H | Y | I | D | Y | 11 | P |
| 1542 | A | R | G | Y | P | H | | | | | | | H | Y | F | D | Y | 11 | P |
| 1514 | A | R | S | G | Y | S | N | | | | W | A | N | S | F | D | Y | 14 | |
| 1526 | A | R | Y | Y | F | H | D | Y | | | A | A | Y | S | L | D | Y | 15 | |
| 1520 | A | R | Y | Y | Y | S | H | G | Y | Y | V | Y | G | T | L | D | Y | 17 | |

### Example 5—Domain Mapping of OX40 Antibodies

The extracellular part of OX40 consists of four domains, each of which can be subdivided into two modules. Genes of OX40 human/mouse chimeras were synthesized using standard laboratory techniques. The different chimeras were designed by exchanging domains or modules of the human OX40 with corresponding mouse OX40. The chimeras were designed based on evaluation of the human and mouse sequences and 3D investigation of human OX40. The synthesized genes were assigned project specific ID numbers (see Table 5.1). The constructs were cloned into pcDNA3.1 vector (Invitrogen).

The mouse/human chimeras were transiently transfected into FreeStyle 293-F cells (Invitrogen), incubated 48 hours in FreeStyle 293 expression medium (Invitrogen) 37 C, 8% CO₂, 135 rpm. The transfected cells were incubated with human OX40 antibodies, human OX40L (hOX40L, RnD Systems), mouse OX40L (mOX40L, RnD Systems) and controls for 30 min 4° C. and then detected with a-huIgG-PE (Jackson lmmunoresearch) 30 min 4° C. Cells were analyzed with FACS Verse (BD Biosciences). Binding to the different chimeric constructs were calculated as relative (mean fluorescence intensity) MFI compared to the binding of the isotype control. Results are shown in Table 5.2.

None of the human OX40 antibodies tested bind to murine OX40. Accordingly, if a given antibody does not bind to a particular chimera, this indicates that the antibody is specific for one of the domains which has been replaced with a murine domain in that chimera.

TABLE 5.1
Identity of chimeric constructs

| ID | |
|---|---|
| construct | Description of coding region of the chimeric DNA constructs |
| 1544 | Human OX40 with mouse domains 1A, 1B and 2A (aa 30-81) |
| 1545 | Human OX40 with mouse domains 1B, 2A and 2B |
| | (aa 43-107) |
| 1546 | Human OX40 with mouse domains 2A, 2B and module 3 |
| | (aa 66-126) |
| 1547 | Human OX40 with mouse domain 2B, module 3 and |
| | domain 4A (aa 83-141) |
| 1548 | Human OX40 with mouse module 3 and domains 4A |
| | and 4B (aa 108-167) |
| 1549 | Human OX40 with mouse domains 1A and 4B and region |
| | non-annotated extracellular region (aa 30-65 and |
| | aa 127-214) |
| 84 | Construct containing the full length OX40 sequence |
| 57 | Empty vector (negative control) |

At least four separate binding patterns were identified.

Pattern A:

Antibodies 1170/1171, 1524/1525, and 1526/1527 display a similar binding pattern and depend on residues in the same domains for binding. Amino acid residues critical for binding are likely located in module B in domain 2, and in module A of domain 2. The majority of the antibodies with CDRH3 family “Z” bind according to pattern A (1166/1167 being the exception), indicating that antibodies with this type of CDRH3 are predisposed to bind this epitope.

Pattern B:

Antibodies 1168/1135, 1542/1135, 1520/1135, 1490/1135, 1482/1483 and 1164/1135 display a similar binding pattern and depend mainly on residues located in Domain 3 for binding. All antibodies with CDRH3 family “P” binds with this pattern, demonstrating that the similarity in CDRH3 sequence reveals a common binding epitope.

Pattern C:

Antibody 1166/1167 has a unique binding pattern and likely depends on residues located in module A and module B in domain 2 for binding. However, both modules must be exchanged simultaneously to abolish binding, suggesting a structurally complex epitope.

Pattern D:

Antibody 1514/1515 displays a unique binding profile and likely depends mostly on amino acids located in module B in domain 2 for binding.

Reference antibody 72 binds according to pattern B. The binding pattern of the human OX40 ligand is similar to pattern C.

TABLE 5.2
Results from domain mapping Experiment

| Chimeric | Antibody | | | | | | | | | | | |
|---|---|---|---|---|---|---|---|---|---|---|---|---|
| OX40 | 1490/ | 1170/ | 1524/ | 1526/ | 1482/ | 1514/ | 1164/ | 1168/ | 1520/ | 1542/ | 1166/ | polyclonal |
| construct | 1135 | 1171 | 1525 | 1527 | 1483 | 1515 | 1135 | 1135 | 1135 | 1135 | 1167 | anibody |
| 1544 | 4.2 | 2.0 | 1.5 | 1.1 | 8.4 | 11.6 | 14.0 | 13.2 | 9.4 | 7.1 | 14.2 | 50.9 |
| 1545 | 28.9 | 1.0 | 0.9 | 1.3 | 44.4 | 1.2 | 37.3 | 46.6 | 32.6 | 40.2 | 1.0 | 58.6 |
| 1546 | 1.0 | 0.8 | 0.9 | 0.9 | 0.9 | 0.9 | 0.9 | 0.9 | 0.9 | 0.9 | 0.8 | 30.0 |
| 1547 | 1.1 | 2.3 | 1.0 | 1.0 | 1.0 | 1.1 | 1.0 | 1.0 | 0.9 | 1.0 | 15.2 | 43.9 |
| 1548 | 1.1 | 20.3 | 15.4 | 12.7 | 1.1 | 17.2 | 1.0 | 1.0 | 1.1 | 1.2 | 15.8 | 31.7 |
| 1549 | 5.9 | 12.1 | 11.2 | 8.5 | 8.7 | 10.6 | 14.1 | 15.1 | 8.5 | 13.0 | 11.3 | 26.3 |
| 84 | 14.2 | 33.4 | 31.4 | 21.4 | 24.9 | 25.9 | 27.5 | 29.6 | 25.4 | 29.4 | 27.6 | 53.2 |
| 57 | 1.0 | 1.0 | 1.0 | 1.0 | 1.0 | 1.1 | 1.0 | 0.9 | 1.0 | 1.1 | 1.0 | 1.2 |

### Example 6—Cross-Reactivity with Macaca mulatta

The extracellular part of OX40 from Macaca mulatta was fused to the transmembrane and intracellular part of hCD40 and cloned into pcDNA3.0. The vector was subsequently stably transfected into HEK cells (macOX40-HEK).

Expression of OX40 was confirmed by incubating with commercial OX40 antibody (huOX40, BD Biosciences) for 30 min at 4° C. and then detected with a-huIgG-PE (Jackson Immunoresearch) 30 min 4° C. For the assay, the transfected cells were incubated with the test antibodies and controls for 30 min at 4° C. and then detected with a-huIgG-PE (Jackson lmmunoresearch) 30 min 4° C. Cells were analyzed by flow cytometry with FACS Verse (BD Biosciences).

As shown in Table 6.1 below, tested antibodies bind to Macaca mulatta OX40 with EC50 values comparable to those achieved for human OX40, suggesting that Macaca mulatta will be suitable for use in toxicology studies.

Macaca mulatta is genetically very similar to Macaca fascicularis (cynomolgus monkey) making it very likely that cynomolgus monkey is also a suitable species for toxicology studies.

TABLE 6.1
Binding to human and monkey OX40 (95% confidence intervals)

| | Binding to M mulatta | |
|---|---|---|
| | OX40, EC50 | Binding to human OX40, EC50 |
| OX40 antibody | (μg/ml) | (μg/ml) |
| 1166/1167 | 0.1595 to 0.2425 | 0.1415 to 0.2834 |
| 1168/1135 | 0.09054 to 0.1939 | 0.06360 to 0.1308 |
| 1482/1483 | 0.1565 to 0.3120 | 0.08196 to 0.1822 |
| 1520/1135 | 0.1632 to 0.3587 | 0.09247 to 0.2749 |
| 1526/1527 | 0.2921 to 0.5888 | 0.1715 to 0.4292 |
| 1542/1135 | 0.7221 to 1.414 | 0.3223 to 0.5525 |

### Example 7—Agonistic Activity in a Human T Cell Assay

Human T cells were obtained by negative T cell selection kit from Miltenyi from PBMC from leucocyte filters obtained from the blood bank (Lund University Hospital). The OX40 antibodies were coated to the surface of a 96 well culture plate (Corning Costar U-shaped plates (#3799) and cultured with a combination of immobilized anti-CD3 antibody (UCHT1), at 3 μg/ml, and soluble anti-CD28 antibody (CD28.2), at 5 μg/ml. Anti-CD3 was pre-coated overnight at 4° C. On the following day, after one wash with PBS, the OX40 antibodies were coated 1-2 h at 37° C. After 72 h incubation in a moisture chamber at 37° C., 5% CO₂ the IL-2 levels in the supernatant were measured.

The ability of the antibodies to stimulate human T cells to produce IL-2 was compared with the reference antibody 74 and the relative activity is displayed in FIG. 8. The majority of the antibodies provided T cell activation levels that were comparable with the reference antibody. A number of antibodies provided higher levels of T cell activation.

Bispecific Molecules

In the following Examples, tested bispecific molecules are referred to by number, e.g. 1164/1141. This means that the molecule comprises the amino acid sequences of the respective VH and VL regions shown in Tables B and D. For example, 1164/1141 comprises the heavy chain VH region sequence 1164 shown in Table B (SEQ ID NO: 99), and the bispecific chain number 1141 shown in Table D (SEQ ID NO: 129). The specified VH region sequence of a given molecule is typically provided linked (as part of a single contiguous polypeptide chain) to the IgG1 heavy chain constant region sequence of SEQ ID NO: 135. This sequence is typically present at the C terminal end of a specified VH region sequence of Table B.

### Example 8—Affinity of Exemplary Bispecific Molecules for Binding to Single Targets

Measurement of Kinetic Constants by Surface Plasmon Resonance

Human OX40 (R&D systems, #3358_OX) was immobilized to the Biacore™ sensor chip, CM5, using conventional amine coupling. The tested antibodies and controls (serially diluted 1/3 or 1/2 100-2 nM) were analyzed for binding in HBS-P (GE, # BR-1003-68) at a flow rate of 30 μl/ml. The association was followed for 3 minutes and the dissociation for 20 minutes. Regeneration was performed twice using 50 mM NaOH for 30 seconds. The kinetic parameters and the affinity constants were calculated using 1:1 Langmuir model with drifting baseline. The tested molecules had varying on and off rates with generally lower affinity for OX40 than the corresponding monomeric antibodies, but were still in the nanomolar range (Table 8.1).

TABLE 8.1

| BsAb | ka (1/Ms) | kd (1/s) | KD (nM) |
|---|---|---|---|
| 1164/1141 | 8.87E+04 | 1.72E−04 | 1.94 |
| 1168/1141 | 2.84E+05 | 3.05E−04 | 1.07 |
| 1166/1261 | 7.04E+04 | 1.12E−04 | 1.59 |
| 1170/1263 | 5.18E+05 | 6.39E−04 | 1.23 |

Measurement by ELISA

ELISA plates were coated with human with CTLA-4 (BMS, Orencia) or human OX40 (R&D Systems, 3388-OX) at 0.4 or 0.5 μg/ml, respectively. The ELISA plates were washed with PBST and then blocked with PBST+2% BSA for 1 h at room temperature and then washed again with PBST. The bispecific molecules were added in dilution series to the plates and incubated for 1 h at room temperature. The ELISA plates were washed, and binding was detected using goat anti human kappa light chain HRP for 1 h at room temperature. SuperSignal Pico Luminescent was used as substrate and luminescence was measured using Fluostar Optima.

All the tested bispecific molecules bound to both targets and the EC50 values are in the range that would be expected based on their affinity as monospecific antibodies (FIG. 9).

### Example 9—Dual Binding to Both Targets of Exemplary Bispecific Molecules

Measurement by Surface Plasmon Resonance

Human OX40 (R&D systems, #3358_OX) was immobilized to the Biacore™ sensor chip, CM5, using conventional amine coupling. The tested bispecific molecules (0.5 μM or 0.25 μM) and controls were run over the chip at a flow rate of 30 μl/ml. The association was followed for 3 minutes and the dissociation for 3 minutes. CTLA4-Fc (BMS, Orencia) was then injected and association followed for 3 minutes and the dissociation for 3 minutes. As a control a blank PBS was injected instead of CTLA4.

All the tested bispecific molecules bound to both targets simultaneously, as is shown in FIG. 10).

Measurement by ELISA

ELISA plates were coated with OX40-Fc (R&D systems, #3358_OX) (0.4 μg/ml) over night at 4° C. The ELISA plates were washed with PBST and then blocked with PBST+2% BSA for 1 h at room temperature and then washed again with PBST. Bispecific molecules were added in dilutions to the plates and incubated for 1 h at room temperature. The ELISA plates were washed and biotinylated CTLA-4 (1 μg/ml) was added and incubated on the plates at room temperature. The plates were washed and HRP-labelled streptavidin was used for detection of binding. SuperSignal Pico Luminescent was used as substrate and luminescence was measured using Fluostar Optima.

Binding to both targets was confirmed for all tested bispecific molecules. As is shown in FIG. 11, the tested bispecific molecules could be detected at a concentration 0.1 nM, which corresponds to 0.015 μg/ml. The relative values in the assay correspond well to the affinities measured by surface plasmon resonance.

### Example 10—Agonistic Activity of Exemplary Bispecific Molecules in a Human CD4 T Cell Assay

Human CD4 T cells were isolated by negative CD4 T cell selection (Miltenyi, human CD4+ T cell Isolation Kit 130-096-533) of PBMC from leucocyte filters obtained from the blood bank (Lund University Hospital). CTLA-4 (Orencia, 2.5 μg/ml) and anti-CD3 (UCHT-1, 1 ug/ml) was coated to the surface of a 96-well culture plate (non-tissue cultured treated, U-shaped 96-well plates (Nunc, VWR #738-0147) over night at 4° C. By coating with both CTLA-4 and CD3, the assay provides an experimental model of a tumour microenvironment with over-expressed CTLA-4. CTLA-4 was omitted from some wells as a control.

Bispecific molecules to be tested were added soluble in a serial dilution to the wells and compared at the same molar concentrations with controls. Two different controls were used for each bispecific molecule tested. The first control is a bispecific molecule designated 1756/1757 (an isotype control antibody fused to the 1040 CTLA4 binding region=binds CTLA4 but not OX40). The second control is a mixture of the bispecific 1756/1757 control and the monospecific OX40 antibody, which corresponds to the tested bispecific molecule. After 72 h of incubation in a moisture chamber at 37° C., 5% 002, IL-2 levels were measured in the supernatant.

As shown in FIG. 12, there is a dose-dependent effect of the bispecific molecules, which induce an increase in human T cell activation (measured by an increase in IL-2 production) when cultured in plates coated with CTLA-4. The controls do not. FIG. 13 shows the results of the same assay when conducted at a fixed concentration for the bispecific antibodies and controls (1.5 nM) in the presence or absence of CTLA-4. The increase in T cell activation is not seen in the absence of CTLA-4. The fold change in IL-2 levels induced by each bispecific molecule compared to the corresponding combination of monospecific molecules is shown in Table 10.1. The mean value of IL-2 produced (pg/ml) for each bispecific molecule or control is shown in Table 10.2.

Since this assay represents an experimental model of a tumour microenvironment with over-expressed CTLA-4, the results suggest that the tested bispecific molecules can be expected to have a greater effect than monospecific antibodies in such a microenvironment.

TABLE 10.1
Fold change in IL-2 level induced by bispecific
molecule compared to the corresponding

| 1164/1141 | 1166/1261 | 1168/1141 | 1170/1141 | 1514/1581 | 1520/1141 |
|---|---|---|---|---|---|
| 7.6 | 7.8 | 7.2 | 4.7 | 5.5 | 1.7 |

combination of the monospecific molecules at 1.5 nM

TABLE 10.2
Mean IL-2 level (pg/ml) induced at 1.5 nM bispecific antibody or control

| | 1164/ | 1166/ | 1164/1135 + | 1166/1167 + | 1168/ | 1170/ | 1168/1135 + | 1170/1171 + | 1514/ |
|---|---|---|---|---|---|---|---|---|---|
| | 1141 | 1261 | 1756/1757 | 1756/1757 | 1141 | 1141 | 1756/1757 | 1756/1757 | 1581 |
| Mean | 5024 | 4292 | 665 | 550 | 2681 | 2575 | 371 | 552 | 5109 |
| SD | 1058 | 1333 | 273 | 456 | 954 | 992 | 162 | 285 | 2600 |

| | 1520/ | 1514/1515 + | 1520/1135 + | 1526/ | 1542/ | 1526/1543 + | 1542/1543 + | 1756/ |
|---|---|---|---|---|---|---|---|---|
| | 1141 | 1756/1757 | 1756/1757 | 1585 | 1141 | 1756/1757 | 1756/1757 | 1757 |
| Mean | 1303 | 927 | 774 | 3200 | 2671 | 746 | 697 | 1047 |
| SD | 812 | 653 | 451 | 1350 | 1016 | 346 | 418 | 601 |

### Example 11—Stability of Exemplary Bispecific Molecules

The melting point of the antibodies was analyzed by differential scanning fluorimetry (DSF). Antibody samples in PBS were mixed with SYPRO Orange which was diluted 1000-fold. Thermal scanning between 25 and 95° C. was performed in a real-time PCR machine with measurements made each degree. A reference antibody 250/251 was used for comparison and the difference in melting temperature Tm (ΔTₘ) relative to the reference was determined. Tₘ differences of more than 1.1° C. compared to the reference are considered statistically significant. As shown in Table 11.1, all tested bispecific molecules displayed good thermostability with values of 65° C. or above.

TABLE 11.1

| | Melting |
|---|---|
| | temperature |
| Antibody | (° C.) |
| 1168/1141 | 65.6 |
| 1164/1141 | 65.5 |
| 1160/1259 | 68.5 |
| 1166/1261 | 67.8 |
| 1170/1263 | 66.4 |
| 1514/1581 | 65.3 |
| 1520/1141 | 65.0 |
| 1526/1585 | 67.6 |
| 1542/1141 | 66.3 |

### Example 12—Synergism in Induction of ADCC by Exemplary Bispecific Antibodies Targeting CTLA4 and OX40 (Compared to Effect of Monospecific Antibodies, Alone or in Combination)

Materials and Methods

Assessment of Antibody-Dependent Cell-Mediated Cytotoxicity (ADCC)

Jurkat cells engineered to stably express FcγRIIIa receptor (V158 variant) and an NFAT response element driving expression of firefly luciferase (Promega Corporation) were used as effector cells in the assessment of ADCC. Antibodies were titrated in duplicate wells in a 96-well opaque luminescence plate, and effector cells and target cells expressing both OX40 and CTLA4 were added in a ratio of 5:1. After 6 h incubation in a 37° C., 95% O₂ humidified incubator, luciferase assay substrate (Promega Corporation) was added to all wells including medium control wells (for blank subtraction), and luminescence was detected on a FLUOstar Optima microplate reader (BMG LabTech). Fold-induced ADCC was calculated as: (target lysis−blank)/(spontaneous lysis−blank). Top values were calculated based on log(agonist) vs. response (three parameters) curve fit using Prism 6.0 (Graphpad, La Jolla, Calif., USA).

Antibodies
“1166/1167”=monospecific OX40 antibody
“Control IgG with CTLA-4 binding part”=monospecific CTLA4 binding domain fused to an IgG protein
“1166/1261”=exemplary bispecific antibody targeting OX40 and CTLA4 (containing the identical OX40 and CTLA-4 binding part as the monospecific binders described above).
“Ctrl IgG”=negative isotype control

Results

Exemplary Bispecific Antibody 1166/1261 Exhibits Superior Induction of ADCC

As shown in FIG. 15, detectable levels of ADCC were induced by all tested components. The negative isotype control did not induce any ADCC (data not shown). Most notably, the bispecific 1166/1261 antibody induced ˜123-fold ADCC compared to control. The monospecific OX40 antibody (1166/1167) induced ˜29-fold and the monospecific CTLA-4 binding domain (62/376) induced ˜10-fold ADCC, whereas the mixture of the two monospecific components (1166/1167+62/376) induced ˜31-fold ADCC.

There is thus an unexpected and marked synergy obtained by the bispecific molecule binding to OX40 and CTLA-4.

### Example 13—Bispecific Antibodies Targeting OX40 and CTLA4—Specific Binding to Cells Expressing Both OX40 and CTLA4

### Background

The aim of this study was to determine the binding efficacy and EC50 of 1166/1261 and the corresponding monospecific binding entities to cells expressing both OX40 and CTLA4 using flow cytometry. The bispecific antibody is designed to bind both OX40 and CTLA4 simultaneously. For this purpose, we used transfected CHO cells with a stable expression of our targets. CHO P4 cells have a high expression level of both OX40 and CTLA4.

Methods and Results

Double-transfected CHO cells expressing both OX40 and CTLA4 were originally sorted by FACS (Beckton Dickinson) into a cell pool expressing high levels of both targets (denoted CHO P4). Target expression was kept stable by culturing the cells under selection pressure of geneticine and zeocine. Untransfected CHO wild-type cells were used as controls.

Cells were stained with decreasing concentrations of 1166/1261 (an exemplary bispecific antibody targeting OX40 and CTLA4), or the two monospecific binders 1166/1167 (OX40 specific monoclonal antibody) and Control IgG with CTLA-4 binding part (monospecific CTLA4 binding IgG fusion protein) (200 nM-0.0034 nM), followed by PE-conjugated anti-human IgG. Fluorescence was detected using a FACSverse instrument, and the acquisition was analysed using FlowJo software. The median fluorescent intensity (MFI) was determined for each staining.

Binding efficacy curves for CHO P4 are presented in FIG. 16 (one representative experiment out of three). 1166/1261 binds to cells with high expression of OX40 and CTLA4 better than 1166/1167 or the Control IgG with a CTLA-4 binding entity. This is probably an additive effect of 1166/1261 being able to bind two targets simultaneously.

### Example 14—Bispecific Antibodies Targeting OX40 and CTLA4—Dual Binding of Cells Expressing OX40 and CTLA4 Measured by Flow Cytometry

Aim

Measure simultaneous binding by 1166/1261 to both OX40 and CTLA4 over-expressed on cells by measuring the number of aggregated cells using flow cytometry.

Materials and Methods

CHO-OX40 cells and HEK-CTLA4 cells were intracellularly stained with the fluorescent dyes PKH-67 (green fluorescent dye) respectively PKH-26 (red fluorescent dye) (Sigma-Aldrich). After verifying homogenously stained cell population, the cells were mixed and incubated with either 1166/1261 (an exemplary bispecific antibody targeting OX40 and CTLA4) or a combination of the two monoclonal antibodies 1166/1167 (a monospecific anti-OX40 antibody) and a control IgG comprising a CTLA4-binding domain. After staining the cells were immediately fixed and the number of aggregated, double-positive cells were quantified using FACS-verse (BD biosciences). Data analyses and non-linear regression was performed using Graph Pad Prism v6.

### Results and Conclusions

Exemplary bispecific antibody 1166/1261 increases the number of aggregated cells with increasing concentration (FIG. 17) (one representative experiment out of two).

### Example 15—Bispecific Antibodies Targeting OX40 and CTLA4—Pharmacokinetics in Mice

Material and Methods

Antibodies
1166/1261 (an exemplary bispecific antibody targeting OX40 and CTLA4)
1166/1167 (a monospecific control antibody targeting OX40)

In Vivo Studies

Female C57BL/6 (7-8w) mice from Taconic's Denmark were used in the experiments. All experiments were done by approval of Malmö/Lund ethical committee.

The mice were injected intraperitoneally with 100 μg of each antibody and blood was drawn either via vena saphena or at termination via vena cava into heparinized tubes after 0 h, 1 h, 4 h, 8 h, 24 h, 72 h and after 1 week. 3 mice were used for each time-point. Blood was spun at 2500 rpm for 30 min and plasma was frozen to −80 C° for further analysis.

Assays for Determination of 1166/1261 and 1166/1167 Levels in Plasma

Two different assays were used. A single target ELISA (ELISA1) and a dual ELISA (ELISA2). Briefly the assays consisted of the following steps. White high-binding flat-bottom, LIA plates (Greiner Bio-One, Austria) were coated over night with 0.8 μg/mL humanOX40-Fc (RnD Systems, MN, USA). After washing with Washing buffer (phosphatase buffer saline supplemented with 0.05% Tween 20 (PBST), Medicago, Sweden) the wells were blocked using PBST with 2% bovine serum albumin (BSA) (Merck, Germany) for 1 hour at ambient room temperature (ART) with shaking and washed again before plasma samples diluted 1:200 and 1:5000 in assay buffer (PBST+0.5% BSA) together with calibration curve samples (1166/1261, conc. 6-0.0012 μg/mL) were added. After incubation at ART for 1 h with shaking and subsequent washing, secondary reagent was added, consisting of either human anti-kappa-antibody horse radish peroxidase conjugated (HRP) (AbD Serotec, UK) for the single target ELISA or biotinylated human CTLA-4-Fc (Orencia) at 1 μg/mL followed by streptavidin-HRP (Thermo Fisher Scientific, MN, USA) according to the manufacturer's instructions for the dual ELISA. Signal was obtained using HRP substrate SuperSignal Pico Luminescence (Thermo Fisher Scientific). Luminescence measurements were collected after 10 minutes incubation in darkness with shaking using a Flurostar Optima (MBG Labtech, Germany). The data were analyzed by using GraphPad Prism program.

Results

Samples collected at the different time points after injection with 1166/1261 and 1166/1167 were analyzed with only single target ELISA or single target and dual ELISA for determination of the plasma levels of 1166/1261 and 1166/1167 respectively. The results show that the levels of 1166/1261 and 1166/1167 in plasma increased around the first 4 hours after peritoneal injection and then reduced (FIG. 18 upper panel). Detectable levels of both 1166/1261 and 1166/1167 are present in plasma after one week (FIG. 18 middle and lower panels).

The levels of 1166/1261 in plasma are similar to the levels obtained for the monoclonal antibody 1166/1167 indicating that 1166/1261 exhibits a good half-life in vivo, comparable to that of an equivalent monospecific anti-OX40 antibody.

### REFERENCES

Hemerle T., Wulhfard S., Neri D., (2012) A critical evaluation of the tumour-targeting properties of bispecific antibodies based on quantitative biodistribution data. Protein Engineering and Design, 25, pp 851-854.

### Example 16—Bispecific Antibodies Targeting OX40 and CTLA4—In Vivo Anti-Tumour Effect in HT-29 Colon Cancer Model

### Summary

The anti-tumour effect of 1166/1261 (an exemplary bispecific antibody targeting OX40 and CTLA4) was investigated using hPBMC humanized immunodeficient mice and subcutaneous tumour models of HT-29 colon carcinoma.

1166-1261 demonstrated statistically significant tumour volume inhibition.

Material and Methods

Female SCID-Beige mice (6-9w) from Taconic's Denmark were used in the experiments. All experiments were done by approval of Malmö/Lund ethical committee.

HT-29 colon cancer cells were obtained from ATCC and cultivated according to ATCC recommendations. The HT-29 cell line growing in log phase was injected subcutaneously (4×10⁶ cells in 100-200 μL at day 0 (DO)). Human PBMC (7×10⁶ in 200 μL) isolated from leukocyte concentrates was injected intraperitoneally at the same day. Intraperitoneal treatments (667 pmol) were done on days 6, 13, and 20.

Leukocyte concentrates were obtained from Lund University Hospital.

Tumour was measured with a calliper in width, length and height of which the tumour volume was calculated (w/2×l/2×h/2×pi×(4/3)). The animals were terminated before the tumour volume reached 2 cm³, at wounding, or affected health of the mice.

The data were analyzed by Mann-Whitney test using the GraphPad Prism program. Responder donors were considered those donors that were responsive to the reference antibody 1874. Minimum of 10% average tumour inhibition during the exponential tumour growth period was considered as a response.

Results

Pooled data from mice engrafted with responder donors (4 donors from two separate experiments) demonstrated statistically significant anti-tumour efficacy at days 12-16 in form of tumour growth inhibition when treated with the 1166/1261 antibody (p=0.0469 to p=0.0074, Mann-Whitney non-parametric, 2-tail) in comparison to the vehicle group (ZZ). The percentage of tumour volume inhibition ranged from 22-36% with 1166/1261 between days 10 and 21 (see FIG. 19 and Table 31.1).

In conclusion, the anti-tumour effect of 1166/1261 was investigated using hPBMC humanized immunodeficient mice and subcutaneous tumour models of HT-29 colon carcinoma. 1166/1261 demonstrated statistically significant tumour volume inhibition.

TABLE 31.1
Statistical analysis and percent tumour inhibition

| Day | | |
|---|---|---|
| after tumour | Tumour growth inhibition (tumour | p-value |
| inoculation | volume) compared to vehicle (%) | (Mann-Whitney 2-tail) |
| D10 | 22.8 | 0.1298 |
| D12 | 35.4 | 0.0315 |
| D14 | 35.9 | 0.0074 |
| D16 | 31.5 | 0.0469 |
| D19 | 30.8 | 0.1059 |
| D21 | 22.1 | 0.1067 |

### Example 17—Bispecific Antibodies Targeting OX40 and CTLA4—In Vivo Anti-Tumour Effect in Raji Lymphoma Model

### Summary

The anti-tumour effect of 1166/1261 (an exemplary bispecific antibody targeting OX40 and CTLA4) was investigated using hPBMC humanized immunodeficient mice and subcutaneous tumour models of Raji B-cell lymphoma.

1166/1261 demonstrated statistically significant tumour volume inhibition.

Material and Methods

Female SCID-Beige mice (6-9w) from Taconic's Denmark were used in the experiments. All experiments were done by approval of Malmö/Lund ethical committee.

Raji B-cell lymphoma was obtained from ATCC and cultivated according to ATCC recommendations. The Raji cell line growing in log phase was injected subcutaneously (10×10⁶ cells) together with human PBMC (10×10⁶ in 200 μL), isolated from buffy coats. Intraperitoneal treatments (667 pmol) were done on days 0, 7, and 14.

Buffy coats were obtained from Kalmar University Hospital.

Tumour size was measured with a caliper in width, length and height of which the tumour volume calculated (w/2×l/2×h/2×pi×(4/3)). The animals were terminated before the tumour volume reached 2 cm³, at wounding, or affected health of the mice.

The data were analyzed by Mann-Whitney test using the GraphPad Prism program. Responder donors were considered those donors that were responsive to the reference antibody 1874. Minimum of 10% average tumour inhibition during the exponential tumour growth period was considered as a response.

### Results and Conclusions

Pooled data from experimental groups with responding donors, the bispecific 1166/1261 antibody demonstrated statistically significant anti-tumour efficacy at days 14 and 21 (p=0.0068 and p=0.0288, Mann-Whitney, 2-tail) in comparison to the vehicle (Table 32.1).

TABLE 32.1
Statistical analysis and percent tumour inhibition

| | Tumour | Tumour | Tumour growth | |
|---|---|---|---|---|
| | volume in | volume in | inhibition (tumour | p-value |
| Day after | vehicle- | animals | volume) | (Mann- |
| tumour | treated | treated with | compared to | Whitney 2- |
| inoculation | animals | 1166/1261 | vehicle (%) | tail) |
| D10 | 14.2 | 13.8 | 6.1 | 0.6842 |
| D12 | 35.7 | 21.5 | 39.9 | 0.0603 |
| D14 | 61.8 | 33.6 | 45.7 | 0.0068 |
| D17 | 105.3 | 76.0 | 27.8 | 0.3527 |
| D19 | 205.1 | 133.3 | 35 | 0.0524 |
| D21 | 314.8 | 187.0 | 40.6 | 0.0288 |
| D24 | 467.5 | 299.9 | 37.2 | 0.054 |
| D26 | 529.7 | 360.1 | 32 | 0.063 |

### Example 18—In Vivo Anti-Tumour Effect in MC38 Colon Cancer Model

### Summary

The anti-tumour effects of OX40-CTLA-4 bispecific antibody 1166/1261 was investigated using transgenic mice for human OX40 and subcutaneous tumour models of MC38 colon carcinoma.

1166/1261 demonstrated a statistically significant effect on CD8/Treg ratio compared to monoclonal counterparts.

Material and Methods

Female transgenic mice for human OX40 (homozygous human OX40 knock-in mouse model, developed by GenOway) were used in the experiments. All experiments were done by approval of Malmö/Lund ethical committee.

MC38 colon cancer was cultivated in RPMI, 10% heat inactivated fetal calf serum, sodium pyruvate, hepes and 2-mercaptoethanol. The MC38 cell line growing in log phase was injected subcutaneously (1×10⁶ cells in 100 μL at day 0 (DO)) and treatments (1.33 mol) were done intraperitoneally on days 10, 13 and 16. Twenty-four hours after last injections, the tumours and spleens were harvested, stained for viability marker, lineage markers CD11b, C19, MHCII, NK1.1, CD45, CD3, CD4, CD8, CD25, Foxp3, Ki-67, and analysed using flow cytometry.

Results

The pharmacodynamics effects of a bispecific OX40-CTLA-4 antibody was investigated in hOX40tg mice using the MC38 colon carcinoma model. Pooled data from two independent experiments demonstrated statistically significant effect on intratumoural CD8/Treg ratio with bispecific antibody compared to both monospecific counterparts (FIG. 20). No changes in the CD8/Treg ratio can be seen in the spleen, with regards to CD8/Treg ration nor the level of Ki-67 expression in the Tregs (Ki-67 is a marker for proliferation). This shows that 1166/1261 induces a significant effect on the Tregs in the tumour that is greater than the sum of the effects obtained by the two monospecific control antibodies without affecting the T cells in the periphery. The effect is thus directed to the tumour microenvironment, which may provide a larger therapeutic window, i.e. a high anti-tumour effect with low systemic toxicity. The relative levels of the different T cell populations are outlined in FIG. 21.

### Example 19—In Vivo Anti-Tumour Effect in MC38 Colon Cancer Model

### Summary

The anti-tumour effects of OX40-CTLA-4 bispecific antibody 1166/1261 was investigated using transgenic mice for human OX40 and subcutaneous tumour models of MC38 colon carcinoma.

1166/1261 demonstrated statistically significant anti-tumour efficacy in form of tumour volume inhibition.

Material and Methods

Female transgenic mice for human OX40 (homozygous human OX40 knock-in mouse model, developed by GenOway) were used in the experiments. All experiments were done by approval of Malmö/Lund ethical committee.

MC38 colon cancer was obtained from Stanford University and cultivated in RPMI, 10% heat inactivated fetal calf serum, sodium pyruvate, hepes and 2-mercaptoethanol. The MC38 cell line growing in log phase was injected subcutaneously (1×10⁶ cells in 100 μL at day 0 (DO)) and treatments (1.33 mol) were done intraperitoneally on days 7, 10 and, 13.

Tumour was measured with a calliper in width, length and height of which the tumour volume was calculated (w/2×l/2×h/2×pi×(4/3)). The animals were terminated before the tumour volume reached 2 cm³, at wounding, or affected health of the mice.

The data were analysed for tumour volume inhibition bispecific antibody compared to monoclonal antibodies and vehicle using the GraphPad Prism and Excel program.

Results

Pooled data from three independent experiments (n=26-28) demonstrated statistically significant effect on tumour volume inhibition and survival.

In conclusion, the anti-tumour effect of bispecific OX40-CTLA-4 antibody was investigated in transgenic mice for human OX40 and using MC38 colon carcinoma model. The bispecific antibody demonstrated statistically significant effects on tumour volume inhibition and increased survival. The anti-tumour effect was stronger in terms of survival and tumour growth inhibition than the monospecific control antibodies (FIG. 22).

### Example 20—In Vivo Anti-Tumour Effect in MB49 Bladder Cancer Model

### Summary

The anti-tumour effect of OX40-CTLA-4 bispecific antibody 1166/1261 was investigated using transgenic mice for human OX40 and subcutaneous tumour models of MB49 bladder carcinoma.

1166/1261 demonstrated statistically significant anti-tumour efficacy in form of tumour volume inhibition and increased survival.

Material and Methods

Female transgenic mice for human OX40 (homozygous human OX40 knock-in mouse model, developed by GenOway) were used in the experiments. All experiments were done by approval of Malmö/Lund ethical committee.

The MB49 cell line growing in log phase was injected subcutaneously (0.25×10⁶ cells) on day 0 and treatments (1.33 μmol) were done intraperitoneally on days 7, 10 and 13.

Tumour volumes were measured with a calliper in width, length and height of which the tumour volume was calculated ((w/2)×(l/2)×(h/2)×pi×(4/3)). The animals were terminated before the tumour volume reached 2 cm³, at wounding, or affected health of the mice. The statistical analysis was done for tumor volume using Mann-Whitney, non-parametric, 2-tail test and for survival Kaplan-Meyer Log-Rank using Graph Pad Prism.

Results

The anti-tumour effect of bispecific OX40-CTLA-4 antibody 1166/1261 was investigated in transgenic mice for human OX40 using MB49 bladder carcinoma model. The bispecific antibody demonstrated statistically significant effects on tumour volume inhibition (FIG. 23A, p=0.0003) and increased survival (FIG. 23B p<0.0001).

### Example 21—Immunological Memory Induced by 1166/1261

### Summary

Immunomodulators are considered to induce long term curative responses against cancer, as they induce immunological memory. To demonstrate such immunological memory, mice in which 1166/1261 had induced complete tumour regression, were re-challenged with the same specific tumour, the MB49 cell line, or with the irrelevant tumour cell line PANC02.

The re-challenge experiment demonstrated that 1166/1261 generated tumour specific immunological memory against MB49 bladder cancer, but not against irrelevant tumour PANC02.

Material and Methods

Female knock-in mice for human OX40 (hOX40tg), generated by genOway, in which 1166/1261 had induced complete tumour regression from MB49 bladder cancer, were re-challenged with MB49 as a specific tumour in a single tumour model or together with irrelevant tumour PANC02 using twin tumour model. Naïve mice were used as tumour growth controls.

MB49 and PANC02 growing in log phase were injected subcutaneously (0.25×10⁶ cells) either as a single tumour or twin tumour, one in each side of the flank. The tumour volume was measured three times a week with a caliper and the tumour volume was calculated using formula (¾×π×(width/2)×(length/2)×(height/2).

Results

The immunological memory was examined in mice showing complete tumour regression after 1166/1261 treatment by re-challenging these mice with the specific tumour MB49 or with an irrelevant tumour. The complete responders demonstrated immunological memory against the specific tumour MB49 as the new tumours failed to grow (FIG. 24A). Naïve mice were used as a positive control for tumour growth. Further, the immunological memory was demonstrated to be tumour specific as the complete responders re-challenged with MB49 and with irrelevant tumour PANC02 in a twin tumour model, demonstrated growth only of the irrelevant tumour but not of the previously encountered MB49 tumour (FIG. 24B). This data demonstrates that 1166/1261 induces tumour specific immunological memory.

### Example 22—In Vivo Anti-Tumour Effects in MB49 Bladder Cancer Model by Assessment of Intratumoural CD8/Treg Ratio

### Summary

In the tumour environment Tregs have a high expression of both OX40 and CTLA-4. OX40-CTLA-4 bispecific antibodies are expected to induce depletion of Tregs in this tumour environment. The ability of 1166/1261 to induce depletion of intratumoural Tregs was investigated using transgenic mice for human OX40 and subcutaneous tumour models of MB49 bladder cancer.

1166/1261 demonstrated statistically significant effects on Treg depletion, activation of effector cells and increased CD8/Treg ratio compared to monoclonal counterparts. This effect was mainly localized to the tumour environment, as no significant effects were observed in the spleen in form of altered CD8/Treg ratio.

Material and Methods

Female homozygotes for human OX40 (hOX40tg) age 7-14w, generated by genOway, France, were used in the experiments. All experiments were done by approval of Malmö/Lund ethical committee.

MB49 bladder cancer growing in log phase was injected subcutaneously (0.25×10⁶ cells) on day 0 and treatments (1.33 μmol) were done intraperitoneally on days 10, 13 and 16. Twenty-four hours after the last injection, the tumours and spleens were harvested, stained for CD45, CD3, CD4, CD8, CD25, Foxp3, as well as for lineage markers (CD19, NK1.1, MHCII) and analysed using flow cytometry. The statistical analysis was done using mann-Whitney, non-parametric, 2-tail, if not otherwise stated, using Graph Pad Prism.

Results

The pharmacodynamics effect of the bispecific OX40-CTLA-4 antibody was investigated in hOX40tg mice using MB49 bladder cancer. The experiments demonstrated that 1166/1261 induced statistically significant effect on intratumoural Treg content (p=0.0087, 2-tail), (FIG. 25A), CD8 numbers in tumours (p=0.047, 1-tail), (FIG. 25B) and CD8/Treg ratio p=0.0043 2-tail) (FIG. 25C). No changes in the CD8/Treg ratio can be seen in the spleen, indicating tumour localization of 1166/1261 (FIG. 25D).

### Example 23—In Vivo Anti-Tumour Effects in MC38 Colon Carcinoma Model by Assessment of Effector Cell Activation

### Summary

In addition to regulatory T cell depletion, part of OX40-CTLA-4 bispecific antibodies mode of action is considered to be activation of effector T cells. These anti-tumour effects of OX40-CTLA-4 bispecific antibody 1166/1261 was investigated using transgenic mice for human OX40 and subcutaneous tumour models of MC38 colon carcinoma.

1166/1261 demonstrated a statistically significant effect on effector CD8 activation in the tumour microenvironment in the form of increased Granzyme B and CD107a expression.

Material and Methods

Female transgenic mice for human OX40 (homozygous human OX40 knock-in mouse model, developed by GenOway) were used in the experiments. All experiments were done by approval of Malmö/Lund ethical committee.

MC38 colon cancer growing in log phase was injected subcutaneously (1×10⁶ cells) on day 0 and treatments (1.33 μmol) were done intraperitoneally on days 10, 13 and 16. Twenty-four hours after last injections, the tumours and spleens were harvested, stained for viability marker, lineage markers (CD11b, C19, MHCII, NK1.1), CD45, CD3, CD4, CD8, Granzyme B and CD107a and analysed using flow cytometry. The data was analyzed using Mann-Whitney, non-parametric, 2-tail test.

Results

The pharmacodynamics effects of a bispecific OX40-CTLA-4 antibody was investigated in hOX40tg mice using MC38 colon carcinoma. The experiments demonstrated that 1166/1261 induced statistically significant activation of the effector CD8 cells in the tumour area in form of induction of CD107a (FIG. 26A, p=0.0079) and Granzyme B (FIG. 26B, p=0.0159).

### Example 24—Exemplary Bispecific Antibody ‘1166/1261’ Localizes to the Tumour

### Summary

OX40 and CTLA-4 are highly expressed on T cells in the tumor environment, particularly on Tregs. Thus, OX40-CTLA-4 bispecific antibodies are expected induce tumor localization due to the dual targeting. The ability of 1166/1261 to localize to the tumor was investigated using transgenic mice for human OX40 and subcutaneous tumour models of MC38 colon carcinoma. 1166/1261 demonstrated statistically significant tumor localization compared to animals treated with vehicle or isotype control. No localization was seen in the spleen.

Material and Methods

Female mice transgenic for human OX40 (homozygous human OX40 knock-in mouse model, developed by GenOway) were used in the experiments. All experiments were done by approval of Malmö/Lund ethical committee.

MC38 colon cancer growing in log phase was injected subcutaneously (1×10⁶ cells) on day 0. The mice were given one treatment on day 17 (with 1.33 μmol Ab intraperitoneally). Twenty-four hours after the injection, the tumors and spleens were harvested, stained with a viability marker, APCeFluor780-labelled anti-CD45 and PE-labelled anti-hIgG and analysed by flow cytometry. The percentage of hIgG⁺ cells out of live CD45⁺ cells were compared between the different groups. The data was analyzed using Mann-Whitney, non-parametric, 2-tailed test.

### Results and Conclusions

The ability of 1166/1261 to induce tumor localization was investigated in hOX40tg mice using MC38 colon cancer. The experiments demonstrated that 1166/1261, but not the isotype control, was detected in the tumor after treatment (FIG. 27A). 1166/1261 could not be detected in the spleen (FIG. 27 B), suggesting localization to the tumor.

### Example 25—Combinatorial Effects of PD-1 in MC38 Colon Carcinoma Model

### Summary

Successful clinical trials with PD-1 monoclonal antibodies and other immune-checkpoint inhibitors have opened new avenues in cancer therapy. However, a large subset of cancer patients still fail to respond, prompting intensified research on combination therapies. The OX40-CTLA-4 bispecific antibody was investigated in PD-1 combination using transgenic mice for human OX40 and using MC38 colon carcinoma model.

1166/1261 alone demonstrated statistically significant anti-tumour effects in form of tumour volume inhibition and increased survival. In addition, 1166/1261 was able to enhance significantly the anti-tumour effects of PD-1 treatment. These data show the potential benefits of combining 1166/1261 with PD-1 antibodies in patients.

Material and Methods

Female homozygotes for human OX40 (hOX40tg) age 7-14w, generated by genOway, France, and bred in-house, were used in the experiments. All experiments were done by approval of Malmö/Lund ethical committee.

MC38 colon carcinoma growing in log phase was injected subcutaneously (1×10⁶ cells) on day 0 and treatments of 1166/1261 (1.33 μmol) and/or anti-mouse PD-1 (250 μg, RPM1-14, BioXcell, US) antibody were done intraperitoneally on days 7, 10 and 13. The tumour volume was measured three times a week with a caliper and the tumour volume was calculated using formula (¾×π×(width/2)×(length/2)×(height/2). The mice were terminated at ethical tumour limit. The the tumor volume was analyzed using Mann-Whitney, non-parametric, 2-tail test and survival Kaplan-Meyer, Log-Rank, using Graph Pad Prism.

Results

The combinatorial anti-tumour effects of a bispecific OX40-CTLA-4 antibody and anti PD-1 was investigated in hOX40tg mice using MC38 colon carcinoma model. The experiments demonstrated anti-tumour effects induced by 1166/1261, both in form of tumour volume inhibition and increased survival p=0.0124 (FIGS. 28A and B). The combination of 1166/1261 with PD-1 treatment significantly increased the anti-tumour effect (p<0.0001). These data demonstrated that 1166/1261 could enhance the anti-tumour effects obtained with the PD-1 antibodies in clinic.

### Example 26—Combinatorial Effects of PD-1 in CT26 Colon Carcinoma Model

### Summary

Successful clinical trials with PD-1 monoclonal antibodies and other immune-checkpoint inhibitors have opened new avenues in cancer therapy. However, a large subset of cancer patients still failures to respond, prompting intensified research on combination therapies. The OX40-CTLA bispecific antibody was investigated in PD-1 combination using transgenic mice for human OX40 and using CT26 colon carcinoma model.

1166/1261 alone demonstrated statistically significant anti-tumour effects in form of tumour volume inhibition and increased survival. In addition, 1166/1261 was able to enhance significantly the anti-tumour effects of PD-1 treatment. These data demonstrated that 1166/1261 could enhance the anti-tumour effects obtained with the PD-1 antibodies in clinic

Material and Methods

Female homozygotes for human OX40 (hOX40tg) age 7-14w, generated by genOway, France, and bred in-house, were used in the experiments. All experiments were done by approval of Malmö/Lund ethical committee.

CT26 colon carcinoma growing in log phase was injected subcutaneously (1×10⁶ cells) on day 0 and treatments of 1166/1261 (1.33 μmol) and/or anti-mouse PD-1 (250 μg, RPM1-14, BioXcell, US) antibody were done intraperitoneally on days 7, 10 and 13. The tumour volume was measured three times a week with a caliper and the tumour volume was calculated using formula (¾×π×(width/2)×(length/2)×(height/2). The the tumor volume was analyzed using Mann-Whitney, non-parametric, 2-tail test and survival Kaplan.meyer, Log-Rank, using Graph pad prism.

Results

The combinatorial anti-tumour effects of a bispecific OX40-CTLA-4 antibody and anti PD-1 was investigated in hOX40tg mice using CT26 colon carcinoma model. The experiments demonstrated statistically significant effect by 1166/1261, both as tumour volume inhibition and increased survival whereas PD-1 antibody alone did not demonstrate any potent anti-tumour efficacy (FIGS. 29A and B). Addition of 1166/1261 to PD-1 treatments significantly increased the anti-tumour efficacy of PD-1 antibody. These data demonstrated that 1166/1261 could enhance the anti-tumour effects obtained with PD-1 antibodies in clinic.

### Example 27—Anti-Tumour Efficacy of 1166/1261 Against PANC02 Pancreas Cancer

### Summary

Pancreatic adenocarcinoma is an aggressive type of cancer. Patients with pancreatic cancer typically have very poor prognosis and the cancer type is the fourth most common cause of death from cancer in the United States. The anti-tumour effects of 1166/1261 were examined against PANC02 pancreatic cancer using transgenic mice for hOX40.

1166/1261 demonstrated statistically significant anti-tumour efficacy in form of tumour volume inhibition and increased survival.

Material and Methods

Female mice knock-in for human OX40 (hOX40tg), generated by genOway, were used in the experiments. All experiments were approved by the Malmo/Lund Ethical Committee.

PANC02 pancreatic cancer growing in log phase was injected subcutaneously (0.25×10⁶ cells) in on day 0 and treatments with 1166/1261 (1.33 μmol) were done intraperitoneally on days 7, 10 and 13. The tumor volume was analyzed using Mann-Whitney, non-parametric, 2-tail test and survival Kaplan.meyer, Log-Rank, using Graph pad prism.

Results

The anti-tumour efficacy of 1166/1261 against pancreatic cancer was examined using hOX40 transgenic mice and PANC02 pancreatic cancer. 1166/1261 demonstrated significant anti-tumour efficacy in form of tumour growth inhibition (FIG. 30A) and increased survival (FIG. 30B).

### Example 28—1166/1261 Restores T Cell Activation Through CTLA-4 Blockade

### Summary

The ability of 1166/1261 to block CTLA-4 and increase the co-stimulation and activation of T cells was investigated in a CTLA-4 blockade reporter assay. 1166/1261 was able to block CD80/CD86 binding to CTLA-4 and restore co-stimulation via CD28 leading to T cell activation.

Material and Methods

The assay uses Raji cells expressing a TCR activator and CD80/CD86 to co-stimulate Jurkat reporter T cells expressing TCR, CTLA-4 and CD28 connected with an IL2 promotor with a luc2P element. Activation is measured as luminescence. In the absence of CTLA-4 antibodies, CD80/CD86 binds to CTLA-4 with higher affinity than to CD28, thus blocking the signal. By adding CTLA-4-binding antibodies, CD80/CD86 binding to CTLA-4 will be blocked, and as a consequence, co-stimulation of T cells via CD28 will increase leading to T cell activation.

Serially diluted 1166/1261 and isotype control were immobilized to the plate and incubated overnight followed by addition of Jurkat reporter cells and Raji cells. After overnight incubation, T cell activation was measured as luminescence.

### Results and Conclusions

As shown in FIG. 31, 1166/1261 is able to restore T cell activation, whereas no effect is seen with the isotype control.

### Example 29—T Cell Activation Induced by 1166/1261 Upon CTLA-4 Crosslinking

### Summary

The ability of 1166/1261 to activate T cells was investigated in serial agonistic assays upon CTLA-4 crosslinking. 1166/1261 demonstrated agonistic T cell activation in form of secretion of IFN-γ, IL-2, or increased T cell proliferation.

Material and Methods

Human CD3⁺ or CD4⁺ T cells were purified from PBMCs obtained from leukocyte filters from the blood bank of the Lund University Hospital using negative selection (Pan T cell Isolation Kit or CD4+ T cell Isolation Kit, Miltenyi), and cultured together with serially diluted 1166/1261, a mix of monospecific antibodies 1166/1167+ IsoCtr/1261 or isotype control.

IFN-γ release by CD3⁺ T-cells was measured after culturing T cells (100,000 cells/well) with test antibodies in plates pre-coated with CTLA-4 (Orencia, 5 μg/ml) and αCD3 (OKT3, 3 μg/ml). After a 72-h incubation period, the level of IFN-γ was measured in the supernatants by ELISA.

Release of IL-2 by CD4⁺ T cells was measured by culturing T cells (50,000 cells/well) with test antibodies in plates containing irradiated HEK cells (30,000 cells/well) stably expressing CTLA-4 (800,000 receptors/cell) and αCD3 beads (UCHT-1, cell:bead ratio 1:1.1). After 72 h, the level of IL-2 was measured in the supernatants by ELISA.

Proliferation was determined by culturing CD4⁺ T cells (50,000 cells/well) with test compound in plates pre-coated with CTLA-4 (Orencia, 5 μg/ml) and αCD3 (UCHT-1, 0.1 μg/ml). After 72 h the proliferation was measured with CellTitre Glow (Promega).

### Results and Conclusions

The agonistic activity of 1166/1261 was investigated in differential T cell activation. The results in FIG. 32 demonstrate dose-dependent effects of 1166/1261 in terms of IFN-γ and IL-2 release as well as proliferation, while this is not seen with the combination of monospecific OX40 and CTLA-4 antibodies. The results indicate that 1166/1261 has a strong agonistic effect upon CTLA-4 crosslinking.

### Example 30—T Cell Activation Induced by 1166/1261 Upon FcγR Crosslinking

### Summary

The ability of 1166/1261 to activate T cells was investigated in agonistic assays upon FcγR crosslinking. 1166/1261 demonstrated agonistic T cell activation in the form of IL-2 secretion.

Material and Methods

CHO cells stably transfected to express CD64 (FcγRI) were irradiated, plated (100,000 cells/well) and allowed to adhere overnight. Serially diluted 1166/1261, the combination of monospecific antibodies (1166/1167+Ctr IgG/1261) or isotype control were added to the wells. Beads coated with αCD3 (OKT3) were added for suboptimal T cell activation. Human CD4⁺ T cells were purified from PBMCs obtained from leukocyte filters from the blood bank of the Lund University Hospital using negative selection (CD4+ T cell Isolation Kit, Miltenyi) and added to the wells (50,000 cells/well). After a 72-h incubation period, IL-2 secretion was measured by ELISA. In total, 8 donors were tested in the assay.

### Results and Conclusions

As shown in FIG. 33, a dose-dependent activation of 1166/1261 is demonstrated, while this is not seen with the combination of monospecific OX40 and CTLA-4 antibodies. The results indicate that 1166/1261 has a strong agonistic effect upon FcγR crosslinking.

### Example 31—Ability of 1166/1261 to Deplete Primary Tregs

### Summary

In the tumour environment, regulatory T cells have a high expression of both OX40 and CTLA-4. OX40-CTLA-4 bispecific antibodies are expected to induce ADCC of target-expressing cells, especially in the tumour environment. The ability of 1166/1261 to induce ADCC was examined using an ADCC Reporter assay specific for human Fc₇R111 (V158) as a surrogate for ADCC. 1166/1261 demonstrated significant activation of effector cells and this induction was superior in activity compared to the monoclonal counterparts alone (data not shown) or in combination.

Material and Methods

An FcγRIIIa (V158) ADCC Reporter assay (Promega) was used to determinate ADCC induction. CD4⁺CD25⁺CD127^{low} Tregs were isolated by negative selection using the EasySep™ Human CD4⁺CD127^{low} CD25⁺ Regulatory T Cell Isolation Kit (Stemcell Technologies). Tregs were activated for 48 h in the presence of αCD3/αCD28 Dynabeads (Gibco) to upregulate target expression and used as target cells. Serially diluted 1166/1261, the combination of monospecific antibodies (1166/1167+Ctr IgG/1261) or isotype control were cultured together with effector and target cells (5:1 ratio) for 6 hours. The expression of OX40 and CTLA-4 was determined before and after culture by flow cytometry.

### Results and Conclusions

The ability of 1166/1261 to induce depletion of regulatory T cells in vitro was assessed by ADCC reporter assay. As shown in FIG. 34A, 1166/1261 has the ability to induce ADCC in primary human Tregs. The induction was markedly higher than with the monoclonal counterparts alone (data not shown) or with a mixture of them. The results correlated with the expression levels of OX40 and CTLA-4. Fresh or unstimulated Tregs expressed low levels of OX40 and CTLA-4, whereas the levels were clearly up-regulated after activation with αCD3/αCD28 (FIG. 34B).

### Example 32—Ability of 1166/1261 to Deplete Primary Tregs

### Summary

In the tumour environment, regulatory T cells have a high expression of both OX40 and CTLA-4. OX40-CTLA-4 bispecific antibodies are expected to induce ADCC of target-expressing cells, especially in the tumour environment. The ability of 1166/1261 to induce ADCC was examined using an LDH release assay with allogeneic NK cells as effector cells. 1166/1261 induced a strong NK cell-mediated lysis of the Tregs.

Material and Methods

CD4⁺CD25⁺CD127^{low} Tregs were isolated by negative selection using the EasySep™ Human CD4⁺CD127^{low} CD25⁺ Regulatory T Cell Isolation Kit (Stemcell Technologies). Tregs were activated for 48 h in the presence of αCD3/αCD28 Dynabeads (Gibco) to upregulate target expression and thereafter used as target cells. As effector cells, allogeneic NK cells isolated from PBMC using negative selection using the EasySep™ Human NK Cell Isolation Kit (Stemcell Technologies) were used. Effector cells and target cells were cultured at a ratio of 15:1 together with serially diluted 1166/1261 or isotype control for 4 h. Thereafter, the level of LDH in the supernatant was measured.

### Results and Conclusions

As shown in FIG. 35, 1166/1261 has the ability to induce ADCC in primary human Tregs in a dose-dependent manner. No effect was seen with the isotype control.

### Example 33—Assessment of 1166/1621 in Pilot Cynomolgus Tox Study

### Summary

A single dose, dose-range finding study was carried out in cynomolgus monkeys. The number of early (CD25+) and late (CD69+) T cells and proliferating central memory (CD197+CD45RA-Ki67+) were found to increase after a single dose of 1166/1261.

Materials and Methods

A single dose, dose-range finding study was carried out in cynomolgus monkeys. The dose groups consisted of one male and one female. All animals were over 2.5 years of age. Dose groups 1-3 received 3; 10; or 30 mg/kg of 1166/1261 respectively, as intravenous infusions over 1 hour. Dose group 4 received a subcutaneous injection of 30 mg/kg.

Whole blood samples were taken at pre-dose (two occasions), and post dose at Day 2, 8, 15, 29 and 43 for peripheral blood immunephenotyping. Truecount tubes (BD Biosciences) were used for enumeration and antibodies for stainings were purchased from BD Biosciences or Biolegend. Differences in cell populations were compared by calculating the fold-difference of post dose samples to the average of the two pre-dose samples for each individual.

### Results and Conclusions

Cynomolgus monkeys receiving a single dose of 1166/1261 demonstrated effects on the number of early (CD69+) and late (CD25+) T cells (FIG. 36B) and proliferating central memory (CD197+CD45RA-Ki67+) T cells (FIG. 36A) were found to increase. No distinguishable difference was seen between the four dose groups.

1. A bispecific polypeptide comprising a first binding domain, designated B1, which is capable of specifically binding to OX40, and a second binding domain, designated B2, which is capable of specifically binding to CTLA-4.

2. A polypeptide according to claim 1, wherein the first and/or second binding domains are selected from the group consisting of: antibodies or antigen-binding fragments thereof

3. A polypeptide according to claim 2 wherein the antigen-binding fragment is selected from the group consisting of: an Fv fragment (such as a single chain Fv fragment, or a disulphide-bonded Fv fragment), a Fab-like fragment (such as a Fab fragment; a Fab′ fragment or a F(ab)₂ fragment) and domain antibodies.

4. A polypeptide according to any one of the preceding claims wherein the polypeptide is a bispecific antibody.

5. A polypeptide according to claim 4 wherein:
(a) binding domain B1 and/or binding domain B2 is an intact IgG antibody;
(b) binding domain B1 and/or binding domain B2 is an Fv fragment;
(c) binding domain B1 and/or binding domain B2 is a Fab fragment; and/or
(d) binding domain B1 and/or binding domain B2 is a single domain antibody.

6. A polypeptide according to any one of the preceding claims wherein the bispecific polypeptide is selected from the groups consisting of:
(a) bivalent bispecific antibodies, such as IgG-scFv bispecific antibodies (for example, wherein B1 is an intact IgG and B2 is an scFv attached to B1 at the N-terminus of a light chain and/or at the C-terminus of a light chain and/or at the N-terminus of a heavy chain and/or at the C-terminus of a heavy chain of the IgG, or vice versa);
(b) monovalent bispecific antibodies, such as a DuoBody® or a ‘knob-in-hole’ bispecific antibody (for example, an scFv-KIH, scFv-KIH^{r}, a BiTE-KIH or a BiTE-KIH^{r};
(c) scFv₂-Fc bispecific antibodies (for example, ADAPTIR™ bispecific antibodies);
(d) BiTE/scFv₂ bispecific antibodies;
(e) DVD-Ig bispecific antibodies or other IgG-FAb, FAb-IgG bispecific antibodies regardless of bivalency or linkers/connectors employed;
(f) DART-based bispecific antibodies (for example, DART-Fc, DART₂-Fc or DART);
(g) DNL-Fab₃ bispecific antibodies; and
(h) scFv-HSA-scFv bispecific antibodies.

7. A polypeptide according to any one of the preceding claims comprising a human Fc region or a variant of a said region, where the region is an IgG1, IgG2, IgG3 or IgG4 region, preferably an IgG1 or IgG4 region.

8. A polypeptide according to any of the preceding claims, wherein the polypeptide is capable of inducing antibody dependent cell cytotoxicity (ADCC), antibody-dependent cellular phagocytosis (ADCP), complement-dependent cytotoxicity (CDC), and/or apoptosis.

9. A polypeptide according to any one of the preceding claims, wherein:
B1 is an antibody, or antigen binding fragment thereof, specific for OX40; and
B2 is a polypeptide binding domain specific for CTLA-4, which comprises or consists of:
i) the amino acid sequence of SEQ ID NO: 3; or
ii) an amino acid sequence in which at least one amino acid is changed when compared to the amino acid sequence of SEQ ID NO: 3 provided that said binding domain binds to human CTLA-4 with higher affinity than wild-type human CD86.

10. A polypeptide according to any one of the preceding claims in which the CTLA-4 specifically bound by the polypeptide is primate or murine, preferably human, CTLA-4, and/or wherein the OX40 specifically bound by the polypeptide is primate, preferably human, OX40.

11. A polypeptide according to any one of the preceding claims in which B1 comprises at least one heavy chain (H) and/or at least one light chain (L) and B2 is attached to said at least one heavy chain (H) or least one light chain (L).

12. A polypeptide according to claim 11 in which B1 comprises:
at least one heavy chain (H) and at least one light chain (L) and B2 is attached to either the heavy chain or the light chain; or
two identical heavy chains (H) and two identical light chains (L) and B2 is attached to both heavy chains or to both light chains.

13. A polypeptide according to any one of the preceding claims which comprises or consists of a polypeptide chain arranged according to any one of the following formulae, written in the direction N-C:
L-(X)n-B2; (A)
B2-(X)n-L; (B)
B2-(X)n-H; (C)
H—(X)n-B2; (D)
wherein X is a linker and n is 0 or 1.

14. A polypeptide according to claim 13, wherein X is a peptide with the amino acid sequence SGGGGSGGGGS (SEQ ID NO: 47), SGGGGSGGGGSAP (SEQ ID NO: 48), NFSQP (SEQ ID NO:49), KRTVA (SEQ ID NO: 50), GGGGSGGGGSGGGGS (SEQ ID NO: 144) or (SG)m, where m=1 to 7.

15. A polypeptide according to any one of the preceding claims, which binds to human OX40 with a Kd of less than 50×10⁻¹⁰M, 25×10⁻¹⁰M, or 20×10⁻¹⁰M and/or which binds to human CTLA-4 with a Kd value which is less than 60×10⁻⁹M, 25×10⁻⁹M, or 10×10⁻⁹M.

16. A polypeptide according to any one of the preceding claims, which induces an increase in the activity of an effector T cell, preferably a CD4+ effector T cell, optionally wherein said increase is at least 1.5 fold, 4.5 fold or 7 fold higher than the increase in activity of an effector T cell induced by a combination of B1 and B2 administered to the T cell as separate molecules.

17. A polypeptide according to claim 16, wherein said increase in T cell activity is an increase in proliferation and/or IL-2 production by the T cell.

18. A polypeptide according to any one of the preceding claims which competes for binding to OX40 with antibody 1166/1167; and/or which competes for binding to CTLA-4 with CD86 mutant 1040 (SEQ ID NO: 17).

19. A polypeptide according to any one of the preceding claims which competes for binding to OX40 with antibody 1166/1261, and/or which competes for binding to CTLA-4 with antibody 1166/1261.

20. A polypeptide according to any one of the preceding claims, wherein 1, 2, 3, 4, 5, 6, 7, 8, 9 or 10 amino acids in said amino acid sequence of B2 (ii) are substituted when compared to the amino acid sequence of SEQ ID NO: 3; optionally wherein there are no insertions or deletions compared to the amino acid sequence of SEQ ID NO: 3.

21. A polypeptide according to claim 20, wherein at least one of said amino acid substitutions in said amino acid sequence of said first binding domain is at position 122, and optionally wherein said amino acid sequence is also substituted in at least one of positions 107, 121 and 125.

22. A polypeptide according to any one of the preceding claims wherein said amino acid sequence of B2 comprises or consists of an amino acid sequence selected from any one of SEQ ID NOs 6 to 24 (as shown in Table C), or variants of said sequences which have more than 60%, or more than 70%, e.g. 75 or 80%, preferably more than 85%, e.g. more than 90 or 95% amino acid identity to the amino acid sequences of SEQ ID NOs 6 to 24.

23. A polypeptide according to any one of the preceding claims wherein said amino acid sequence of B2 comprises or consists of the amino acid sequence of SEQ ID NO: 17 (CD86 mutant 1040, as shown in Table C).

24. A polypeptide according to any one of the preceding claims, wherein B1 exhibits at least one of the following functional characteristics when present independently of B2:
I. binding to human OX40 with a K_{D} value which is less than 10×10⁻¹⁰M, more preferably less than 5×10⁻¹⁰M;
II. does not bind to murine OX40; and
III. does not bind to other human TNFR superfamily members, for example human CD137 or CD40

25. A polypeptide according to any one of the preceding claims, wherein B1 comprises any one, two, three, four, five or all six features independently selected from the following:
(a) a heavy chain CDR1 sequence which is 8 amino acids in length and comprises the consensus sequence: “G, F, T, F, G/Y/S, G/Y/S, Y/S, Y/S/A”;
(b) a heavy chain CDR2 sequence which is 8 amino acids in length and comprises the consensus sequence: “I, G/Y/S/T, G/S/Y, S/Y, G/S/Y, G/S/Y, G/S/Y, T”;
(c) a heavy chain CDR3 sequence which is 9 to 17 amino acids in length and which comprises the consensus sequence of: “A, R, G/Y/S/H, G/Y/F/V/D, G/Y/P/F, −/H/S, −/N/D/H, −/Y/G, −/Y, −/Y, −/W/A/V, −/A/Y, −/D/A/Y/G/H/N, Y/S/W/A/T, L/M/I/F, D, Y”
(d) a light chain CDR1 sequence which consists of the sequence: “Q, S, I, S, S, Y”;
(e) a light chain CDR2 sequence which consists of the sequence: “A, A, S”;
(f) a light chain CDR3 sequence which is 8 to 10 amino acids in length and comprises the consensus sequence: “Q,Q, S/Y/G, −/Y/H/G, −/S/Y/G/D/W, S/Y/G/D, S/Y/G/T, P/L, Y/S/H/L/F, T”;
wherein the heavy chain CDR3 sequence of (c) is preferably a sequence of 10 amino acids in length which comprises the consensus sequence “A, R, Y/H, D, Y, A/Y/G, S/W/A, M/L, D, Y”; and
the light chain CDR3 sequence of (f) preferably consists of the sequence “Q, Q, Y, Y, W, Y, G, L, S, T”.

26. A polypeptide according to any one of the preceding claims, wherein B1 comprises all three heavy chain CDR sequences of a VH sequence as shown in Table A(1) and/or all three light chain CDR sequences of a VL sequence as shown in Table A(2) or wherein B1 comprises a heavy chain VH sequence and/or a light chain VL sequence as shown in Table B.

27. A polypeptide according to any one of the preceding claims, wherein B1 comprises a heavy chain CDR3 sequence of 11 amino acids in length which comprises the consensus sequence “A, R, Y/H, D, Y, A/Y/G, S/W/A, M/L, D, Y” and the light chain VL sequence of SEQ ID NO: 89 (1167 as shown in Table B), optionally wherein the light chain VL sequence of SEQ ID NO: 89 is present as part of the longer sequence of SEQ ID NO: 125 (1261 as shown in Table D).

28. A polypeptide according to any one of the preceding claims, wherein binding domain B1 comprises the light chain VL sequence of SEQ ID NO: 89 (1167 as shown in Table B), and the heavy chain VH sequence of SEQ ID NO: 91 (1166 as shown in Table B), or variants of said sequences which have more than 60%, or more than 70%, e.g. 75 or 80%, preferably more than 85%, e.g. more than 90 or 95% amino acid identity to SEQ ID NO: 89 and/or SEQ ID NO: 91.

29. A polypeptide according to any one of the preceding claims, wherein B1 comprises an human Fc region or a variant of a said region, where the region is an IgG1, IgG2, IgG3 or IgG4 region, preferably an IgG1 or IgG4 region.

30. A polypeptide according to any one of the preceding claims, which comprises or consists of the amino acid sequence of any one of SEQ ID NOs 125 to 134, optionally wherein said polypeptide is a provided as a component part of an antibody, or variants of said sequences which have more than 60%, or more than 70%, e.g. 75 or 80%, preferably more than 85%, e.g. more than 90 or 95% amino acid identity to the amino acid sequences of SEQ ID NOs 125 to 134.

31. A polypeptide according to any one of the preceding claims, which comprises or consists of the amino acid sequence of SEQ ID NO 125 (1261 as shown in Table D).

32. A polypeptide according to any one of the preceding claims, which comprises or consists of the amino acid sequence of SEQ ID NO 125 and/or the amino acid sequence of SEQ ID NO: 91, or variants of said sequences which have more than 60%, or more than 70%, e.g. 75 or 80%, preferably more than 85%, e.g. more than 90 or 95% amino acid identity to SEQ ID NO: 125 and/or SEQ ID NO: 91.

33. A polypeptide according to any one of the preceding claims, which comprises or consists of an amino acid sequence of SEQ ID NO: 125 (1261 as shown in Table D). and an amino acid sequence of SEQ ID NO: 91 (1161, as shown in Table B).

34. A polypeptide according to any one of the preceding claims, wherein the bispecific polypeptide is capable of inducing a synergistic increase in the intratumoural CD8/Treg ration compared to the combined effect of the individual monospecific counterpart polypeptides.

35. A bispecific polypeptide according to any one of the preceding claims for use as a medicament.

36. Use of a bispecific polypeptide according to any one of claims 1 to 34 in the manufacture of a medicament.

37. A method of treating or preventing a disease or condition in an individual, the method comprising administering to an individual a bispecific polypeptide according to any one of the preceding claims.

38. A bispecific polypeptide according to claim 35 or a use according to claim 36 or a method according to claim 37 wherein the disease or condition is cancer and optionally wherein the individual is human.

39. A bispecific polypeptide or use or method according to claim 38, wherein the method comprises administering the bispecific polypeptide systemically or locally, such as at the site of a tumour or into a tumour draining lymph node, or wherein the bispecific polypeptide is for administration systemically or locally, such as at the site of a tumour or into a tumour draining lymph node.

40. A bispecific polypeptide or method or use according to claim 38 or 39 wherein the cancer is prostate cancer, breast cancer, colorectal cancer, pancreatic cancer, ovarian cancer, lung cancer, cervical cancer, rhabdomyosarcoma, neuroblastoma, multiple myeloma, leukemia, acute lymphoblastic leukemia, melanoma, bladder cancer, gastric cancer, head and neck cancer, liver cancer, skin cancer, lymphoma or glioblastoma.

41. A bispecific polypeptide or method or use for according to any one of claims 38 to 40 wherein the polypeptide is for use in combination with one or more additional therapeutic agents.

42. A bispecific polypeptide or method or use for according to claim 41 wherein the one or more additional therapeutic agents is/are an immunotherapeutic agent that binds a target selected from the group consisting of PD-1/PD-L1, CD137, CD40, GITR, LAG3, TIM3, CD27 and KIR.

43. A bispecific polypeptide or method or use for according to claim 42 wherein the one or more additional therapeutic agents is/are an immunotherapeutic agent that binds PD-1 or PD-L1, such as an anti-PD-1 antibody or an anti-PD-L1 antibody.

44. A polynucleotide encoding at least one polypeptide chain of a bispecific polypeptide according to any one of claims 1 to 34.

45. A composition comprising a bispecific polypeptide according to any one of claims 1 to 34 and at least one pharmaceutically acceptable diluent or carrier.

46. A polypeptide according to any one of claims 1 to 34 conjugated to an additional therapeutic moiety.


SEQUENCE LISTING (161 sequences)

SEQ ID NO: 1 - protein, 137 aa, Homo sapiens
  Human CTLA-4
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLCENAPNRARM

SEQ ID NO: 2 - protein, 220 aa, Homo sapiens
  Human CD28
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - protein, 111 aa, Homo sapiens
  Monomeric extracellular domain of human wildtype CD86
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLA

SEQ ID NO: 4 - protein, 247 aa, Homo sapiens
  Monomeric extracellular and transmembrane domains of human wildtype CD86
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 5 - protein, 111 aa, Artificial Sequence
  Mutant form of the extracellular domain of human CD86
APLKIQAYFNETADLPCQFANSQNQSLSELVVFWQDQENLVLNEVYLGKEKFDSVASKYMGRTSFDSDSWTLRLHNLQIKDKGLYQCIIHHKKPTGMIRIHQMNSELSVLA

SEQ ID NO: 6 - protein, 109 aa, Artificial Sequence
  CD86 variant 900
LKIQAYFNETADLPCQFANSQNQSLSELVVFWQDQENLVLNEVYLGKEKFDSVDSKYMGRTSFDSDSWTLRLHNLQIKDKGIYQCVIHHKKPSGLVKIHEMNSELSVLA

SEQ ID NO: 7 - protein, 109 aa, Artificial Sequence
  CD86 variant 901
LKIQAYFNETADLPCQFANSQNLTLSELVVFWQDQENLVLNEVYLGKEKFDSVHSKYMGRTSFDSDSWTLRLHNLQIKDKGIYQCVIHHKKPTGMIKIHEMNSELSVLT

SEQ ID NO: 8 - protein, 109 aa, Artificial Sequence
  CD86 variant 904
LKIQAYFNETADLPCQFANSQNQSLSELIVFWQDQENLVLNEVYLGKERFDAVDSKYMGRTSFDSDSWTLRLHNLQIKDKGIYQCIIHHKKPSGMVKIHQMDSELSVLA

SEQ ID NO: 9 - protein, 109 aa, Artificial Sequence
  CD86 variant 906
LKIQAYINETADLPCQFANSQNLSLSELVVFWQDQENLVLNEVYLGKERFDSVDSKYMGRTSFDSDSWTLRLHNLQIKDKGFYQCIIHHKKPTGLVKIHEMNSELSVLA

SEQ ID NO: 10 - protein, 109 aa, Artificial Sequence
  CD86 variant 907
LKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIKIHEMNSELSVLA

SEQ ID NO: 11 - protein, 109 aa, Artificial Sequence
  CD86 variant 908
LKIQAYFNETADLPCQFANSQNQSLSELVVFWQDQENLVLNEVYLGKEKFDSVHSKYMGRTSFDSDSWTLRLHNLQIKDKGIYQCIIHHKKPTGMVKIHEMNSELSVLA

SEQ ID NO: 12 - protein, 109 aa, Artificial Sequence
  CD86 variant 910
LKIQAYFNETADLPCQFANSQNQSLSELVVFWQDQENLVLNEVYLGKEKFDSVDSKYMGRTSFDSDSWTLRLHNLQIKDKGIYQCIIHHKKPTGMVKIHEMNSELSVLA

SEQ ID NO: 13 - protein, 109 aa, Artificial Sequence
  CD86 variant 915
LKIQAYFNETADLPCQFANSQNQSLSELVVFWQDQENLILNEVYLGKEKFDSVDSKYMGRTSFDSDSWTLRLHNLQIKDKGFYQCIIHHKKPSGLIKIHQMDSELSVLA

SEQ ID NO: 14 - protein, 109 aa, Artificial Sequence
  CD86 variant 938
LKIQAYFNETADLPCQFANSQNQSLSELVVFWQDQENLILNEVYLGKEKFDSVHSKYMGRTSFDSDSWTLRLHNLQIKDKGIYQCIIHHKKPTGMVKIHQMNSELSVLA

SEQ ID NO: 15 - protein, 111 aa, Artificial Sequence
  CD86 variant 1038
APLKIQAYFNETADLPCQFANSQNLSLSELVVFWQDQENLVLNEVYLGKEKFDSVDSKYMGRTSFDSDSWTLRLHNLQIKDKGIYQCIIHHKKPTGMVKIHEMNSELSVLA

SEQ ID NO: 16 - protein, 111 aa, Artificial Sequence
  CD86 variant 1039
APLKIQAYFNETADLPCQFANSQNLSLSELVVFWQDQENLVLNEVYLGKEKFDSVSSKYMGRTSFDSDSWTLRLHNLQIKDKGIYQCIIHHKKPSGMVKIHQMDSELSVLA

SEQ ID NO: 17 - protein, 111 aa, Artificial Sequence
  CD86 variant 1040
APLKIQAYFNETADLPCQFANSQNLSLSELVVFWQDQENLVLNEVYLGKERFDSVDSKYMGRTSFDSDSWTLRLHNLQIKDKGRYQCIIHHKKPTGMINIHQMNSELSVLA

SEQ ID NO: 18 - protein, 111 aa, Artificial Sequence
  CD86 variant 1041
APLKIQAYLNETADLPCQFANSQNLSLSELVVFWQDQENLVLNEVYLGKEKFDSVDSKYMGRTSFDSDSWTLRLHNLQIKDKGIYQCIIHHKKPTGLVKIHEMNSELSVLA

SEQ ID NO: 19 - protein, 111 aa, Artificial Sequence
  CD86 variant 1042
APLKIQAYFNETADLPCQFANSQNLSLSELVVFWQDQENLVLNEVYLGKEIFDSVSSKYMGRTSFDSDSWTLRLHNLQIKDKGIYQCIIHHKKPSGMVKIHQMDSELSVLA

SEQ ID NO: 20 - protein, 111 aa, Artificial Sequence
  CD86 variant 1043
APLKIQAYFNETADLPCQFANSQNLSLSELVVFWQDQENLVLNEVYLGKEKFDSVDSKYMGRTSFDSDSWTLRLHNLQIKDKGIYQCIIHHKKPTGMIKIHEMNSELSVLA

SEQ ID NO: 21 - protein, 111 aa, Artificial Sequence
  CD86 variant 1044
APLKIQAYFNETADLPCQFANSQNLTLSELVVFWQDQENLVLNEVYLGKEKFDSVSSKYMGRTSFDSDSWTLRLHNLQIKDKGIYQCIIHHKKPTGMIKIHEMSSELSVLA

SEQ ID NO: 22 - protein, 111 aa, Artificial Sequence
  CD86 variant 1045
APLKIQAYFNETADLPCQFANSQNLTLSELVVFWQDQENLVLNEVYLGKEKFDSVDSKYMGRTSFDSDSWTLRLHNLQIKDKGLYQCIIHHKKPTGLVKIHEMNSELSVLA

SEQ ID NO: 23 - protein, 111 aa, Artificial Sequence
  CD86 variant 1046
APLKIQAYFNETADLPCQFANSQNQSLSELVVFWQDQENLVLNEVYLGKEKFDSVDSKYMGRTSFDSDSWTLRLHNLQIEDKGIYQCIIHHKKPSGMVKIHQMDSELSVLA

SEQ ID NO: 24 - protein, 111 aa, Artificial Sequence
  CD86 variant 1047
APLKIQAYFNETADLPCQFANSQNLSLSELVVFWQDQENLVLNEVYLGKEKFDSVDSKYMGRTSFDSDSWTLRLHNLQIKDKGIYQCIIHHKKPTGLVKIHEMNSELSVLA

SEQ ID NO: 25 - DNA, 327 bp, Artificial Sequence
  CD86 variant 900
ctcaaaatccaagcgtacttcaacgaaactgcagacttaccgtgtcagtttgccaattcgcagaatcaaagcctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcaaagataagggtatctaccagtgcgtgatccaccataagaagccgagcggtctggtgaagattcacgagatgaactccgagttgtctgtcctggcg

SEQ ID NO: 26 - DNA, 327 bp, Artificial Sequence
  CD86 variant 901
ctcaaaatccaagcgtacttcaacgaaactgcagacttaccgtgtcagtttgccaattcgcagaatctgaccctgagcgaactggtggttttctggcaggatcaggagaacctggttctgaacgaagtctatctgggcaaagagaaattcgacagcgtgcatagcaagtatatgggccgcaccagctttgatagcgacagctggaccctgcgtctgcacaatctgcaaatcaaagataagggtatctaccagtgcgtgatccaccataagaagccgacgggtatgattaagattcacgagatgaactccgagttgtctgtcctgacc

SEQ ID NO: 27 - DNA, 327 bp, Artificial Sequence
  CD86 variant 904
ctcaaaatccaagcgtacttcaacgaaactgcagacttaccgtgtcagtttgccaattcgcagaatcaaagcctgagcgaactgatcgttttctggcaggatcaggagaacctggttctgaacgaagtctatctgggcaaagagcggttcgacgccgtggacagcaagtatatgggccgcaccagctttgatagcgacagctggaccctgcgtctgcacaatctgcaaatcaaagataagggtatctaccagtgcattatccaccataagaagccgagcggtatggtgaagattcaccaaatggactccgagttgtctgtcctggcg

SEQ ID NO: 28 - DNA, 327 bp, Artificial Sequence
  CD86 variant 906
ctcaaaatccaagcgtacatcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtttctaccagtgcattatccaccataagaagccgacgggtctggtgaagattcacgagatgaactccgagttgtctgtcctggcg

SEQ ID NO: 29 - DNA, 327 bp, Artificial Sequence
  CD86 variant 907
ctcaaaatccaagcgtacttcaacgaaactgcagacttaccgtgtcagtttgccaattcgcagaatcaaagcctgagcgaactggtggttttctggcaggatcaggagaacctggttctgaacgaagtctatctgggcaaagagaaattcgacagcgtgcatagcaagtatatgggccgcaccagctttgatagcgacagctggaccctgcgtctgcacaatctgcaaatcaaagataagggtctgtaccagtgcattatccaccataagaagccgacgggtatgattaagattcacgagatgaactccgagttgtctgtcctggcg

SEQ ID NO: 30 - DNA, 327 bp, Artificial Sequence
  CD86 variant 908
ctcaaaatccaagcgtacttcaacgaaactgcagacttaccgtgtcagtttgccaattcgcagaatcaaagcctgagcgaactggtggttttctggcaggatcaggagaacctggttctgaacgaagtctatctgggcaaagagaaattcgacagcgtgcatagcaagtatatgggccgcaccagctttgatagcgacagctggaccctgcgtctgcacaatctgcaaatcaaagataagggtatctaccagtgcattatccaccataagaagccgacgggtatggtgaagattcacgagatgaactccgagttgtctgtcctggcg

SEQ ID NO: 31 - DNA, 327 bp, Artificial Sequence
  CD86 variant 910
ctcaaaatccaagcgtacttcaacgaaactgcagacttaccgtgtcagtttgccaattcgcagaatcaaagcctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcaaagataagggtatctaccagtgcattatccaccataagaagccgacgggtatggtgaagattcacgagatgaactccgagttgtctgtcctggcg

SEQ ID NO: 32 - DNA, 327 bp, Artificial Sequence
  CD86 variant 915
ctcaaaatccaagcgtacttcaacgaaactgcagacttaccgtgtcagtttgccaattcgcagaatcaaagcctgagcgaactggtggttttctggcaggatcaggagaacctgatcctgaacgaagtctatctgggcaaagagaaattcgacagcgtggacagcaagtatatgggccgcaccagctttgatagcgacagctggaccctgcgtctgcacaatctgcaaatcaaagataagggtttctaccagtgcattatccaccataagaagccgagcggtctgattaagattcaccaaatggactccgagttgtctgtcctggcg

SEQ ID NO: 33 - DNA, 327 bp, Artificial Sequence
  CD86 variant 938
ctcaaaatccaagcgtacttcaacgaaactgcagacttaccgtgtcagtttgccaattcgcagaatctgagcctgagcgaactggtggttttctggcaggatcaggagaacctgatcctgaacgaagtctatctgggcaaagagcggttcgacagcgtgcatagcaagtatatgggccgcaccagctttgatagcgacagctggaccctgcgtctgcacaatctgcaaatcaaagataagggtctgtaccagtgcattatccaccataagaagccgagcggtatggtgaagattcacgagatgaactccgagttgtctgtcctggcg

SEQ ID NO: 34 - DNA, 333 bp, Artificial Sequence
  CD86 variant 1038
gcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcaaagataagggtatctaccagtgcattatccaccataagaagccgacgggtatggtgaagattcacgagatgaactccgagttgtctgtcctggcg

SEQ ID NO: 35 - DNA, 333 bp, Artificial Sequence
  CD86 variant 1039
gcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagaaattcgacagcgtgagtagcaagtatatgggccgcaccagctttgatagcgacagctggaccctgcgtctgcacaatctgcaaatcaaagataagggtatctaccagtgcattatccaccataagaagccgagcggtatggtgaagattcaccaaatggactccgagttgtctgtcctggcg

SEQ ID NO: 36 - DNA, 333 bp, Artificial Sequence
  CD86 variant 1040
gcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtaggtaccagtgcattatccaccataagaagccgacgggtatgattaatattcaccaaatgaactccgagttgtctgtcctggcg

SEQ ID NO: 37 - DNA, 333 bp, Artificial Sequence
  CD86 variant 1041
gcccccctcaaaatccaagcgtacctcaacgaaactgcagacttaccgtgtcagtttgccaattcgcagaatctgagcctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcaaagataagggtatctaccagtgcattatccaccataagaagccgacgggtctggtgaagattcacgagatgaactccgagttgtctgtcctggcg

SEQ ID NO: 38 - DNA, 333 bp, Artificial Sequence
  CD86 variant 1042
gcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagattttcgacagcgtgagtagcaagtatatgggccgcaccagctttgatagtgacagctggaccctgcgtctgcacaatctgcaaatcaaagataagggtatctaccagtgcattatccaccataagaagccgagcggtatggtgaagattcaccaaatggactccgagttgtctgtcctggcg

SEQ ID NO: 39 - DNA, 333 bp, Artificial Sequence
  CD86 variant 1043
gcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagaaattcgacagcgtggatagcaagtatatgggccgcaccagctttgatagcgacagctggaccctgcgtctgcacaatctgcaaatcaaagataagggtatctaccagtgcattatccaccataagaagccgacgggtatgattaagattcacgagatgaactccgagttgtctgtcctggcg

SEQ ID NO: 40 - DNA, 333 bp, Artificial Sequence
  CD86 variant 1044
gcccccctcaaaatccaagcgtacttcaacgaaactgcagacttaccgtgtcagtttgccaattcgcagaatctgaccctgagcgaactggtggttttctggcaggatcaggagaacctggttctgaacgaagtctatctgggcaaagagaaattcgacagcgtgtctagcaagtatatgggccgcaccagctttgatagcgacagctggaccctgcgtctgcacaatctgcaaatcaaagataagggtatctaccagtgcattatccaccataagaagccgacgggtatgattaagattcacgagatgagctccgagttgtctgtcctggcg

SEQ ID NO: 41 - DNA, 333 bp, Artificial Sequence
  CD86 variant 1045
gcccccctcaaaatccaagcgtacttcaacgaaactgcagacttaccgtgtcagtttgccaattcgcagaatctgaccctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcaaagataagggtctgtaccagtgcattatccaccataagaagccgacgggtctggtgaagattcacgagatgaactccgagttgtctgtcctggcg

SEQ ID NO: 42 - DNA, 333 bp, Artificial Sequence
  CD86 variant 1046
gcccccctcaaaatccaagcgtacttcaacgaaactgcagacttaccgtgtcagtttgccaattcgcagaatcaaagcctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcgaagataagggtatctaccagtgcattatccaccataagaagccgagcggtatggtgaagattcaccaaatggactccgagttgtctgtcctggcg

SEQ ID NO: 43 - DNA, 333 bp, Artificial Sequence
  CD86 variant 1047
gcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcaaagataagggtatctaccagtgcattatccaccataagaagccgacgggtctggtgaagattcacgagatgaactccgagttgtctgtcctggcg

SEQ ID NO: 44 - protein, 329 aa, Homo sapiens
  Human CD86
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 45 - protein, 223 aa, Mus <mouse, genus>
  Murine CTLA-4
MACLGLRRYKAQLQLPSRTWPFVALLTLLFIPVFSEAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSDFLLWILVAVSLGLFFYSFLVSAVSLSKMLKKRSPLTTGVYVKMPPTEPECEKQFQPYFIPIN

SEQ ID NO: 46 - protein, 218 aa, Mus <mouse, genus>
  Murine CD28
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQVTTMNMTPRRPGLTRKPYQPYAPARDFAAYRP

SEQ ID NO: 47 - protein, 11 aa, Artificial Sequence
  Linker
SGGGGSGGGGS

SEQ ID NO: 48 - protein, 13 aa, Artificial Sequence
  Linker
SGGGGSGGGGSAP

SEQ ID NO: 49 - protein, 5 aa, Artificial Sequence
  Linker
NFSQP

SEQ ID NO: 50 - protein, 5 aa, Artificial Sequence
  Linker
KRTVA

SEQ ID NO: 51 - protein, 277 aa, Homo sapiens
  Human OX40
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 52 - protein, 8 aa, Artificial Sequence
  1166 H CDR1
GFTFGGYY

SEQ ID NO: 53 - protein, 8 aa, Artificial Sequence
  1164 H CDR1
GFTFYGSS

SEQ ID NO: 54 - protein, 8 aa, Artificial Sequence
  1168 H CDR1
GFTFSGSS

SEQ ID NO: 55 - protein, 8 aa, Artificial Sequence
  1482 H CDR1
GFTFSSYA

SEQ ID NO: 56 - protein, 8 aa, Artificial Sequence
  1514 H CDR1
GFTFGYYY

SEQ ID NO: 57 - protein, 8 aa, Artificial Sequence
  1524 H CDR1
GFTFGSYY

SEQ ID NO: 58 - protein, 8 aa, Artificial Sequence
  1526 H CDR1
GFTFSGYS

SEQ ID NO: 59 - protein, 8 aa, Artificial Sequence
  1542 H CDR1
GFTFGSSS

SEQ ID NO: 60 - protein, 8 aa, Artificial Sequence
  1166 H CDR2
ISGSGGST

SEQ ID NO: 61 - protein, 8 aa, Artificial Sequence
  1170 H CDR2
IPGSGGST

SEQ ID NO: 62 - protein, 8 aa, Artificial Sequence
  1164 H CDR2
IYSSGGYT

SEQ ID NO: 63 - protein, 8 aa, Artificial Sequence
  1168 H CDR2
ISYYGGYT

SEQ ID NO: 64 - protein, 8 aa, Artificial Sequence
  1482 H CDR2
ISYYSGYT

SEQ ID NO: 65 - protein, 8 aa, Artificial Sequence
  1514 H CDR2
ISSYGSYT

SEQ ID NO: 66 - protein, 8 aa, Artificial Sequence
  1524 H CDR2
IGSYYGYT

SEQ ID NO: 67 - protein, 8 aa, Artificial Sequence
  1526 H CDR2
IGYSGYGT

SEQ ID NO: 68 - protein, 9 aa, Artificial Sequence
  1542 H CDR2
IGYYSYSTS

SEQ ID NO: 69 - protein, 10 aa, Artificial Sequence
  1166 H CDR3
ARYDYASMDY

SEQ ID NO: 70 - protein, 10 aa, Artificial Sequence
  1170 H CDR3
ARYDYYWMDY

SEQ ID NO: 71 - protein, 11 aa, Artificial Sequence
  1164 H CDR3
ARGVPHGYFDY

SEQ ID NO: 72 - protein, 11 aa, Artificial Sequence
  1168 H CDR3
ARYFPHYYFDY

SEQ ID NO: 73 - protein, 9 aa, Artificial Sequence
  1482 H CDR3
ARGYGYLDY

SEQ ID NO: 74 - protein, 11 aa, Artificial Sequence
  1490 H CDR3
ARYYPHHYIDY

SEQ ID NO: 75 - protein, 14 aa, Artificial Sequence
  1514 H CDR3
ARSGYSNWANSFDY

SEQ ID NO: 76 - protein, 17 aa, Artificial Sequence
  1520 H CDR3
ARYYYSHGYYVYGTLDY

SEQ ID NO: 77 - protein, 10 aa, Artificial Sequence
  1524 H CDR3
ARHDYGALDY

SEQ ID NO: 78 - protein, 15 aa, Artificial Sequence
  1526 H CDR3
ARYYFHDYAAYSLDY

SEQ ID NO: 79 - protein, 11 aa, Artificial Sequence
  1542 H CDR3
ARGYPHHYFDY

SEQ ID NO: 80 - protein, 6 aa, Artificial Sequence
  1167 L CDR1
QSISSY

SEQ ID NO: 81 - skipped

SEQ ID NO: 82 - protein, 10 aa, Artificial Sequence
  1167 L CDR3
QQYYWYGLST

SEQ ID NO: 83 - protein, 10 aa, Artificial Sequence
  1171 L CDR3
QQGHGSYPHT

SEQ ID NO: 84 - protein, 9 aa, Artificial Sequence
  1135 L CDR3
QQSYSTPYT

SEQ ID NO: 85 - protein, 8 aa, Artificial Sequence
  1483 L CDR3
QQYGSLLT

SEQ ID NO: 86 - protein, 9 aa, Artificial Sequence
  1515 L CDR3
QQGDYTLFT

SEQ ID NO: 87 - protein, 10 aa, Artificial Sequence
  1525 L CDR3
QQYGPSGLFT

SEQ ID NO: 88 - protein, 10 aa, Artificial Sequence
  1527 L CDR3
QQYGSDSLLT

SEQ ID NO: 89 - protein, 108 aa, Artificial Sequence
  1167, light chain VL
DIQMTQSPSSLSASVGDRVTITCRASQSISSYLNWYQQKPGKAPKLLIYAASSLQSGVPSRFSGSGSGTDFTLTISSLQPEDFATYYCQQYYWYGLSTFGQGTKLEIK

SEQ ID NO: 90 - DNA, 324 bp, Artificial Sequence
  1167, light chain VL
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtactactggtacggtctgtccacttttggccaggggaccaagctggagatcaaa

SEQ ID NO: 91 - protein, 117 aa, Artificial Sequence
  1166, heavy chain VH
EVQLLESGGGLVQPGGSLRLSCAASGFTFGGYYMSWVRQAPGKGLEWVSAISGSGGSTYYADSVKGRFTISRDNSKNTLYLQMNSLRAEDTAVYYCARYDYASMDYWGQGTLVTVSS

SEQ ID NO: 92 - DNA, 351 bp, Artificial Sequence
  1166, heavy chain VH
gaggtgcagctgttggagagcgggggaggcttggtacagcctggggggtccctgcgcctctcctgtgcagccagcggattcacctttggtggttactacatgtcttgggtccgccaggctccagggaaggggctggagtgggtctcagctattagtggtagtggtggtagcacatactatgcagactccgtgaagggccggttcaccatctcccgtgacaattccaagaacacgctgtatctgcaaatgaacagcctgcgtgccgaggacacggctgtatattattgtgcgcgctacgactacgcttctatggactattggggccagggaaccctggtcaccgtctcctca

SEQ ID NO: 93 - protein, 108 aa, Artificial Sequence
  1171, light chain VL
DIQMTQSPSSLSASVGDRVTITCRASQSISSYLNWYQQKPGKAPKLLIYAASSLQSGVPSRFSGSGSGTDFTLTISSLQPEDFATYYCQQGHGSYPHTFGQGTKLEIK

SEQ ID NO: 94 - DNA, 324 bp, Artificial Sequence
  1171, light chain VL
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagggtcatggttcttacccgcacacttttggccaggggaccaagctggagatcaaa

SEQ ID NO: 95 - protein, 117 aa, Artificial Sequence
  1170, heavy chain VH
EVQLLESGGGLVQPGGSLRLSCAASGFTFGGYYMSWVRQAPGKGLEWVSYIPGSGGSTYYADSVKGRFTISRDNSKNTLYLQMNSLRAEDTAVYYCARYDYYWMDYWGQGTLVTVSS

SEQ ID NO: 96 - DNA, 351 bp, Artificial Sequence
  1170, heavy chain VH
gaggtgcagctgttggagagcgggggaggcttggtacagcctggggggtccctgcgcctctcctgtgcagccagcggattcacctttggtggttactacatgtcttgggtccgccaggctccagggaaggggctggagtgggtctcatacattcctggttctggtggttctacatactatgcagactccgtgaagggccggttcaccatctcccgtgacaattccaagaacacgctgtatctgcaaatgaacagcctgcgtgccgaggacacggctgtatattattgtgcgcgctacgactactactggatggactattggggccagggaaccctggtcaccgtctcctca

SEQ ID NO: 97 - protein, 107 aa, Artificial Sequence
  1135, light chain VL
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIK

SEQ ID NO: 98 - DNA, 321 bp, Artificial Sequence
  1135, light chain VL
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagagttacagtaccccttatacttttggccaggggaccaagctggagatcaaa

SEQ ID NO: 99 - protein, 118 aa, Artificial Sequence
  1164, heavy chain VH
EVQLLESGGGLVQPGGSLRLSCAASGFTFYGSSMYWVRQAPGKGLEWVSGIYSSGGYTSYADSVKGRFTISRDNSKNTLYLQMNSLRAEDTAVYYCARGVPHGYFDYWGQGTLVTVSS

SEQ ID NO: 100 - DNA, 354 bp, Artificial Sequence
  1164, heavy chain VH
gaggtgcagctgttggagagcgggggaggcttggtacagcctggggggtccctgcgcctctcctgtgcagccagcggattcaccttttacggttcttctatgtactgggtccgccaggctccagggaaggggctggagtgggtctcaggtatttactcttctggtggttacacatcttatgcagactccgtgaagggccggttcaccatctcccgtgacaattccaagaacacgctgtatctgcaaatgaacagcctgcgtgccgaggacacggctgtatattattgtgcgcgcggtgttcctcatggttactttgactattggggccagggaaccctggtcaccgtctcctca

SEQ ID NO: 101 - protein, 118 aa, Artificial Sequence
  1168, heavy chain VH
EVQLLESGGGLVQPGGSLRLSCAASGFTFSGSSMSWVRQAPGKGLEWVSSISYYGGYTYYADSVKGRFTISRDNSKNTLYLQMNSLRAEDTAVYYCARYFPHYYFDYWGQGTLVTVSS

SEQ ID NO: 102 - DNA, 354 bp, Artificial Sequence
  1168, heavy chain VH
gaggtgcagctgttggagagcgggggaggcttggtacagcctggggggtccctgcgcctctcctgtgcagccagcggattcacctttagtggttcttctatgtcttgggtccgccaggctccagggaaggggctggagtgggtctcatctatttcttactacggtggttacacatactatgcagactccgtgaagggccggttcaccatctcccgtgacaattccaagaacacgctgtatctgcaaatgaacagcctgcgtgccgaggacacggctgtatattattgtgcgcgctacttcccgcattactactttgactattggggccagggaaccctggtcaccgtctcctca

SEQ ID NO: 103 - protein, 106 aa, Artificial Sequence
  1483, light chain VL
DIQMTQSPSSLSASVGDRVTITCRASQSISSYLNWYQQKPGKAPKLLIYAASSLQSGVPSRFSGSGSGTDFTLTISSLQPEDFATYYCQQYGSLLTFGQGTKLEIK

SEQ ID NO: 104 - DNA, 318 bp, Artificial Sequence
  1483, light chain VL
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtacggttctctgctcacttttggccaggggaccaagctggagatcaaa

SEQ ID NO: 105 - protein, 116 aa, Artificial Sequence
  1482, heavy chain VH
EVQLLESGGGLVQPGGSLRLSCAASGFTFSSYAMSWVRQAPGKGLEWVSYISYYSGYTYYADSVKGRFTISRDNSKNTLYLQMNSLRAEDTAVYYCARGYGYLDYWGQGTLVTVSS

SEQ ID NO: 106 - DNA, 348 bp, Artificial Sequence
  1482, heavy chain VH
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcatacatttcttactactctggttacacatactatgcagactccgtgaagggccggttcaccatctcccgtgacaattccaagaacacgctgtatctgcaaatgaacagcctgcgtgccgaggacacggctgtatattattgtgcgcgcggttacggttacttggactattggggccagggaaccctggtcaccgtctcctca

SEQ ID NO: 107 - protein, 118 aa, Artificial Sequence
  1490, heavy chain VH
EVQLLESGGGLVQPGGSLRLSCAASGFTFSSYAMSWVRQAPGKGLEWVSGISYYGGYTYYADSVKGRFTISRDNSKNTLYLQMNSLRAEDTAVYYCARYYPHHYIDYWGQGTLVTVSS

SEQ ID NO: 108 - DNA, 354 bp, Artificial Sequence
  1490, heavy chain VH
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcaggtatttcttactacggtggttacacatactatgcagactccgtgaagggccggttcaccatctcccgtgacaattccaagaacacgctgtatctgcaaatgaacagcctgcgtgccgaggacacggctgtatattattgtgcgcgctactacccgcatcattacattgactattggggccagggaaccctggtcaccgtctcctca

SEQ ID NO: 109 - protein, 107 aa, Artificial Sequence
  1515, light chain VL
DIQMTQSPSSLSASVGDRVTITCRASQSISSYLNWYQQKPGKAPKLLIYAASSLQSGVPSRFSGSGSGTDFTLTISSLQPEDFATYYCQQGDYTLFTFGQGTKLEIK

SEQ ID NO: 110 - DNA, 321 bp, Artificial Sequence
  1515, light chain VL
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagggtgattacactctgttcacttttggccaggggaccaagctggagatcaaa

SEQ ID NO: 111 - protein, 121 aa, Artificial Sequence
  1514, heavy chain VH
EVQLLESGGGLVQPGGSLRLSCAASGFTFGYYYMSWVRQAPGKGLEWVSGISSYGSYTYYADSVKGRFTISRDNSKNTLYLQMNSLRAEDTAVYYCARSGYSNWANSFDYWGQGTLVTVSS

SEQ ID NO: 112 - DNA, 363 bp, Artificial Sequence
  1514, heavy chain VH
gaggtgcagctgttggagagcgggggaggcttggtacagcctggggggtccctgcgcctctcctgtgcagccagcggattcacctttggttactactacatgtcttgggtccgccaggctccagggaaggggctggagtgggtctcaggtatttcttcttacggtagttacacatactatgcagactccgtgaagggccggttcaccatctcccgtgacaattccaagaacacgctgtatctgcaaatgaacagcctgcgtgccgaggacacggctgtatattattgtgcgcgctctggttactctaactgggctaactcttttgactattggggccagggaaccctggtcaccgtctcctca

SEQ ID NO: 113 - protein, 124 aa, Artificial Sequence
  1520, heavy chain VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARYYYSHGYYVYGTLDYWGQGTLVTVSS

SEQ ID NO: 114 - DNA, 372 bp, Artificial Sequence
  1520, heavy chain VH
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcagctattagtggtagtggtggtagcacatactatgcagactccgtgaagggccggttcaccatctcccgtgacaattccaagaacacgctgtatctgcaaatgaacagcctgcgtgccgaggacacggctgtatattattgtgcgcgctactactactctcatggttactacgtttacggtactttggactattggggccagggaaccctggtcaccgtctcctca

SEQ ID NO: 115 - protein, 108 aa, Artificial Sequence
  1525, light chain VL
DIQMTQSPSSLSASVGDRVTITCRASQSISSYLNWYQQKPGKAPKLLIYAASSLQSGVPSRFSGSGSGTDFTLTISSLQPEDFATYYCQQYGPSGLFTFGQGTKLEIK

SEQ ID NO: 116 - DNA, 324 bp, Artificial Sequence
  1525, light chain VL
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtacggtccgtctggtctgttcacttttggccaggggaccaagctggagatcaaa

SEQ ID NO: 117 - protein, 117 aa, Artificial Sequence
  1524, heavy chain VH
EVQLLESGGGLVQPGGSLRLSCAASGFTFGSYYMGWVRQAPGKGLEWVSSIGSYYGYTYYADSVKGRFTISRDNSKNTLYLQMNSLRAEDTAVYYCARHDYGALDYWGQGTLVTVSS

SEQ ID NO: 118 - DNA, 351 bp, Artificial Sequence
  1524, heavy chain VH
gaggtgcagctgttggagagcgggggaggcttggtacagcctggggggtccctgcgcctctcctgtgcagccagcggattcacctttggttcttactacatgggttgggtccgccaggctccagggaaggggctggagtgggtctcatctattggttcttactacggttacacatactatgcagactccgtgaagggccggttcaccatctcccgtgacaattccaagaacacgctgtatctgcaaatgaacagcctgcgtgccgaggacacggctgtatattattgtgcgcgccatgactacggtgctttggactattggggccagggaaccctggtcaccgtctcctca

SEQ ID NO: 119 - protein, 108 aa, Artificial Sequence
  1527, light chain VL
DIQMTQSPSSLSASVGDRVTITCRASQSISSYLNWYQQKPGKAPKLLIYAASSLQSGVPSRFSGSGSGTDFTLTISSLQPEDFATYYCQQYGSDSLLTFGQGTKLEIK

SEQ ID NO: 120 - DNA, 324 bp, Artificial Sequence
  1527, light chain VL
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtacggttctgattctctgctcacttttggccaggggaccaagctggagatcaaa

SEQ ID NO: 121 - protein, 122 aa, Artificial Sequence
  1526, heavy chain VH
EVQLLESGGGLVQPGGSLRLSCAASGFTFSGYSMYWVRQAPGKGLEWVSGIGYSGYGTYYADSVKGRFTISRDNSKNTLYLQMNSLRAEDTAVYYCARYYFHDYAAYSLDYWGQGTLVTVSS

SEQ ID NO: 122 - DNA, 366 bp, Artificial Sequence
  1526, heavy chain VH
gaggtgcagctgttggagagcgggggaggcttggtacagcctggggggtccctgcgcctctcctgtgcagccagcggattcaccttttctggttactctatgtactgggtccgccaggctccagggaaggggctggagtgggtctcaggtattggttactctggttacggtacatactatgcagactccgtgaagggccggttcaccatctcccgtgacaattccaagaacacgctgtatctgcaaatgaacagcctgcgtgccgaggacacggctgtatattattgtgcgcgctactacttccatgactacgctgcttactctttggactattggggccagggaaccctggtcaccgtctcctca

SEQ ID NO: 123 - protein, 118 aa, Artificial Sequence
  1542, heavy chain VH
EVQLLESGGGLVQPGGSLRLSCAASGFTFGSSSMYWVRQAPGKGLEWVSGIGYYSYSTSYADSVKGRFTISRDNSKNTLYLQMNSLRAEDTAVYYCARGYPHHYFDYWGQGTLVTVSS

SEQ ID NO: 124 - DNA, 354 bp, Artificial Sequence
  1542, heavy chain VH
gaggtgcagctgttggagagcgggggaggcttggtacagcctggggggtccctgcgcctctcctgtgcagccagcggattcacctttggttcttcttctatgtactgggtccgccaggctccagggaaggggctggagtgggtctcaggtattggttactactcttactctacatcttatgcagactccgtgaagggccggttcaccatctcccgtgacaattccaagaacacgctgtatctgcaaatgaacagcctgcgtgccgaggacacggctgtatattattgtgcgcgcggttacccgcatcattactttgactattggggccagggaaccctggtcaccgtctcctca

SEQ ID NO: 125 - protein, 337 aa, Artificial Sequence
  1261 = 1167 light chain VL, with constant kappa sequence, linker and CD86 mutant 1040
DIQMTQSPSSLSASVGDRVTITCRASQSISSYLNWYQQKPGKAPKLLIYAASSLQSGVPSRFSGSGSGTDFTLTISSLQPEDFATYYCQQYYWYGLSTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSAPLKIQAYFNETADLPCQFANSQNLSLSELVVFWQDQENLVLNEVYLGKERFDSVDSKYMGRTSFDSDSWTLRLHNLQIKDKGRYQCIIHHKKPTGMINIHQMNSELSVLA

SEQ ID NO: 126 - DNA, 1376 bp, Artificial Sequence
  1261 = 1267 light chain VL, with constant kappa sequence, linker and CD86 mutant 1040
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtactactggtacggtctgtccacttttggccaggggaccaagctggagatcaaacgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtagcggaggaggaggaagcggaggaggaggaagcgcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtaggtaccagtgcattatccaccataagaagccgacgggtatgattaatattcaccaaatgaactccgagttgtctgtcctggcg

SEQ ID NO: 127 - protein, 337 aa, Artificial Sequence
  1263= 1171 light chain VL, with constant kappa sequence, linker and CD86 mutant 1040
DIQMTQSPSSLSASVGDRVTITCRASQSISSYLNWYQQKPGKAPKLLIYAASSLQSGVPSRFSGSGSGTDFTLTISSLQPEDFATYYCQQGHGSYPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSAPLKIQAYFNETADLPCQFANSQNLSLSELVVFWQDQENLVLNEVYLGKERFDSVDSKYMGRTSFDSDSWTLRLHNLQIKDKGRYQCIIHHKKPTGMINIHQMNSELSVLA

SEQ ID NO: 128 - DNA, 1376 bp, Artificial Sequence
  1263= 1171 light chain VL, with constant kappa sequence, linker and CD86 mutant 1040
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagggtcatggttcttacccgcacacttttggccaggggaccaagctggagatcaaacgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtagcggaggaggaggaagcggaggaggaggaagcgcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtaggtaccagtgcattatccaccataagaagccgacgggtatgattaatattcaccaaatgaactccgagttgtctgtcctggcg

SEQ ID NO: 129 - protein, 336 aa, Artificial Sequence
  1141= 1135 light chain VL, with constant kappa sequence, linker and CD86 mutant 1040
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSAPLKIQAYFNETADLPCQFANSQNLSLSELVVFWQDQENLVLNEVYLGKERFDSVDSKYMGRTSFDSDSWTLRLHNLQIKDKGRYQCIIHHKKPTGMINIHQMNSELSVLA

SEQ ID NO: 130 - DNA, 1373 bp, Artificial Sequence
  1141= 1135 light chain VL, with constant kappa sequence, linker and CD86 mutant 1040
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagagttacagtaccccttatacttttggccaggggaccaagctggagatcaaacgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtagcggaggaggaggaagcggaggaggaggaagcgcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtaggtaccagtgcattatccaccataagaagccgacgggtatgattaatattcaccaaatgaactccgagttgtctgtcctggcg

SEQ ID NO: 131 - protein, 336 aa, Artificial Sequence
  1581= 1515 light chain VL, with constant kappa sequence, linker and CD86 mutant 1040
DIQMTQSPSSLSASVGDRVTITCRASQSISSYLNWYQQKPGKAPKLLIYAASSLQSGVPSRFSGSGSGTDFTLTISSLQPEDFATYYCQQGDYTLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSAPLKIQAYFNETADLPCQFANSQNLSLSELVVFWQDQENLVLNEVYLGKERFDSVDSKYMGRTSFDSDSWTLRLHNLQIKDKGRYQCIIHHKKPTGMINIHQMNSELSVLA

SEQ ID NO: 132 - DNA, 1373 bp, Artificial Sequence
  1581= 1515 light chain VL, with constant kappa sequence, linker and CD86 mutant 1040
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagggtgattacactctgttcacttttggccaggggaccaagctggagatcaaacgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtagcggaggaggaggaagcggaggaggaggaagcgcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtaggtaccagtgcattatccaccataagaagccgacgggtatgattaatattcaccaaatgaactccgagttgtctgtcctggcg

SEQ ID NO: 133 - protein, 337 aa, Artificial Sequence
  1585= 1527 light chain VL, with constant kappa sequence, linker and CD86 mutant 1040
DIQMTQSPSSLSASVGDRVTITCRASQSISSYLNWYQQKPGKAPKLLIYAASSLQSGVPSRFSGSGSGTDFTLTISSLQPEDFATYYCQQYGSDSLLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSAPLKIQAYFNETADLPCQFANSQNLSLSELVVFWQDQENLVLNEVYLGKERFDSVDSKYMGRTSFDSDSWTLRLHNLQIKDKGRYQCIIHHKKPTGMINIHQMNSELSVLA

SEQ ID NO: 134 - DNA, 1376 bp, Artificial Sequence
  1585= 1527 light chain VL, with constant kappa sequence, linker and CD86 mutant 1040
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtacggttctgattctctgctcacttttggccaggggaccaagctggagatcaaacgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtagcggaggaggaggaagcggaggaggaggaagcgcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtaggtaccagtgcattatccaccataagaagccgacgggtatgattaatattcaccaaatgaactccgagttgtctgtcctggcg

SEQ ID NO: 135 - protein, 330 aa, Homo sapiens [sequence omitted]

SEQ ID NO: 136 - protein, 107 aa, Homo sapiens [sequence omitted]

SEQ ID NO: 137 - protein, 327 aa, Artificial Sequence
  Modified human heavy chain IgG4 constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRYTQKSLSLSLGK

SEQ ID NO: 138 - protein, 327 aa, Homo sapiens
  Wild type human heavy chain IgG4 constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 139 - protein, 327 aa, Artificial Sequence
  Modified human heavy chain IgG4 constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 140 - DNA, 981 bp, Artificial Sequence
  cDNA sequence encoding the IgG4 constant region of SEQ ID NO: 137
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccttgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccgctacacacagaagagcctctccctgtctctgggtaaa

SEQ ID NO: 141 - DNA, 2028 bp, Artificial Sequence
  Genomic DNA sequence encoding the IgG4 constant region of SEQ ID NO: 137
agctttctggggcaggccgggcctgactttggctgggggcagggagggggctaaggtgacgcaggtggcgccagccaggtgcacacccaatgcccatgagcccagacactggaccctgcatggaccatcgcggatagacaagaaccgaggggcctctgcgccctgggcccagctctgtcccacaccgcggtcacatggcaccacctctcttgcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgctcagggagagggtcttctggatttttccaccaggctcccggcaccacaggctggatgcccctaccccaggccctgcgcatacagggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccaccttgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacacggacagaggccagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccgctacacacagaagagcctctccctgtctctgggtaaatgagtgccagggccggcaagcccccgctccccgggctctcggggtcgcgcgaggatgcttggcacgtaccccgtctacatacttcccaggcacccagcatggaaataaagcacccaccactgccctgggcccctgtgagactgtgatggttctttccacgggtcaggccgagtctgaggcctgagtgacatgagggaggcagagcgggtcccactgtccccacactgg

SEQ ID NO: 142 - DNA, 981 bp, Homo sapiens
  cDNA sequence encoding the IgG4 constant region of SEQ ID NO: 138
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 143 - DNA, 2028 bp, Homo sapiens
  Genomic DNA sequence encoding the IgG4 constant region of SEQ ID NO: 138
agctttctggggcaggccgggcctgactttggctgggggcagggagggggctaaggtgacgcaggtggcgccagccaggtgcacacccaatgcccatgagcccagacactggaccctgcatggaccatcgcggatagacaagaaccgaggggcctctgcgccctgggcccagctctgtcccacaccgcggtcacatggcaccacctctcttgcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgctcagggagagggtcttctggatttttccaccaggctcccggcaccacaggctggatgcccctaccccaggccctgcgcatacagggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccatcatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacacggacagaggccagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccgggctctcggggtcgcgcgaggatgcttggcacgtaccccgtctacatacttcccaggcacccagcatggaaataaagcacccaccactgccctgggcccctgtgagactgtgatggttctttccacgggtcaggccgagtctgaggcctgagtgacatgagggaggcagagcgggtcccactgtccccacactgg

SEQ ID NO: 144 - protein, 15 aa, Artificial Sequence
  Linker
GGGGSGGGGSGGGGS

SEQ ID NO: 145 - DNA, 990 bp, Homo sapiens
  cDNA sequence encoding the IgG1 constant region of SEQ ID NO: 135
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 146 - DNA, 1596 bp, Homo sapiens
  Genomic DNA sequence encoding the IgG1 constant region of SEQ ID NO: 135
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 147 - DNA, 321 bp, Homo sapiens
  Light chain kappa region of SEQ ID NO: 136
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 148 - DNA, 981 bp, Artificial Sequence
  cDNA sequence encoding the IgG4 region of SEQ ID NO: 139
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccttgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 149 - DNA, 2028 bp, Artificial Sequence
  Genomic DNA sequence encoding the IgG4 region of SEQ ID NO: 139.
agctttctggggcaggccgggcctgactttggctgggggcagggagggggctaaggtgacgcaggtggcgccagccaggtgcacacccaatgcccatgagcccagacactggaccctgcatggaccatcgcggatagacaagaaccgaggggcctctgcgccctgggcccagctctgtcccacaccgcggtcacatggcaccacctctcttgcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgctcagggagagggtcttctggatttttccaccaggctcccggcaccacaggctggatgcccctaccccaggccctgcgcatacagggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccaccttgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacacggacagaggccagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccgggctctcggggtcgcgcgaggatgcttggcacgtaccccgtctacatacttcccaggcacccagcatggaaataaagcacccaccactgccctgggcccctgtgagactgtgatggttctttccacgggtcaggccgagtctgaggcctgagtgacatgagggaggcagagcgggtcccactgtccccacactgg

SEQ ID NO: 150 - protein, 4 aa, Artificial Sequence
  Linker (SG)m where m=2
SGSG

SEQ ID NO: 151 - protein, 6 aa, Artificial Sequence
  Linker (SG)m where m=3
SGSGSG

SEQ ID NO: 152 - protein, 8 aa, Artificial Sequence
  Linker (SG)m where m=4
SGSGSGSG

SEQ ID NO: 153 - protein, 10 aa, Artificial Sequence
  Linker (SG)m where m=5
SGSGSGSGSG

SEQ ID NO: 154 - protein, 12 aa, Artificial Sequence
  Linker (SG)m where m=6
SGSGSGSGSGSG

SEQ ID NO: 155 - protein, 14 aa, Artificial Sequence
  Linker (SG)m where m=7
SGSGSGSGSGSGSG

SEQ ID NO: 156 - protein, 8 aa, Artificial Sequence
  Heavy chain CDR1 consensus sequence
  VARIANT 5: Gly or Tyr or Ser
  VARIANT 6: Gly or Tyr or Ser
  VARIANT 7: Tyr or Ser
  VARIANT 8: Tyr or Ser or Ala
GFTFXXXX

SEQ ID NO: 157 - protein, 8 aa, Artificial Sequence
  Heavy chain CDR2 consensus sequence
  VARIANT 2: Gly or Tyr or Ser or Thr
  VARIANT 3: Gly or Ser or Tyr
  VARIANT 4: Ser or Tyr
  VARIANT 5: Gly or Ser or Tyr
  VARIANT 6: Gly or Ser or Tyr
  VARIANT 7: Gly or Ser or Tyr
IXXXXXXT

SEQ ID NO: 158 - protein, 17 aa, Artificial Sequence
  Heavy chain CDR3 consensus sequence
  VARIANT 3: Gly or Tyr or Ser or His
  VARIANT 4: Gly or Tyr or Phe or Val or Asp
  VARIANT 5: Gly or Tyr or Pro or Phe
  VARIANT 6: No amino acid or His or Ser
  VARIANT 7: No amino acid or Asn or Asp or His
  VARIANT 8: No amino acid or Tyr or Gly
  VARIANT 9: No amino acid or Tyr
  VARIANT 10: No amino acid or Tyr
  VARIANT 11: No amino acid or Trp or Ala or Val
  VARIANT 12: No amino acid or Ala or Tyr
  VARIANT 13: No amino acid or Asp or Ala or Tyr or Gly or His or Asn
  VARIANT 14: Tyr or Ser or Trp or Ala or Thr
  VARIANT 15: Leu or Met or Ile or Phe
ARXXXXXXXXXXXXXDY

SEQ ID NO: 159 - protein, 10 aa, Artificial Sequence
  Light chain CDR3 consensus sequence
  VARIANT 3: Ser or Tyr or Gly
  VARIANT 4: No amino acid or Tyr or His or Gly
  VARIANT 5: No amino acid or Ser or Tyr or Gly or Asp or Trp
  VARIANT 6: Ser or Tyr or Gly or Asp
  VARIANT 7: Ser or Tyr or Gly or Thr
  VARIANT 8: Pro or Leu
  VARIANT 9: Tyr or Ser or His or Leu or Phe
QQXXXXXXXT

SEQ ID NO: 160 - protein, 10 aa, Artificial Sequence
  VH CDR3 first sub-family consensus sequence
  VARIANT 3: Tyr or His
  VARIANT 6: Ala or Tyr or Gly
  VARIANT 7: Ser or Trp or Ala
  VARIANT 8: Met or Leu
ARXDYXXXDY

SEQ ID NO: 161 - protein, 11 aa, Artificial Sequence
  VH CDR3 second sub-family consensus sequence
  VARIANT 3: Gly or Tyr
  VARIANT 4: Val or Phe or Tyr
  VARIANT 7: Gly or Tyr or His
  VARIANT 9: Phe or Ile
ARXXPHXYXDY